(19) United States
(12) Patent Application Publication
(10) Pub. No.: US 2014/0106418 A1
(43) Pub. Date: Apr. 17, 2014

(54) Enhanced Fermentation From Pretreatment Products

(71) Applicants: Sarad Parekh, Pittsford, NY (US); Carl P. Felice, Churchville, NY (US); Benjamin Stauber, Rochester, NY (US)

(72) Inventors: Sarad Parekh, Pittsford, NY (US); Carl P. Felice, Churchville, NY (US); Benjamin Stauber, Rochester, NY (US)

(73) Assignee: Sweetwater Energy, Inc., Rochester, NY (US)

(21) Appl. No.: 13/842,941

(22) Filed: Mar. 15, 2013

Related U.S. Application Data
(60) Provisional application No. 61/648,567, filed on May 17, 2012.
(60) Provisional application No. 61/615,588, filed on Mar. 26, 2012.
(60) Provisional application No. 61/662,339, filed on Jun. 20, 2012.

(51) Int. Cl. C12P 7/64; C12P 7/20

(52) U.S. Cl. CPC C12P 7/6436; C12P 7/20; C12P 7/6409
USPC 435/134; 435/159

(57) ABSTRACT

Provided are methods and compositions for higher yields through the addition of pretreatment/hydrolysis extracts in the fermentation process. These fractions increase the rate of growth for biocatalysts and result in improved composition of fermentation end products.

### CROSS-REFERENCE

This application claims the benefit of U.S. Provisional Application Nos. 61/615,588, filed Mar. 26, 2012, and 61/648,567, filed May 17, 2012, each of which application is incorporated herein by reference in its entirety.

### BACKGROUND

Various options for generating effective and sustainable biofuels and other biochemicals have been suggested. For example, microbial biocatalyst fermentation from biomass containing polymers such as cellulose, lignocellulose, pectin, starch and/or xylose can provide much needed solutions for the world energy problem. Species of yeast, fungi and bacteria have been reported to be able to convert carbonaceous biomass to monomeric sugars and subsequently to alcohols, fuels and other chemical products. However, many of these microorganisms grow slowly on extracted sugar solutions and/or produce desired chemicals only at low concentrations. Even yeasts, which grow fairly rapidly when consuming sugar, have a lag phase that can result in lower efficiencies and initial contamination by undesirable organisms. Such product production issues, in addition to affecting the chemical titers, can also affect overall efficiency and productivity.

Media components are an important part of fermentation. Biomass provides carbon sources and some essential growth factors for fungal and bacterial biocatalysts, but not all. In addition to affecting the total cost of the products produced from biomass, growth media components control the effectiveness of biocatalysts and the compositions of products. Maximizing growth factors in the pretreatment and hydrolysis of biomass can boost the efficiency of biocatalysts while reducing the costs of additional media chemicals. The effects of such treatments can also be exploited to increase the yields of particular fermentation products.

### SUMMARY

Disclosed herein are methods of producing one or more fermentation end-products comprising: a. contacting a saccharide solution comprising C5 monosaccharides and/or C6 monosaccharides and particulate solids with a cell culture; and b. allowing sufficient time for cells in the cell culture to produce one or more fermentation end-products comprising one or more polyols, fatty acids, triacylglycerols, or a combination thereof; wherein a greater yield of the one or more polyols, fatty acids, or triacylglycerols is produced in comparison to fermentation of a saccharide solution comprising a lower level of the particulate solids. In some embodiments, the saccharide solution further comprises one or more osmotic agents. Some embodiments further comprise adding one or more exogenous osmotic agents to the saccharide solution. Some embodiments further comprise adding exogenous particulate solids to the saccharide solution.

Also disclosed herein are methods of producing one or more fermentation end-products comprising: a. adding exogenous particulate solids to a saccharide solution comprising C5 monosaccharides and/or C6 monosaccharides; b. contacting the saccharide solution with a cell culture; and c. allowing sufficient time for cells in the cell culture to produce one or more fermentation end-products comprising one or more polyols, fatty acids, triacylglycerols, or a combination thereof; wherein a greater yield of the one or more polyols, fatty acids, or triacylglycerols is produced in comparison to fermentation of a saccharide solution without the exogenous particulate solids. In some embodiments, the saccharide solution further comprises particulate solids. In some embodiments, the saccharide solution further comprises one or more osmotic agents. Some embodiments further comprise adding one or more exogenous osmotic agents to the saccharide solution.

Also disclosed herein are methods of producing one or more fermentation end-products comprising: a. adding one or more exogenous osmotic agents to a saccharide solution comprising C5 monosaccharides and/or C6 monosaccharides; b. contacting the saccharide solution with a cell culture; and c. allowing sufficient time for cells in the cell culture to produce one or more fermentation end-products comprising one or more polyols, fatty acids, triacylglycerols, or a combination thereof; wherein a greater yield of the one or more polyols, fatty acids, or triacylglycerols is produced in comparison to fermentation of a saccharide solution without the one or more exogenous osmotic agents. In some embodiments, the saccharide solution further comprises one or more osmotic agents. In some embodiments, the saccharide solution further comprises particulate solids. Some embodiments further comprise adding exogenous particulate solids to the saccharide solution.

In some embodiments, the saccharide solution was produced by pretreating and hydrolyzing a biomass composition comprising cellulosic, hemicellulosic, and/or lignocellulosic material. In some embodiments, the biomass comprising cellulosic, hemicellulosic, and/or lignocellulosic material is corn, corn syrup, corn stover, corn cobs, molasses, silage, grass, straw, grain hulls, bagasse, distiller's grains, distiller's dried solubles, distiller's dried grains, condensed distiller's solubles, distiller's wet grains, distiller's dried grains with solubles, wood, bark, sawdust, paper, poplars, willows, switchgrass, alfalfa, prairie bluestem, algae, fruit peels, pits, sorghum, sweet sorghum, sugar cane, switch grass, rice, rice straw, rice hulls, wheat, wheat straw, barley, barley straw, bamboo, seeds, seed hulls, oats, oat hulls, food waste, municipal sewage waste, or a combination thereof. In some embodiments, pretreating and hydrolyzing comprises mechanical size reduction, treatment with one or more acids, treatment with one or more bases, treatment with one or more enzymes, thermal treatment, stream explosion, acid-catalyzed steam explosion, ammonia fiber explosion, or a combination thereof.

In some embodiments, the one or more fermentation end-products comprise one or more polyols that are glycol, glycerol, erythritol, threitol, arabitol, xylitol, ribitol, mannitol, sorbitol, dulcitol, fucitol, iditol, inositol, volemitol, isomalt, maltitol, lactitol, polyglycitol. In some embodiments, the one or more fermentation end-products comprise glycerol. In some embodiments, the one or more fermentation end-products comprise one or more fatty acids that are that are butyric acid, hexanoic acid, octanoic acid, decanoic acid, lauric acid, tridecanoic acid, myristic acid, pentadecanoic acid, palmitic acid, heptadecanoic acid, stearic acid, arachidic acid, heneicosanoic acid, behenic acid, tricosanoic acid, lignoceric acid, (cis-9) myristoleic acid, (cis-10) pentadecinoic acid, (cis-9) palmitoleic acid, (cis-10) heptadecenoate acid, (cis-9) oleic acid, (cis-11) eicosenoic acid, (cis-13) erucic acid, (cis-15) nervonic acid, (cis-9, 12) lonoleic acid, (cis-6, 9, 12) y-linolenic acid, (cis-9, 12,15) linolenic acid, (cis-11, 14) eicosadienoic acid, (cis-8, 11,14) eicosatrienoic acid, (cis-11, 14, 17) eicosatrienoic acid, (cis-5, 8, 11, 14) arachidonic acid, (cis-5, 8, 11, 14, 17) eicosapentanoic acid, (cis-13, 16) docosadienoic acid, (cis-4, 7, 10, 13, 16, 19) docosahexaenoic acid, (trans-9) methyl elaidate acid, (trans-9, 12) methyl linoelaidate acid, or a combination thereof. In some embodiments, the one or more fermentation end-products comprise one or more triacylglycerols.

In some embodiments, the particulate solids comprise lignin, cellulose, hemicellulose, or a combination thereof. In some embodiments, the particulate solids are residual solids from pretreating and hydrolyzing a biomass. In some embodiments, the particulate solids have a particle size of from about 1 μm to about 5 mm. In some embodiments, the particulate solids have a particle size of from about 100 μm to about 2.5 mm. In some embodiments, the particulate solids have a particle size of from about 250 μm to about 1 mm. In some embodiments, the particulate solids have an average particle size of less than about 5 mm. In some embodiments, the particulate solids have an average particle size of less than about 1 mm. In some embodiments, the particulate solids have an average particle size of less than about 500 μm. In some embodiments, the particulate solids have an average particle size of less than about 250 μm. In some embodiments, the amount of particulate solids in the saccharide solution is from about 0.001% to about 30% w/v. In some embodiments, the amount of particulate solids in the saccharide solution is from about 0.01% to about 20% w/v. In some embodiments, the amount of particulate solids in the saccharide solution is from about 0.1% to about 10% w/v. In some embodiments, a growth rate of cells in the cell culture is faster in the saccharide solution in comparison to the saccharide solution comprising the lower level of the particulate solids.

In some embodiments, the exogenous particulate solids comprise lignin, cellulose, hemicellulose, or a combination thereof. In some embodiments, the exogenous particulate solids are residual solids that were collected following pretreatment and hydrolysis of a biomass. In some embodiments, the exogenous particulate solids have a particle size of from about 1 μm to about 5 mm. In some embodiments, the exogenous particulate solids have a particle size of from about 100 μm to about 2.5 mm. In some embodiments, the exogenous particulate solids have a particle size of from about 250 μm to about 1 mm. In some embodiments, the exogenous particulate solids have an average particle size of less than about 5 mm. In some embodiments, the exogenous particulate solids have an average particle size of less than about 1 mm. In some embodiments, the exogenous particulate solids have an average particle size of less than about 500 μm. In some embodiments, the exogenous particulate solids have an average particle size of less than about 250 μm. In some embodiments, the exogenous particulate solids are added to the saccharide solution to from about 0.001% to about 30% w/v. In some embodiments, the exogenous particulate solids are added to the saccharide solution to from about 0.01% to about 20% w/v. In some embodiments, the exogenous particulate solids are added to the saccharide solution to from about 0.1% to about 10% w/v. In some embodiments, a growth rate of cells in the cell culture is faster in saccharide solutions with the exogenous particulate solids than in saccharide solutions without the exogenous particulate solids.

In some embodiments, the one or more osmotic agents comprise one or more salts, acid solubilized lignin, one or more fatty acids, one or more metal ions, one or more trace elements, one or more acids, one or more bases, ash, one or more organic acids, one or more alcohols, or a combination thereof. In some embodiments, the one or more osmotic agents comprise one or more metal ions that are aluminum ions, antimony ions, arsenic ions, barium ions, cadmium ions, calcium ions, chromium ions, cobalt ions, copper ions, iron ions, lead ions, magnesium ions, manganese ions, nickel ions, phosphorus ions, potassium ions, selenium ions, silver ions, sodium ions, tin ions, vanadium ions, zinc ions, or a combination thereof. In some embodiments, the one or more osmotic agents comprise one or more salts that were formed by neutralization of an acid or a base following pretreatment of a biomass.

In some embodiments, the one or more exogenous osmotic agents comprise one or more salts, acid solubilized lignin, one or more fatty acids, one or more metal ions, one or more trace elements, one or more acids, one or more bases, ash, one or more organic acids, one or more alcohols, or a combination thereof. In some embodiments, the one or more exogenous osmotic agents comprise one or more salts, one or more minerals, one or more metal ions, or a combination thereof. In some embodiments, the one or more exogenous osmotic agents comprise one or more metal ions that are aluminum ions, antimony ions, arsenic ions, barium ions, cadmium ions, calcium ions, chromium ions, cobalt ions, copper ions, iron ions, lead ions, magnesium ions, manganese ions, nickel ions, phosphorus ions, potassium ions, selenium ions, silver ions, sodium ions, tin ions, vanadium ions, zinc ions, or a combination thereof. In some embodiments, adding the one or more exogenous osmotic agents increases the osmolarity of the saccharide solution by from about 0.01% to about 50%. In some embodiments, adding the one or more exogenous osmotic agents increases the osmolarity of the saccharide solution by from about 0.01% to about 10%. In some embodiments, adding the one or more exogenous osmotic agents increases the osmolarity of the saccharide solution by at least about 0.01%, 0.1%, 0.5%, 1%, 1.5%, 2%, 3%, 4%, 5%, 6%, 7%, 8%, 9%, 10%, or more.

In some embodiments, the cell culture comprises plant cells, bacterial cells, yeast cells, algal cells, or a combination thereof. In some embodiments, the cell culture comprises genetically modified cells. In some embodiments, the cell culture comprises a Rhodococcus strain, a Clostridium strain, a Trichoderma strain, a Saccharomyces strain, a Zymomonas strain, or a combination thereof. In some embodiments, the cell culture comprises a gram⁺ bacterium. In some embodiments, the cell culture comprises a gram⁻ bacterium. In some embodiments, the cell culture comprises a Rhodococcus opacus strain. In some embodiments, the cell culture comprises a genetically modified Rhodococcus opacus strain.

In some embodiments, the C5 saccharides and/or C6 saccharides are at a concentration of from about 0.1% w/v to about 50% w/v in the saccharide solution. In some embodiments, the C5 saccharides and/or C6 saccharides are at a concentration of from about 0.1% w/v to about 25% w/v in the saccharide solution. In some embodiments, the C5 saccharides and/or C6 saccharides are at a concentration of from about 0.1% w/v to about 5% w/v in the saccharide solution.

In some embodiments, the greater yield is at least about 1% higher. In some embodiments, the greater yield is at least about 10% higher. In some embodiments, the greater yield is at least about 50% higher. In some embodiments, the greater yield is at least about 75% higher. In some embodiments, the greater yield is at least about two fold higher.

In some embodiments, a yield of one or more other fermentation end-products is lower in comparison to fermentation of a saccharide solution comprising a lower level of the particulate solids. In some embodiments, the one or more other fermentation end-products comprise one or more alcohols. In some embodiments, the one or more other fermentation end-products comprise ethanol. In some embodiments, a yield of ethanol is lower in comparison to fermentation of a saccharide solution comprising a lower level of the particulate solids. In some embodiments, a yield of ethanol is insubstantially affected in comparison to fermentation of a saccharide solution comprising a lower level of the particulate solids. In some embodiments, a yield of ethanol is not affected in comparison to fermentation of a saccharide solution comprising a lower level of the particulate solids.

Also provided herein are the triacylglycerols, polyols, and/or glycerol produced by these methods.

In another aspect, provided herein are systems for producing an increased yield of polyols, fatty acids, and/or triacylglycerols, the system comprising: a. a fermentation vessel; b. a saccharide solution comprising C5 monosaccharides and/or C6 monosaccharides and particulate solids; and c. a cell culture comprising cells that produce one or more polyols, fatty acids and/or triacylglycerols from the C5 monosaccharides and/or the C6 monosaccharides in a greater yield than from an equivalent amount of the C5 monosaccharides and/or C6 monosaccharides with a lower level of the particulate solids.

Also disclosed herein are systems for producing an increased yield of polyols, fatty acids, and/or triacylglycerols, the system comprising: a. a fermentation vessel; b. a saccharide solution comprising C5 monosaccharides and/or C6 monosaccharides and one or more osmotic agents; and c. a cell culture comprising cells that produce one or more polyols, fatty acids and/or triacylglycerols from the C5 monosaccharides and/or the C6 monosaccharides in a greater yield than from an equivalent amount of the C5 monosaccharides and/or C6 monosaccharides with a lower level of the one or more osmotic agents.

Some embodiments further comprise a fatty acid extractor. Some embodiments further comprise a cell separator.

In some embodiments, the saccharide solution further comprises particulate solids. In some embodiments, the particulate solids comprise lignin, cellulose, hemicellulose, or a combination thereof. In some embodiments, the particulate solids are residual solids from pretreating and hydrolyzing a biomass. In some embodiments, the particulate solids have a particle size of from about 1 μm to about 5 mm, 100 μm to about 2.5 mm, or 250 μm to about 1 mm. In some embodiments, the particulate solids have an average particle size of less than about 5 mm, 1 mm, 500 μm, or 250 μm. In some embodiments, the amount of particulate solids in the saccharide solution is from about 0.001% to about 30% w/v, about 0.01% to about 20% w/v, or 0.1% to about 10% w/v.

In some embodiments, the saccharide solution further comprises one or more osmotic agents. In some embodiments, the particulate solids comprise exogenous particulate solids. In some embodiments, the one or more osmotic agents comprise one or more salts, acid solubilized lignin, one or more fatty acids, one or more metal ions, one or more trace elements, one or more acids, one or more bases, ash, one or more organic acids, one or more alcohols, or a combination thereof. In some embodiments, the one or more osmotic agents comprise one or more salts that were formed by neutralization of an acid or a base following pretreatment of a biomass. In some embodiments, the one or more osmotic agents comprise one or more metal ions that are aluminum ions, antimony ions, arsenic ions, barium ions, cadmium ions, calcium ions, chromium ions, cobalt ions, copper ions, iron ions, lead ions, magnesium ions, manganese ions, nickel ions, phosphorus ions, potassium ions, selenium ions, silver ions, sodium ions, tin ions, vanadium ions, zinc ions, or a combination thereof. In some embodiments, the one or more osmotic agents comprise one or more salts that were formed by neutralization of an acid or a base following pretreatment of a biomass. In some embodiments, the one or more osmotic agents increases the osmolarity of the saccharide solution by from about 0.01% to about 50%, or about 0.01% to about 10%. In some embodiments, the one or more osmotic agents increases the osmolarity of the saccharide solution by at least about 0.01%, 0.1%, 0.5%, 1%, 1.5%, 2%, 3%, 4%, 5%, 6%, 7%, 8%, 9%, 10%, or more. In some embodiments, the one or more osmotic agents comprise exogenous osmotic agents.

In some embodiments, the cell culture comprises plant cells, bacterial cells, yeast cells, algal cells, or a combination thereof. In some embodiments, the C5 saccharides and/or C6 saccharides are at a concentration of from about 0.1% w/v to about 50%, about 0.1% w/v to about 25%, or about 0.1% w/v to about 5% w/v in the saccharide solution.

In another aspect, provided herein are fermentation substrates comprising: a. C5 monosaccharides; b. C6 monosaccharides; and c. one or more metal ions comprising aluminum ions, antimony ions, arsenic ions, barium ions, cadmium ions, calcium ions, chromium ions, cobalt ions, copper ions, iron ions, lead ions, magnesium ions, manganese ions, nickel ions, phosphorus ions, potassium ions, selenium ions, silver ions, sodium ions, tin ions, vanadium ions, zinc ions, or a combination thereof.

In some embodiments, the C5 monosaccharides and the C6 monosaccharides were produced by pretreating and hydrolyzing a biomass composition comprising cellulose, hemicellulose, and/or lignocellulose. In some embodiments, the C5 saccharides and/or C6 saccharides are at a concentration of from about 0.1% w/v to about 50%, about 0.1% w/v to about 25%, or about 0.1% w/v to about 5% w/v. In some embodiments, a ratio of the C5 monosaccharides to C6 monosaccharides is about 1:99, 2:98, 3:97, 4:96, 5:95, 7.5:92.5, 10:90, 20:80, 30:70, 40:60, 50:50, 60:40, 70:30, 80:20, 90:10, 95:5, or 99:1.

Some embodiments further comprise particulate solids comprising cellulose, hemicellulose, and/or lignin. In some embodiments, the particulate solids are in an amount of from about 0.001% to about 30% w/v, about 0.01% to about 20% w/v, or 0.1% to about 10% w/v. In some embodiments, the particulate solids are residual solids from pretreating and hydrolyzing a biomass. In some embodiments, the particulate solids have a particle size of from about 1 μm to about 5 mm, 100 μm to about 2.5 mm, or 250 μm to about 1 mm. In some embodiments, the particulate solids have an average particle size of less than about 5 mm, 1 mm, 500 μm, or 250 μm.

Some embodiments comprise at least 2, 3, 4, 5, 6, 7, 8, 9, 10, 11, 12, 13, 14, 15, 16, 17, 18, 19, 20, 21, or 22 of the metal ions. In some embodiments, the one or more metal ions comprise from about 0.01 PPM to about 2.5 PPM of at least one of antimony ions or arsenic ions. In some embodiments, the one or more metal ions comprise from about 0.05 PPM to about 25 PPM of at least one of cadmium ions, cobalt ions, lead ions, selenium ions, silver ions, tin ions, or vanadium ions. In some embodiments, the one or more metal ions comprise from about 0.1 PPM to about 500 PPM of at least one of aluminum ions, iron ions, magnesium ions, or phosphorus. In some embodiments, the one or more metal ions comprise from about 10 PPM to about 5000 PPM of at least one of potassium ions, calcium ions, or sodium ions.

In some embodiments, the one or more metal ions comprise aluminum ions in an amount of from about 1 PPM to about 200 PPM. In some embodiments, the one or more metal ions comprise antimony ions in an amount of from about 0.01 PPM to about 1 PPM. In some embodiments, the one or more metal ions comprise arsenic ions in an amount of from about 0.1 PPM to about 1 PPM. In some embodiments, the one or more metal ions comprise barium ions in an amount of from about 0.01 PPM to about 10 PPM. In some embodiments, the one or more metal ions comprise cadmium ions in an amount of from about 0.001 PPM to about 0.5 PPM. In some embodiments, the one or more metal ions comprise calcium ions in an amount of from about 10 PPM to about 1500 PPM. In some embodiments, the one or more metal ions comprise chromium ions in an amount of from about 0.01 PPM to about 25 PPM. In some embodiments, the one or more metal ions comprise cobalt ions in an amount of from about 0.01 PPM to about 1 PPM. In some embodiments, the one or more metal ions comprise copper ions in an amount of from about 0.1 PPM to about 25 PPM. In some embodiments, the one or more metal ions comprise iron ions in an amount of from about 0.1 PPM to about 500 PPM. In some embodiments, the one or more metal ions comprise lead ions in an amount of from about 0.05 PPM to about 1 PPM. In some embodiments, the one or more metal ions comprise magnesium ions in an amount of from about 10 PPM to about 300 PPM. In some embodiments, the one or more metal ions comprise manganese ions in an amount of from about 0.1 PPM to about 10 PPM. In some embodiments, the one or more metal ions comprise nickel ions in an amount of from about 0.1 PPM to about 10 PPM. In some embodiments, the one or more metal ions comprise phosphorus ions in an amount of from about 10 PPM to about 300 PPM. In some embodiments, the one or more metal ions comprise potassium ions in an amount of from about 50 PPM to about 3000 PPM. In some embodiments, the one or more metal ions comprise selenium ions in an amount of from about 0.1 PPM to about 1.5 PPM. In some embodiments, the one or more metal ions comprise silver ions in an amount of from about 0.05 PPM to about 0.5 PPM. In some embodiments, the one or more metal ions comprise sodium ions in an amount of from about 10 PPM to about 6000 PPM. In some embodiments, the one or more metal ions comprise tin ions in an amount of from about 0.1 PPM to about 5 PPM. In some embodiments, the one or more metal ions comprise vanadium ions in an amount of from about 0.05 PPM to about 1 PPM. In some embodiments, the one or more metal ions comprise zinc ions in an amount of from about 0.05 PPM to about 20 PPM.

In one aspect, the methods of this invention provide a method of producing fermentation end products comprising contacting a biomass material with a pretreatment and hydrolysis that releases both C6 and C5 sugars and a residual solids portion, adding the C6 and C5 sugar and solids portion to a fermentation process modulated by a biocatalyst, and carrying out the fermentation process until a fermentation end product is produced. In another aspect the biocatalyst is a unicellular microorganism. In a further aspect the biocatalyst consists of a gram⁺ Rhodococcus opacus, a strain of Rhodococcus opacus, or a genetically-modified Rhodococcus opacus microorganism. In some embodiments, the biocatalyst is a yeast microorganism or a modified yeast microorganism. In some embodiments, the microorganism is a gram⁺ or a gram⁻ microorganism. In some embodiments, the microorganism is a modified gram⁺ or a gram⁻ microorganism. In some embodiments, the microorganism is selected from the group consisting of a Rhodococcus strain, a Clostridium strain, a Trichoderma strain, a Saccharomyces strain, and a Zymomonas strain. In a further embodiment, the fermentation process is carried out for 1 to 200 hours. In some embodiments, the biomass is selected from the group consisting of corn stover, sorghum, corncobs, corn mash, sugarcane, bagasse, lignocellulosic, hemicellulosic material, algae, fruit peels, seed hulls, oat hulls, rice hulls, modified crop plants, pectin containing material, starch, wood, algae, distiller's grains, switchgrass, food waste, municipal sewage waste, paper, and paper pulp sludge. In some embodiments, the biomass material is pretreated by acid, steam explosion, hot water treatment, alkali, catalase, or a detoxifying or chelating agent. In some embodiments, the fermentation end-product is butanol, ethanol, propanol, or TAG. In some embodiments, the fermentation end-product is an organic chemical product.

In another aspect of this invention, methods are provided for producing fermentation end-products comprising contacting a biomass material with a pretreatment and hydrolysis that releases both C6 and C5 sugars and residuals, concentrating the C6 and C5 sugars and residuals, adding the C6 and C5 sugar and residuals to a fermentation broth and a biocatalyst, and fermenting all for a time to produce an increase in growth rate of the biocatalyst compared to the same biocatalyst when fermenting a purified C6 and C5 sugar stream containing no microbial nutrients. In some embodiments, the biocatalyst is a unicellular microorganism. In some embodiments, the biocatalyst is selected from a group consisting of a gram⁺ Rhodococcus opacus, a strain of Rhodococcus opacus, or a genetically-modified Rhodococcus opacus microorganism. In some embodiments, the biocatalyst is a yeast microorganism. In some embodiments, the biocatalyst is a modified yeast microorganism. In some embodiments, the microorganism is a gram⁺ or a gram⁻ microorganism. In some embodiments, the microorganism is a modified gram⁺ or a gram⁻ microorganism. In some embodiments, the microorganism is selected from the group consisting of a Rhodococcus strain, a Clostridium strain, a Trichoderma strain, a Saccharomyces strain, and a Zymomonas strain. In some embodiments, the fermentation process is carried out for 1 to 200 hours. In some embodiments, the biomass is selected from the group consisting of corn stover, sorghum, corncobs, corn mash, sugarcane, bagasse, lignocellulosic, hemicellulosic material, algae, fruit peels, seed hulls, oat hulls, rice hulls, modified crop plants, pectin containing material, starch, wood, algae, distiller's grains, switchgrass, food waste, municipal sewage waste, paper, and paper pulp sludge. In some embodiments, the biomass material is pretreated by acid, steam explosion, hot water treatment, alkali, catalase, or a detoxifying or chelating agents. In some embodiments, the fermentation end-product is butanol, ethanol, propanol, or TAG. In some embodiments, the fermentation end-product is an organic chemical product.

In a further aspect of this invention is provided a method of reducing the amount of defined growth media in a fermentation process comprising: pretreating biomass, hydrolyzing said biomass to produce C5 and C6 sugars in a solution with residual nutrients, and replacing all or a portion of defined growth medium for a fermenting microorganism with residual nutrients during a fermentation process. In some embodiments, the fermenting organism is a unicellular microorganism. In some embodiments, the fermenting organism is selected from a group consisting of a gram⁺ Rhodococcus opacus, a strain of Rhodococcus opacus, or a genetically-modified Rhodococcus opacus microorganism. In some embodiments, the fermenting organism is a yeast microorganism. In some embodiments, the fermenting organism is a modified yeast microorganism. In some embodiments, the fermenting microorganism is a gram⁺ or a gram⁻ microorganism. In some embodiments, the fermenting microorganism is a modified gram⁺ or a gram⁻ microorganism. In some embodiments, the solution comprising C5 and C6 sugars modulates said microorganism to produce at least 10% more fermentation end product than the medium. In some embodiments, the solution comprising C5 and C6 sugars modulates the microorganism to produce at least 25% more fermentation end product than the medium. In some embodiments, the solution comprising C5 and C6 sugars modulates the microorganism to produce at least 50% more fermentation end product than the medium. In some embodiments, the solution comprising C5 and C6 sugars modulates the microorganism to produce at least 60% more fermentation end product than the medium. In some embodiments, the solution comprising C5 and C6 sugars modulates the microorganism to produce at least 70% more fermentation end product than the medium. In some embodiments, the solution comprising C5 and C6 sugars modulates the microorganism to produce at least 80% more fermentation end product than the medium. In some embodiments, the solution comprising C5 and C6 sugars modulates the microorganism to produce at least 90% more fermentation end product than the medium. In some embodiments, the solution comprising C5 and C6 sugars modulates the microorganism to produce at least twice as much fermentation end product than the medium.

In a further aspect of this invention a method is provided of producing a TAG fermentation end product, comprising contacting a microorganism with a solution comprising a composition of C5 and C6 sugars and residual nutrients from pretreatment and hydrolysis of biomass, wherein the solution causes the microorganism to produce more of TAG fermentation end product than the microorganism would produce without the solution. In some embodiments, the microorganism is a Rhodococcus microorganism. In some embodiments, the microorganism is a Rhodococcus opacus strain. In some embodiments, the biomass is selected from the group consisting of corn stover, sorghum, bagasse, lignocellulosic, hemicellulosic material, algae, fruit peels, oat hulls, modified crop plants, pectin containing material, starch, wood, algae, distiller's grains, switchgrass, municipal waste, paper, and paper pulp sludge. In some embodiments, the biomass is pretreated by acid, steam explosion, hot water treatment, alkali, catalase, or a detoxifying, flocculating, or chelating agent.

In another aspect of this invention is provided a composition that improves yields of fermentation end product during microbial fermentation of biomass comprising C5 and C6 sugars in a residual nutrient solution. In some embodiments, the composition is further concentrated to increase the mM/L sugar content of the solution. In some embodiments, the mM/L disaccharide content of the solution is decreased relative to the monosaccharide content. In some embodiments, the mM/L sucrose content of the solution is decreased relative to the monosaccharide content.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent that publications and patents or patent applications incorporated by reference contradict the disclosure contained in the specification, the specification is intended to supersede and/or take precedence over any such contradictory material.

### BRIEF DESCRIPTION OF THE DRAWINGS

The novel features disclosed herein are set forth with particularity in the appended claims. A better understanding of the features and advantages will be obtained by reference to the following detailed description that sets forth illustrative embodiments, in which the principles of the disclosure are utilized, and the accompanying drawings of which:

FIG. 1 shows the time course of lipid production on corn stover silage.

FIG. 2 shows TAG productivity by a C5 engineered strain of R. opacus on delignified and enzyme hydrolyzed stover and sorghum.

FIG. 3 shows the overall processes for pretreatment and hydrolysis of biomass and the products that can be incorporated into fermentation.

FIG. 4 shows the increase in glycerol production between sugars produced from the pretreatment and hydrolysis of hardwood with and without particulate solids.

FIG. 5 shows the time-course of glycerol production with and without addition of exogenous minerals to saccharide solutions produced by the pretreatment and hydrolysis of wheat straw.

FIG. 6 shows the time-course of glycerol production with and without addition of exogenous particulate solids to saccharide solutions produced by the pretreatment and hydrolysis of wheat straw.

FIG. 7 shows the increase in glycerol production between pure glucose/xylose solutions with and without addition of exogenous particulates produced during the pretreatment and hydrolysis of oat hulls.

FIG. 8 shows that more glycerol is produced from saccharide solutions containing particulate solids than in saccharide solutions where the particulate solids content was reduced. The saccharide solutions were produced from the pretreatment and hydrolysis of oat hulls

### DETAILED DESCRIPTION

As used in the specification and the appended claims, the singular forms “a,” “an” and “the” include plural referents unless the context clearly dictates otherwise. Thus, for example, reference to “a purified monomer” includes mixtures of two or more purified monomers. The term “comprising” as used herein is synonymous with “including,” “containing,” or “characterized by,” and is inclusive or open-ended and does not exclude additional, unrecited elements or method steps.

“About” means a referenced numeric indication plus or minus 10% of that referenced numeric indication. For example, the term about 4 would include a range of 3.6 to 4.4. All numbers expressing quantities of ingredients, reaction conditions, and so forth used in the specification are to be understood as being modified in all instances by the term “about.” Accordingly, unless indicated to the contrary, the numerical parameters set forth herein are approximations that can vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Wherever the phrase “for example,” “such as,” “including” and the like are used herein, the phrase “and without limitation” is understood to follow unless explicitly stated otherwise. Therefore, “for example ethanol production” means “for example and without limitation ethanol production.”

In this specification and in the claims that follow, reference will be made to a number of terms which shall be defined to have the following meanings

### DEFINITIONS

“Optional” or “optionally” means that the subsequently described event or circumstance may or may not occur, and that the description includes instances where said event or circumstance occurs and instances where it does not. For example, the phrase “the medium can optionally contain glucose” means that the medium may or may not contain glucose as an ingredient and that the description includes both media containing glucose and media not containing glucose.

Unless characterized otherwise, technical and scientific terms used herein have the same meaning as commonly understood by one of ordinary skill in the art.

“Fermentive end-product” and fermentation end-product are used interchangeably herein to include biofuels, chemicals, compounds suitable as liquid fuels, gaseous fuels, triacylglycerols (TAGs), reagents, chemical feedstocks, chemical additives, processing aids, food additives, bioplastiks and precursors to bioplastiks, and other products. Examples of fermentive end-products include but are not limited to 1,4 diacids (succinic, fumaric and malic), 2,5 furan dicarboxylic acid, 3 hydroxy propionic acid, aspartic acid, glucaric acid, glutamic acid, itaconic acid, levulinic acid, 3-hydroxybutyrolactone, glycerol, sorbitol, xylitol/arabinitol, butanediol, butanol, methane, methanol, ethane, ethene, ethanol, n-propane, 1-propene, 1-propanol, propanal, acetone, propionate, n-butane, 1-butene, 1-butanol, butanal, butanoate, isobutanal, isobutanol, 2-methylbutanal, 2-methylbutanol, 3-methylbutanal, 3-methylbutanol, 2-butene, 2-butanol, 2-butanone, 2,3-butanediol, 3-hydroxy-2-butanone, 2,3-butanedione, ethylbenzene, ethenylbenzene, 2-phenylethanol, phenylacetaldehyde, 1-phenylbutane, 4-phenyl-1-butene, 4-phenyl-2-butene, 1-phenyl-2-butene, 1-phenyl-2-butanol, 4-phenyl-2-butanol, 1-phenyl-2-butanone, 4-phenyl-2-butanone, 1-phenyl-2,3-butandiol, 1-phenyl-3-hydroxy-2-butanone, 4-phenyl-3-hydroxy-2-butanone, 1-phenyl-2,3-butanedione, n-pentane, ethylphenol, ethenylphenol, 2-(4-hydroxyphenyl)ethanol, 4-hydroxyphenylacetaldehyde, 1-(4-hydroxyphenyl)butane, 4-(4-hydroxyphenyl)-1-butene, 4-(4-hydroxyphenyl)-2-butene, 1-(4-hydroxyphenyl)-1-butene, 1-(4-hydroxyphenyl)-2-butanol, 4-(4-hydroxyphenyl)-2-butanol, 1-(4-hydroxyphenyl)-2-butanone, 4-(4-hydroxyphenyl)-2-butanone, 1-(4-hydroxyphenyl)-2,3-butandiol, 1-(4-hydroxyphenyl)-3-hydroxy-2-butanone, 4-(4-hydroxyphenyl)-3-hydroxy-2-butanone, 1-(4-hydroxyphenyl)-2,3-butanonedione, indolylethane, indolylethene, 2-(indole-3-)ethanol, n-pentane, 1-pentene, 1-pentanol, pentanal, pentanoate, 2-pentene, 2-pentanol, 3-pentanol, 2-pentanone, 3-pentanone, 4-methylpentanal, 4-methylpentanol, 2,3-pentanediol, 2-hydroxy-3-pentanone, 3-hydroxy-2-pentanone, 2,3-pentanedione, 2-methylpentane, 4-methyl-1-pentene, 4-methyl-2-pentene, 4-methyl-3-pentene, 4-methyl-2-pentanol, 2-methyl-3-pentanol, 4-methyl-2-pentanone, 2-methyl-3-pentanone, 4-methyl-2,3-pentanediol, 4-methyl-2-hydroxy-3-pentanone, 4-methyl-3-hydroxy-2-pentanone, 4-methyl-2,3-pentanedione, 1-phenylpentane, 1-phenyl-1-pentene, 1-phenyl-2-pentene, 1-phenyl-3-pentene, 1-phenyl-2-pentanol, 1-phenyl-3-pentanol, 1-phenyl-2-pentanone, 1-phenyl-3-pentanone, 1-phenyl-2,3-pentanediol, 1-phenyl-2-hydroxy-3-pentanone, 1-phenyl-3-hydroxy-2-pentanone, 1-phenyl-2,3-pentanedione, 4-methyl-1-phenylpentane, 4-methyl-1-phenyl-1-pentene, 4-methyl-1-phenyl-2-pentene, 4-methyl-1-phenyl-3-pentene, 4-methyl-1-phenyl-3-pentanol, 4-methyl-1-phenyl-2-pentanol, 4-methyl-1-phenyl-3-pentanone, 4-methyl-1-phenyl-2-pentanone, 4-methyl-1-phenyl-2,3-pentanediol, 4-methyl-1-phenyl-2,3-pentanedione, 4-methyl-1-phenyl-3-hydroxy-2-pentanone, 4-methyl-1-phenyl-2-hydroxy-3-pentanone, 1-(4-hydroxyphenyl)pentane, 1-(4-hydroxyphenyl)-1-pentene, 1-(4-hydroxyphenyl)-2-pentene, 1-(4-hydroxyphenyl)-3-pentene, 1-(4-hydroxyphenyl)-2-pentanol, 1-(4-hydroxyphenyl)-3-pentanol, 1-(4-hydroxyphenyl)-2-pentanone, 1-(4-hydroxyphenyl)-3-pentanone, 1-(4-hydroxyphenyl)-2,3-pentanediol, 1-(4-hydroxyphenyl)-2-hydroxy-3-pentanone, 1-(4-hydroxyphenyl)-3-hydroxy-2-pentanone, 1-(4-hydroxyphenyl)-2,3-pentanedione, 4-methyl-1-(4-hydroxyphenyl)pentane, 4-methyl-1-(4-hydroxyphenyl)-2-pentene, 4-methyl-1-(4-hydroxyphenyl)-3-pentene, 4-methyl-1-(4-hydroxyphenyl)-1-pentene, 4-methyl-1-(4-hydroxyphenyl)-3-pentanol, 4-methyl-1-(4-hydroxyphenyl)-2-pentanol, 4-methyl-1-(4-hydroxyphenyl)-3-pentanone, 4-methyl-1-(4-hydroxyphenyl)-2-pentanone, 4-methyl-1-(4-hydroxyphenyl)-2,3-pentanediol, 4-methyl-1-(4-hydroxyphenyl)-2,3-pentanedione, 4-methyl-1-(4-hydroxyphenyl)-3-hydroxy-2-pentanone, 4-methyl-1-(4-hydroxyphenyl)-2-hydroxy-3-pentanone, 1-indole-3-pentane, 1-(indole-3)-1-pentene, 1-(indole-3)-2-pentene, 1-(indole-3)-3-pentene, 1-(indole-3)-2-pentanol, 1-(indole-3)-3-pentanol, 1-(indole-3)-2-pentanone, 1-(indole-3)-3-pentanone, 1-(indole-3)-2,3-pentanediol, 1-(indole-3)-2-hydroxy-3-pentanone, 1-(indole-3)-3-hydroxy-2-pentanone, 1-(indole-3)-2,3-pentanedione, 4-methyl-1-(indole-3-)pentane, 4-methyl-1-(indole-3)-2-pentene, 4-methyl-1-(indole-3)-3-pentene, 4-methyl-1-(indole-3)-1-pentene, 4-methyl-2-(indole-3)-3-pentanol, 4-methyl-1-(indole-3)-2-pentanol, 4-methyl-1-(indole-3)-3-pentanone, 4-methyl-1-(indole-3)-2-pentanone, 4-methyl-1-(indole-3)-2,3-pentanediol, 4-methyl-1-(indole-3)-2,3-pentanedione, 4-methyl-1-(indole-3)-3-hydroxy-2-pentanone, 4-methyl-1-(indole-3)-2-hydroxy-3-pentanone, n-hexane, 1-hexene, 1-hexanol, hexanal, hexanoate, 2-hexene, 3-hexene, 2-hexanol, 3-hexanol, 2-hexanone, 3-hexanone, 2,3-hexanediol, 2,3-hexanedione, 3,4-hexanediol, 3,4-hexanedione, 2-hydroxy-3-hexanone, 3-hydroxy-2-hexanone, 3-hydroxy-4-hexanone, 4-hydroxy-3-hexanone, 2-methylhexane, 3-methylhexane, 2-methyl-2-hexene, 2-methyl-3-hexene, 5-methyl-1-hexene, 5-methyl-2-hexene, 4-methyl-1-hexene, 4-methyl-2-hexene, 3-methyl-3-hexene, 3-methyl-2-hexene, 3-methyl-1-hexene, 2-methyl-3-hexanol, 5-methyl-2-hexanol, 5-methyl-3-hexanol, 2-methyl-3-hexanone, 5-methyl-2-hexanone, 5-methyl-3-hexanone, 2-methyl-3,4-hexanediol, 2-methyl-3,4-hexanedione, 5-methyl-2,3-hexanediol, 5-methyl-2,3-hexanedione, 4-methyl-2,3-hexanediol, 4-methyl-2,3-hexanedione, 2-methyl-3-hydroxy-4-hexanone, 2-methyl-4-hydroxy-3-hexanone, 5-methyl-2-hydroxy-3-hexanone, 5-methyl-3-hydroxy-2-hexanone, 4-methyl-2-hydroxy-3-hexanone, 4-methyl-3-hydroxy-2-hexanone, 2,5-dimethylhexane, 2,5-dimethyl-2-hexene, 2,5-dimethyl-3-hexene, 2,5-dimethyl-3-hexanol, 2,5-dimethyl-3-hexanone, 2,5-dimethyl-3,4-hexanediol, 2,5-dimethyl-3,4-hexanedione, 2,5-dimethyl-3-hydroxy-4-hexanone, 5-methyl-1-phenylhexane, 4-methyl-1-phenylhexane, 5-methyl-1-phenyl-1-hexene, 5-methyl-1-phenyl-2-hexene, 5-methyl-1-phenyl-3-hexene, 4-methyl-1-phenyl-1-hexene, 4-methyl-1-phenyl-2-hexene, 4-methyl-1-phenyl-3-hexene, 5-methyl-1-phenyl-2-hexanol, 5-methyl-1-phenyl-3-hexanol, 4-methyl-1-phenyl-2-hexanol, 4-methyl-1-phenyl-3-hexanol, 5-methyl-1-phenyl-2-hexanone, 5-methyl-1-phenyl-3-hexanone, 4-methyl-1-phenyl-2-hexanone, 4-methyl-1-phenyl-3-hexanone, 5-methyl-1-phenyl-2,3-hexanediol, 4-methyl-1-phenyl-2,3-hexanediol, 5-methyl-1-phenyl-3-hydroxy-2-hexanone, 5-methyl-1-phenyl-2-hydroxy-3-hexanone, 4-methyl-1-phenyl-3-hydroxy-2-hexanone, 4-methyl-1-phenyl-2-hydroxy-3-hexanone, 5-methyl-1-phenyl-2,3-hexanedione, 4-methyl-1-phenyl-2,3-hexanedione, 4-methyl-1-(4-hydroxyphenyl)hexane, 5-methyl-1-(4-hydroxyphenyl)-1-hexene, 5-methyl-1-(4-hydroxyphenyl)-2-hexene, 5-methyl-1-(4-hydroxyphenyl)-3-hexene, 4-methyl-1-(4-hydroxyphenyl)-1-hexene, 4-methyl-1-(4-hydroxyphenyl)-2-hexene, 4-methyl-1-(4-hydroxyphenyl)-3-hexene, 5-methyl-1-(4-hydroxyphenyl)-2-hexanol, 5-methyl-1-(4-hydroxyphenyl)-3-hexanol, 4-methyl-1-(4-hydroxyphenyl)-2-hexanol, 4-methyl-1-(4-hydroxyphenyl)-3-hexanol, 5-methyl-1-(4-hydroxyphenyl)-2-hexanone, 5-methyl-1-(4-hydroxyphenyl)-3-hexanone, 4-methyl-1-(4-hydroxyphenyl)-2-hexanone, 4-methyl-1-(4-hydroxyphenyl)-3-hexanone, 5-methyl-1-(4-hydroxyphenyl)-2,3-hexanediol, 4-methyl-1-(4-hydroxyphenyl)-2,3-hexanediol, 5-methyl-1-(4-hydroxyphenyl)-3-hydroxy-2-hexanone, 5-methyl-1-(4-hydroxyphenyl)-2-hydroxy-3-hexanone, 4-methyl-1-(4-hydroxyphenyl)-3-hydroxy-2-hexanone, 4-methyl-1-(4-hydroxyphenyl)-2-hydroxy-3-hexanone, 5-methyl-1-(4-hydroxyphenyl)-2,3-hexanedione, 4-methyl-1-(4-hydroxyphenyl)-2,3-hexanedione, 4-methyl-1-(indole-3-)hexane, 5-methyl-1-(indole-3)-1-hexene, 5-methyl-1-(indole-3)-2-hexene, 5-methyl-1-(indole-3)-3-hexene, 4-methyl-1-(indole-3)-1-hexene, 4-methyl-1-(indole-3)-2-hexene, 4-methyl-1-(indole-3)-3-hexene, 5-methyl-1-(indole-3)-2-hexanol, 5-methyl-1-(indole-3)-3-hexanol, 4-methyl-1-(indole-3)-2-hexanol, 4-methyl-1-(indole-3)-3-hexanol, 5-methyl-1-(indole-3)-2-hexanone, 5-methyl-1-(indole-3)-3-hexanone, 4-methyl-1-(indole-3)-2-hexanone, 4-methyl-1-(indole-3)-3-hexanone, 5-methyl-1-(indole-3)-2,3-hexanediol, 4-methyl-1-(indole-3)-2,3-hexanediol, 5-methyl-1-(indole-3)-3-hydroxy-2-hexanone, 5-methyl-1-(indole-3)-2-hydroxy-3-hexanone, 4-methyl-1-(indole-3)-3-hydroxy-2-hexanone, 4-methyl-1-(indole-3)-2-hydroxy-3-hexanone, 5-methyl-1-(indole-3)-2,3-hexanedione, 4-methyl-1-(indole-3)-2,3-hexanedione, n-heptane, 1-heptene, 1-heptanol, heptanal, heptanoate, 2-heptene, 3-heptene, 2-heptanol, 3-heptanol, 4-heptanol, 2-heptanone, 3-heptanone, 4-heptanone, 2,3-heptanediol, 2,3-heptanedione, 3,4-heptanediol, 3,4-heptanedione, 2-hydroxy-3-heptanone, 3-hydroxy-2-heptanone, 3-hydroxy-4-heptanone, 4-hydroxy-3-heptanone, 2-methylheptane, 3-methylheptane, 6-methyl-2-heptene, 6-methyl-3-heptene, 2-methyl-3-heptene, 2-methyl-2-heptene, 5-methyl-2-heptene, 5-methyl-3-heptene, 3-methyl-3-heptene, 2-methyl-3-heptanol, 2-methyl-4-heptanol, 6-methyl-3-heptanol, 5-methyl-3-heptanol, 3-methyl-4-heptanol, 2-methyl-3-heptanone, 2-methyl-4-heptanone, 6-methyl-3-heptanone, 5-methyl-3-heptanone, 3-methyl-4-heptanone, 2-methyl-3,4-heptanediol, 2-methyl-3,4-heptanedione, 6-methyl-3,4-heptanediol, 6-methyl-3,4-heptanedione, 5-methyl-3,4-heptanediol, 5-methyl-3,4-heptanedione, 2-methyl-3-hydroxy-4-heptanone, 2-methyl-4-hydroxy-3-heptanone, 6-methyl-3-hydroxy-4-heptanone, 6-methyl-4-hydroxy-3-heptanone, 5-methyl-3-hydroxy-4-heptanone, 5-methyl-4-hydroxy-3-heptanone, 2,6-dimethylheptane, 2,5-dimethylheptane, 2,6-dimethyl-2-heptene, 2,6-dimethyl-3-heptene, 2,5-dimethyl-2-heptene, 2,5-dimethyl-3-heptene, 3,6-dimethyl-3-heptene, 2,6-dimethyl-3-heptanol, 2,6-dimethyl-4-heptanol, 2,5-dimethyl-3-heptanol, 2,5-dimethyl-4-heptanol, 2,6-dimethyl-3,4-heptanediol, 2,6-dimethyl-3,4-heptanedione, 2,5-dimethyl-3,4-heptanediol, 2,5-dimethyl-3,4-heptanedione, 2,6-dimethyl-3-hydroxy-4-heptanone, 2,6-dimethyl-4-hydroxy-3-heptanone, 2,5-dimethyl-3-hydroxy-4-heptanone, 2,5-dimethyl-4-hydroxy-3-heptanone, n-octane, 1-octene, 2-octene, 1-octanol, octanal, octanoate, 3-octene, 4-octene, 4-octanol, 4-octanone, 4,5-octanediol, 4,5-octanedione, 4-hydroxy-5-octanone, 2-methyloctane, 2-methyl-3-octene, 2-methyl-4-octene, 7-methyl-3-octene, 3-methyl-3-octene, 3-methyl-4-octene, 6-methyl-3-octene, 2-methyl-4-octanol, 7-methyl-4-octanol, 3-methyl-4-octanol, 6-methyl-4-octanol, 2-methyl-4-octanone, 7-methyl-4-octanone, 3-methyl-4-octanone, 6-methyl-4-octanone, 2-methyl-4,5-octanediol, 2-methyl-4,5-octanedione, 3-methyl-4,5-octanediol, 3-methyl-4,5-octanedione, 2-methyl-4-hydroxy-5-octanone, 2-methyl-5-hydroxy-4-octanone, 3-methyl-4-hydroxy-5-octanone, 3-methyl-5-hydroxy-4-octanone, 2,7-dimethyloctane, 2,7-dimethyl-3-octene, 2,7-dimethyl-4-octene, 2,7-dimethyl-4-octanol, 2,7-dimethyl-4-octanone, 2,7-dimethyl-4,5-octanediol, 2,7-dimethyl-4,5-octanedione, 2,7-dimethyl-4-hydroxy-5-octanone, 2,6-dimethyloctane, 2,6-dimethyl-3-octene, 2,6-dimethyl-4-octene, 3,7-dimethyl-3-octene, 2,6-dimethyl-4-octanol, 3,7-dimethyl-4-octanol, 2,6-dimethyl-4-octanone, 3,7-dimethyl-4-octanone, 2,6-dimethyl-4,5-octanediol, 2,6-dimethyl-4,5-octanedione, 2,6-dimethyl-4-hydroxy-5-octanone, 2,6-dimethyl-5-hydroxy-4-octanone, 3,6-dimethyloctane, 3,6-dimethyl-3-octene, 3,6-dimethyl-4-octene, 3,6-dimethyl-4-octanol, 3,6-dimethyl-4-octanone, 3,6-dimethyl-4,5-octanediol, 3,6-dimethyl-4,5-octanedione, 3,6-dimethyl-4-hydroxy-5-octanone, n-nonane, 1-nonene, 1-nonanol, nonanal, nonanoate, 2-methylnonane, 2-methyl-4-nonene, 2-methyl-5-nonene, 8-methyl-4-nonene, 2-methyl-5-nonanol, 8-methyl-4-nonanol, 2-methyl-5-nonanone, 8-methyl-4-nonanone, 8-methyl-4,5-nonanediol, 8-methyl-4,5-nonanedione, 8-methyl-4-hydroxy-5-nonanone, 8-methyl-5-hydroxy-4-nonanone, 2,8-dimethylnonane, 2,8-dimethyl-3-nonene, 2,8-dimethyl-4-nonene, 2,8-dimethyl-5-nonene, 2,8-dimethyl-4-nonanol, 2,8-dimethyl-5-nonanol, 2,8-dimethyl-4-nonanone, 2,8-dimethyl-5-nonanone, 2,8-dimethyl-4,5-nonanediol, 2,8-dimethyl-4,5-nonanedione, 2,8-dimethyl-4-hydroxy-5-nonanone, 2,8-dimethyl-5-hydroxy-4-nonanone, 2,7-dimethylnonane, 3,8-dimethyl-3-nonene, 3,8-dimethyl-4-nonene, 3,8-dimethyl-5-nonene, 3,8-dimethyl-4-nonanol, 3,8-dimethyl-5-nonanol, 3,8-dimethyl-4-nonanone, 3,8-dimethyl-5-nonanone, 3,8-dimethyl-4,5-nonanediol, 3,8-dimethyl-4,5-nonanedione, 3,8-dimethyl-4-hydroxy-5-nonanone, 3,8-dimethyl-5-hydroxy-4-nonanone, n-decane, 1-decene, 1-decanol, decanoate, 2,9-dimethyldecane, 2,9-dimethyl-3-decene, 2,9-dimethyl-4-decene, 2,9-dimethyl-5-decanol, 2,9-dimethyl-5-decanone, 2,9-dimethyl-5,6-decanediol, 2,9-dimethyl-6-hydroxy-5-decanone, 2,9-dimethyl-5,6-decanedionen-undecane, 1-undecene, 1-undecanol, undecanal. undecanoate, n-dodecane, 1-dodecene, 1-dodecanol, dodecanal, dodecanoate, n-dodecane, 1-decadecene, n-tridecane, 1-tridecene, 1-tridecanol, tridecanal, tridecanoate, n-tetradecane, 1-tetradecene, 1-tetradecanol, tetradecanal, tetradecanoate, n-pentadecane, 1-pentadecene, 1-pentadecanol, pentadecanal, pentadecanoate, n-hexadecane, 1-hexadecene, 1-hexadecanol, hexadecanal, hexadecanoate, n-heptadecane, 1-heptadecene, 1-heptadecanol, heptadecanal, heptadecanoate, n-octadecane, 1-octadecene, 1-octadecanol, octadecanal, octadecanoate, n-nonadecane, 1-nonadecene, 1-nonadecanol, nonadecanal, nonadecanoate, eicosane, 1-eicosene, 1-eicosanol, eicosanal, eicosanoate, 3-hydroxy propanal, 1,3-propanediol, 4-hydroxybutanal, 1,4-butanediol, 3-hydroxy-2-butanone, 2,3-butandiol, 1,5-pentane diol, homocitrate, homoisocitorate, b-hydroxy adipate, glutarate, glutarsemialdehyde, glutaraldehyde, 2-hydroxy-1-cyclopentanone, 1,2-cyclopentanediol, cyclopentanone, cyclopentanol, (S)-2-acetolactate, (R)-2,3-Dihydroxy-isovalerate, 2-oxoisovalerate, isobutyryl-CoA, isobutyrate, isobutyraldehyde, 5-amino pentaldehyde, 1,10-diaminodecane, 1,10-diamino-5-decene, 1,10-diamino-5-hydroxydecane, 1,10-diamino-5-decanone, 1,10-diamino-5,6-decanediol, 1,10-diamino-6-hydroxy-5-decanone, phenylacetoaldehyde, 1,4-diphenylbutane, 1,4-diphenyl-1-butene, 1,4-diphenyl-2-butene, 1,4-diphenyl-2-butanol, 1,4-diphenyl-2-butanone, 1,4-diphenyl-2,3-butanediol, 1,4-diphenyl-3-hydroxy-2-butanone, 1-(4-hydeoxyphenyl)-4-phenylbutane, 1-(4-hydeoxyphenyl)-4-phenyl-1-butene, 1-(4-hydeoxyphenyl)-4-phenyl-2-butene, 1-(4-hydeoxyphenyl)-4-phenyl-2-butanol, 1-(4-hydeoxyphenyl)-4-phenyl-2-butanone, 1-(4-hydeoxyphenyl)-4-phenyl-2,3-butanediol, 1-(4-hydeoxyphenyl)-4-phenyl-3-hydroxy-2-butanone, 1-(indole-3)-4-phenylbutane, 1-(indole-3)-4-phenyl-1-butene, 1-(indole-3)-4-phenyl-2-butene, 1-(indole-3)-4-phenyl-2-butanol, 1-(indole-3)-4-phenyl-2-butanone, 1-(indole-3)-4-phenyl-2,3-butanediol, 1-(indole-3)-4-phenyl-3-hydroxy-2-butanone, 4-hydroxyphenylacetoaldehyde, 1,4-di(4-hydroxyphenyl)butane, 1,4-di(4-hydroxyphenyl)-1-butene, 1,4-di(4-hydroxyphenyl)-2-butene, 1,4-di(4-hydroxyphenyl)-2-butanol, 1,4-di(4-hydroxyphenyl)-2-butanone, 1,4-di(4-hydroxyphenyl)-2,3-butanediol, 1,4-di(4-hydroxyphenyl)-3-hydroxy-2-butanone, 1-(4-hydroxyphenyl)-4-(indole-3-)butane, 1-(4-hydroxyphenyl)-4-(indole-3)-1-butene, 1-di(4-hydroxyphenyl)-4-(indole-3)-2-butene, 1-(4-hydroxyphenyl)-4-(indole-3)-2-butanol, 1-(4-hydroxyphenyl)-4-(indole-3)-2-butanone, 1-(4-hydroxyphenyl)-4-(indole-3)-2,3-butanediol, 1-(4-hydroxyphenyl-4-(indole-3)-3-hydroxy-2-butanone, indole-3-acetoaldehyde, 1,4-di(indole-3-)butane, 1,4-di(indole-3)-1-butene, 1,4-di(indole-3)-2-butene, 1,4-di(indole-3)-2-butanol, 1,4-di(indole-3)-2-butanone, 1,4-di(indole-3)-2,3-butanediol, 1,4-di(indole-3)-3-hydroxy-2-butanone, succinate semialdehyde, hexane-1,8-dicarboxylic acid, 3-hexene-1,8-dicarboxylic acid, 3-hydroxy-hexane-1,8-dicarboxylic acid, 3-hexanone-1,8-dicarboxylic acid, 3,4-hexanediol-1,8-dicarboxylic acid, 4-hydroxy-3-hexanone-1,8-dicarboxylic acid, glycerol, fucoidan, iodine, chlorophyll, carotenoid, calcium, magnesium, iron, sodium, potassium, phosphate, lactic acid, acetic acid, formic acid, isoprenoids, and polyisoprenes, including rubber. Further, such products can include succinic acid, pyruvic acid, enzymes such as cellulases, polysaccharases, lipases, proteases, ligninases, and hemicellulases and may be present as a pure compound, a mixture, or an impure or diluted form.

Fermentation end-products can include polyols or sugar alcohols; for example, methanol, glycol, glycerol, erythritol, threitol, arabitol, xylitol, ribitol, mannitol, sorbitol, dulcitol, fucitol, iditol, inositol, volemitol, isomalt, maltitol, lactitol, and/or polyglycitol.

Fermentation end-products can include fatty acids, oils and fatty acid comprising materials. Examples of fatty acids include, but are not limited to butyric acid, hexanoic acid, octanoic acid, decanoic acid, lauric acid, tridecanoic acid, myristic acid, pentadecanoic acid, palmitic acid, heptadecanoic acid, stearic acid, arachidic acid, heneicosanoic acid, behenic acid, tricosanoic acid, lignoceric acid, (cis-9) myristoleic acid, (cis-10) pentadecinoic acid, (cis-9) palmitoleic acid, (cis-10) heptadecenoate acid, (cis-9) oleic acid, (cis-11) eicosenoic acid, (cis-13) erucic acid, (cis-15) nervonic acid, (cis-9, 12) lonoleic acid, (cis-6, 9, 12) y-linolenic acid, (cis-9, 12,15) linolenic acid, (cis-11, 14) eicosadienoic acid, (cis-8, 11, 14) eicosatrienoic acid, (cis-11, 14, 17) eicosatrienoic acid, (cis-5, 8, 11, 14) arachidonic acid, (cis-5, 8, 11, 14, 17) eicosapentanoic acid, (cis-13, 16) docosadienoic acid, (cis-4, 7, 10, 13, 16, 19) docosahexaenoic acid, (trans-9) methyl elaidate acid, and (trans-9, 12) methyl linoelaidate acid.

The term “fatty acid comprising material” as used herein has its ordinary meaning as known to those skilled in the art and can comprise one or more chemical compounds that include one or more fatty acid moieties as well as derivatives of these compounds and materials that comprise one or more of these compounds. Common examples of compounds that include one or more fatty acid moieties include triacylglycerides, diacylglycerides, monoacylglycerides, phospholipids, lysophospholipids, free fatty acids, fatty acid salts, soaps, fatty acid comprising amides, esters of fatty acids and monohydric alcohols, esters of fatty acids and polyhydric alcohols including glycols (e.g. ethylene glycol, propylene glycol, etc.), esters of fatty acids and polyethylene glycol, esters of fatty acids and polyethers, esters of fatty acids and polyglycol, esters of fatty acids and saccharides, esters of fatty acids with other hydroxyl-containing compounds, etc. A fatty acid comprising material can be one or more of these compounds in an isolated or purified form. It can be a material that includes one or more of these compounds that is combined or blended with other similar or different materials. It can be a material where the fatty acid comprising material occurs with or is provided with other similar or different materials, such as vegetable and animal oils; mixtures of vegetable and animal oils; vegetable and animal oil byproducts; mixtures of vegetable and animal oil byproducts; vegetable and animal wax esters; mixtures, derivatives and byproducts of vegetable and animal wax esters; seeds; processed seeds; seed byproducts; nuts; processed nuts; nut byproducts; animal matter; processed animal matter; byproducts of animal matter; corn; processed corn; corn byproducts; distiller's grains; beans; processed beans; bean byproducts; soy products; lipid containing plant, fish or animal matter; processed lipid containing plant or animal matter; byproducts of lipid containing plant, fish or animal matter; lipid containing microbial material; processed lipid containing microbial material; and byproducts of lipid containing microbial matter. Such materials can be utilized in liquid or solid forms. Solid forms include whole forms, such as cells, beans, and seeds; ground, chopped, slurried, extracted, flaked, milled, etc. The fatty acid portion of the fatty acid comprising compound can be a simple fatty acid, such as one that includes a carboxyl group attached to a substituted or un-substituted alkyl group. The substituted or unsubstituted alkyl group can be straight or branched, saturated or unsaturated. Substitutions on the alkyl group can include hydroxyls, phosphates, halogens, alkoxy, or aryl groups. The substituted or unsubstituted alkyl group can have 7 to 29 carbons and preferably 11 to 23 carbons (e.g., 8 to 30 carbons and preferably 12 to 24 carbons counting the carboxyl group) arranged in a linear chain with or without side chains and/or substitutions. Addition of the fatty acid comprising compound can be by way of adding a material comprising the fatty acid comprising compound.

The term “pH modifier” as used herein has its ordinary meaning as known to those skilled in the art and can include any material that will tend to increase, decrease or hold steady the pH of the broth or medium. A pH modifier can be an acid, a base, a buffer, or a material that reacts with other materials present to serve to raise, lower, or hold steady the pH. In one embodiment, more than one pH modifier can be used, such as more than one acid, more than one base, one or more acid with one or more bases, one or more acids with one or more buffers, one or more bases with one or more buffers, or one or more acids with one or more bases with one or more buffers. In one embodiment, a buffer can be produced in the broth or medium or separately and used as an ingredient by at least partially reacting in acid or base with a base or an acid, respectively. When more than one pH modifiers are utilized, they can be added at the same time or at different times. In one embodiment, one or more acids and one or more bases are combined, resulting in a buffer. In one embodiment, media components, such as a carbon source or a nitrogen source serve as a pH modifier; suitable media components include those with high or low pH or those with buffering capacity. Exemplary media components include acid- or base-hydrolyzed plant polysaccharides having residual acid or base, ammonia fiber explosion (AFEX) treated plant material with residual ammonia, lactic acid, corn steep solids or liquor.

The term “fermentation” as used herein has its ordinary meaning as known to those skilled in the art and can include culturing of a microorganism or group of microorganisms in or on a suitable medium for the microorganisms. The microorganisms can be aerobes, anaerobes, facultative anaerobes, heterotrophs, autotrophs, photoautotrophs, photoheterotrophs, chemoautotrophs, and/or chemoheterotrophs. The microorganisms can be growing aerobically or anaerobically. They can be in any phase of growth, including lag (or conduction), exponential, transition, stationary, death, dormant, vegetative, sporulating, etc.

“Growth phase” is used herein to describe the type of cellular growth that occurs after the “Initiation phase” and before the “Stationary phase” and the “Death phase.” The growth phase is sometimes referred to as the exponential phase or log phase or logarithmic phase.

The term “plant polysaccharide” as used herein has its ordinary meaning as known to those skilled in the art and can comprise one or more polymers of sugars and sugar derivatives as well as derivatives of sugar polymers and/or other polymeric materials that occur in plant matter. Exemplary plant polysaccharides include lignin, cellulose, starch, pectin, and hemicellulose. Others are chitin, sulfonated polysaccharides such as alginic acid, agarose, carrageenan, porphyran, furcelleran and funoran. Generally, the polysaccharide can have two or more sugar units or derivatives of sugar units. The sugar units and/or derivatives of sugar units can repeat in a regular pattern, or otherwise. The sugar units can be hexose units or pentose units, or combinations of these. The derivatives of sugar units can be sugar alcohols, sugar acids, amino sugars, etc. The polysaccharides can be linear, branched, cross-linked, or a mixture thereof. One type or class of polysaccharide can be cross-linked to another type or class of polysaccharide. The concentration of saccharides in a biomass containing plant polysaccharides such as cellulose, hemicellulose, starch, or pectin can be given in terms of monosaccharide equivalents. A monosaccharide equivalent concentration is the concentration of saccharides assuming complete hydrolysis of polysaccharides to monosaccharides.

The term “fermentable sugars” as used herein has its ordinary meaning as known to those skilled in the art and can include one or more sugars and/or sugar derivatives that can be utilized as a carbon source by the microorganism, including monomers, dimers, and polymers of these compounds including two or more of these compounds. In some cases, the organism can break down these polymers, such as by hydrolysis, prior to incorporating the broken down material. Exemplary fermentable sugars include, but are not limited to glucose, dextrose, xylose, arabinose, galactose, mannose, rhamnose, cellobiose, lactose, sucrose, maltose, and fructose.

The term “saccharification” as used herein has its ordinary meaning as known to those skilled in the art and can include conversion of plant polysaccharides to lower molecular weight species that can be utilized by the organism at hand. For some organisms, this would include conversion to monosaccharides, disaccharides, trisaccharides, and oligosaccharides of up to about seven monomer units, as well as similar sized chains of sugar derivatives and combinations of sugars and sugar derivatives. The terms “SSF” and “SHF” are known to those skilled in the art; SSF meaning simultaneous saccharification and fermentation, or the conversion from polysaccharides or oligosaccharides into monosaccharides at the same time and in the same fermentation vessel wherein monosaccharides are converted to another chemical product such as ethanol. “SHF” indicates a physical separation of the polymer hydrolysis or saccharification and fermentation processes.

The term “biomass” as used herein has its ordinary meaning as known to those skilled in the art and can include one or more biological materials that can be converted into a biofuel, chemical or other product. Biomass as used herein is synonymous with the term “feedstock” and includes corn syrup, molasses, silage, agricultural residues (corn stalks, grass, straw, grain hulls, bagasse, etc.), animal waste (manure from cattle, poultry, and hogs), Distillers Dried Solubles (DDS), Distillers Dried Grains (DDG), Condensed Distillers Solubles (CDS), Distillers Wet Grains (DWG), Distillers Dried Grains with Solubles (DDGS), woody materials (wood or bark, sawdust, timber slash, and mill scrap), municipal waste (waste paper, recycled toilet papers, yard clippings, etc.), and energy crops (poplars, willows, switchgrass, alfalfa, prairie bluestem, algae, including macroalgae, etc.). One exemplary source of biomass is plant matter. Plant matter can be, for example, woody plant matter, non-woody plant matter, cellulosic material, lignocellulosic material, hemicellulosic material, carbohydrates, pectin, starch, inulin, fructans, glucans, corn, sugar cane, grasses, switchgrass, sorghum, high biomass sorghum, bamboo, algae and material derived from these. Plants can be in their natural state or genetically modified, e.g., to increase the cellulosic or hemicellulosic portion of the cell wall, or to produce additional exogenous or endogenous enzymes to increase the separation of cell wall components. Plant matter can also include plant cell culture or plant cell tissue culture. Plant matter can be further described by reference to the chemical species present, such as proteins, polysaccharides and oils. Polysaccharides include polymers of various monosaccharides and derivatives of monosaccharides including glucose, fructose, lactose, galacturonic acid, rhamnose, etc. Plant matter also includes agricultural waste byproducts or side streams such as pomace, corn steep liquor, corn steep solids, distillers grains, peels, pits, fermentation waste, straw, lumber, sewage, garbage and food leftovers. Peels can be citrus which include, but are not limited to, tangerine peel, grapefruit peel, orange peel, tangerine peel, lime peel and lemon peel. These materials can come from farms, forestry, industrial sources, households, etc. Another non-limiting example of biomass is animal matter, including, for example milk, meat, fat, animal processing waste, and animal waste. “Feedstock” is frequently used to refer to biomass being used for a process, such as those described herein.

“Broth” is used herein to refer to inoculated medium at any stage of growth, including the point immediately after inoculation and the period after any or all cellular activity has ceased and can include the material after post-fermentation processing. It includes the entire contents of the combination of soluble and insoluble matter, suspended matter, cells and medium, as appropriate.

The term “productivity” as used herein has its ordinary meaning as known to those skilled in the art and can include the mass of a material of interest produced in a given time in a given volume. Units can be, for example, grams per liter-hour, or some other combination of mass, volume, and time. In fermentation, productivity is frequently used to characterize how fast a product can be made within a given fermentation volume. The volume can be referenced to the total volume of the fermentation vessel, the working volume of the fermentation vessel, or the actual volume of broth being fermented. The context of the phrase will indicate the meaning intended to one of skill in the art. Productivity is different from “titer” in that productivity includes a time term, and titer is analogous to concentration. Titer and Productivity can generally be measured at any time during the fermentation, such as at the beginning, the end, or at some intermediate time, with titer relating the amount of a particular material present or produced at the point in time of interest and the productivity relating the amount of a particular material produced per liter in a given amount of time. The amount of time used in the productivity determination can be from the beginning of the fermentation or from some other time, and go to the end of the fermentation, such as when no additional material is produced or when harvest occurs, or some other time as indicated by the context of the use of the term. “Overall productivity” refers to the productivity determined by utilizing the final titer and the overall fermentation time.

“Titer” refers to the amount of a particular material present in a fermentation broth. It is similar to concentration and can refer to the amount of material made by the organism in the broth from all fermentation cycles, or the amount of material made in the current fermentation cycle or over a given period of time, or the amount of material present from whatever source, such as produced by the organism or added to the broth. Frequently, the titer of soluble species will be referenced to the liquid portion of the broth, with insolubles removed, and the titer of insoluble species will be referenced to the total amount of broth with insoluble species being present, however, the titer of soluble species can be referenced to the total broth volume and the titer of insoluble species can be referenced to the liquid portion, with the context indicating the which system is used with both reference systems intended in some cases. Frequently, the value determined referenced to one system will be the same or a sufficient approximation of the value referenced to the other.

“Concentration” when referring to material in the broth generally refers to the amount of a material present from all sources, whether made by the organism or added to the broth. Concentration can refer to soluble species or insoluble species, and is referenced to either the liquid portion of the broth or the total volume of the broth, as for “titer.”

The term “biocatalyst” as used herein has its ordinary meaning as known to those skilled in the art and can include one or more enzymes and/or microorganisms, including solutions, suspensions, and mixtures of enzymes and microorganisms. In some contexts this word will refer to the possible use of either enzymes or microorganisms to serve a particular function, in other contexts the word will refer to the combined use of the two, and in other contexts the word will refer to only one of the two. The context of the phrase will indicate the meaning intended to one of skill in the art. For example, a biocatalyst can be a fermenting microorganism. The term biocatalyst includes fermenting microorganisms such as yeast, bacteria, algae, and plant cells.

The terms “conversion efficiency” or “yield” as used herein have their ordinary meaning as known to those skilled in the art and can include the mass of product made from a mass of substrate. The term can be expressed as a percentage yield of the product from a starting mass of substrate. For the production of ethanol from glucose, the net reaction is generally accepted as:

C₆H₁₂O₆→2C₂H₅OH+2CO₂

and the theoretical maximum conversion efficiency, or yield, is 51% (wt.). Frequently, the conversion efficiency will be referenced to the theoretical maximum, for example, “80% of the theoretical maximum.” In the case of conversion of glucose to ethanol, this statement would indicate a conversion efficiency of 41% (wt.). The context of the phrase will indicate the substrate and product intended to one of skill in the art.

“Pretreatment” or “pretreated” is used herein to refer to any mechanical, chemical, thermal, biochemical process or combination of these processes whether in a combined step or performed sequentially, that achieves disruption or expansion of the biomass so as to render the biomass more susceptible to attack by enzymes and/or microbes. In one embodiment, pretreatment includes removal or disruption of lignin so as to make the cellulose and hemicellulose polymers in the plant biomass more available to cellulolytic enzymes and/or microbes, for example, by treatment with acid or base. In one embodiment, pretreatment includes disruption or expansion of cellulosic and/or hemicellulosic material. Steam explosion, and ammonia fiber expansion (or explosion) (AFEX) are well known thermal/chemical techniques. Hydrolysis, including methods that utilize acids, bases, and/or enzymes can be used. Other thermal, chemical, biochemical, enzymatic techniques can also be used.

“Fed-batch” or “fed-batch fermentation” is used herein to include methods of culturing microorganisms where nutrients, other medium components, or biocatalysts (including, for example, enzymes, fresh organisms, extracellular broth, genetically modified plants and/or organisms, etc.) are supplied to the fermentor during cultivation, but culture broth is not harvested from the fermentor until the end of the fermentation, although it can also include “self seeding” or “partial harvest” techniques where a portion of the fermentor volume is harvested and then fresh medium is added to the remaining broth in the fermentor, with at least a portion of the inoculum being the broth that was left in the fermentor. During a fed-batch fermentation, the broth volume can increase, at least for a period, by adding medium or nutrients to the broth while fermentation organisms are present. Suitable nutrients which can be utilized include those that are soluble, insoluble, and partially soluble, including gasses, liquids and solids. In one embodiment, a fed-batch process is referred to with a phrase such as, “fed-batch with cell augmentation.” This phrase can include an operation where nutrients and cells are added or one where cells with no substantial amount of nutrients are added. The more general phrase “fed-batch” encompasses these operations as well. The context where any of these phrases is used will indicate to one of skill in the art the techniques being considered.

The terms “sugar compounds”, “sugar streams”, “saccharide compounds”, “saccharide streams”, “saccharide solutions” are used interchangeably herein to indicate mostly monosaccharide sugars, dissolved, crystallized, evaporated, or partially dissolved, including but not limited to hexoses and pentoses; sugar alcohols; sugar acids; sugar amines; compounds containing two or more of these linked together directly or indirectly through covalent or ionic bonds; and mixtures thereof. Included within this description are disaccharides; trisaccharides; oligosaccharides; polysaccharides; and sugar chains, branched and/or linear, of any length. A sugar stream can consist of primarily or substantially C6 sugars (e.g., a C6-rich stream), C5 sugars (e.g., a C5-rich stream), or mixtures of both C6 and C5 sugars in varying ratios of said sugars. C6 sugars have a six-carbon molecular backbone and C5 sugars have a five-carbon molecular backbone. Sugar compounds, sugar streams, saccharide compounds, saccharide streams, or saccharide solutions can be produced from the pretreatment and/or hydrolysis of biomass. The biomass can comprise cellulose, hemicellulose, lignocellulose, starch, or a combination thereof. Sugars or sugar streams produced from cellulose, hemicellulose, and/or lignocellulose can be termed “cellulosic-derived saccharides”. Sugars or sugar streams produced from starch can be termed “non-cellulosic-derived saccharides” or “non-cellulosic derived saccharide streams.”

“C5-rich” composition means that one or more steps have been taken to remove at least some of the C6 sugars originally in the composition. For example, a C5-rich composition can include no more than about 50% C6 sugars, no more than about 40% C6 sugars, no more than about 30% C6 sugars, no more than about 20% C6 sugars, no more than about 10% C6 sugars, no more than about 5% C6 sugars, or it can include from about 2% to about 10% C6 sugars by weight. Likewise, a “C6-rich” composition is one in which at least some of the originally-present C5 sugars have been removed. For example, a C6-rich composition can include no more than about 50% C5 sugars, nor more than about 40% C5 sugars, no more than about 30% C5 sugars, no more than about 20% C5 sugars, no more than about 10% C5 sugars, no more than about 5% C5 sugars, or it can include from about 2% to about 10% C5 sugars by weight.

A “liquid” composition may contain solids and a “solids” composition may contain liquids. A liquid composition refers to a composition in which the material is primarily liquid, and a solids composition is one in which the material is primarily solid.

“Gentle Pretreatment” generally refers to the collection of processes upstream of hydrolysis, which result in composition that, when hydrolyzed, produces a fermentable sugar composition. The fermentable sugar composition can be used to enhance a non-cellulosic fermentation process, such as a corn mash fermentation process. In some embodiments, the gentle pretreatment process provides a fermentable sugar composition having a favorable nutrient balance (e.g. plant-derived extracted nutrients, which are part of the composition as a result of the pretreatment process) and/or an amount of toxic compounds (e.g. phenolics and sugar degradation products, organic acids and furans, which inhibit and/or inactivate the performance of enzymes and or fermentation organisms), which is limited such that the resultant fermentable sugar composition can enhance a non-cellulosic fermentation process, such as a corn mash fermentation process. For example, a gentle pretreatment is one that results in a sugar stream that is about 25% (w/v) C6 sugars or more, about 4 g/L hydroxymethyl furfural or less, about 4 g/L furfural or less, about 10 g/L acetic acid or less, about 10 g/L formic acid or less for example as measured by typical HPLC methods referred to herein. (“About X amount of a substance or less” means the same as “no more than about” and includes zero—i.e. includes the possibility that none of that substance is present in the composition.) “Gentle pretreatment” can include one or more of: pre-processing biomass to reduce size and/or create size uniformity; pretreatment itself (process for making cellulose more accessible to hydrolysis); and post-processing steps such as washing steps.

The terms “non-cellulosic” and “sugar- or starch-based” are used interchangeably and have the same meaning. For example “non-cellulosic fermentation process” is used interchangeably and means the same thing as “sugar- and starch-based fermentation process.” Starch is a carbohydrate consisting of consisting of a large number of glucose units joined by glycosidic bonds. The glycosidic bonds are typically the easily hydrolysable alpha glycosidic bonds. This polysaccharide can be produced by all green plants as an energy store. There can be two types of starch molecules: the linear and helical amylose and the branched amylopectin, although amylase can also contain branches.

### Description

The following description and examples illustrate some exemplary embodiments of the disclosure in detail. Those of skill in the art will recognize that there are numerous variations and modifications of this disclosure that are encompassed by its scope. Accordingly, the description of a certain exemplary embodiment should not be deemed to limit the scope of the present disclosure.

### Feedstock and Pretreatment of Feedstock

In one embodiment, the feedstock (biomass) contains cellulosic, hemicellulosic, and/or lignocellulosic material. The feedstock can be derived from agricultural crops, crop residues, trees, woodchips, sawdust, paper, cardboard, grasses, algae, municipal waste and other sources.

Cellulose is a linear polymer of glucose where the glucose units are connected via β(1→4) linkages. Hemicellulose is a branched polymer of a number of sugar monomers including glucose, xylose, mannose, galactose, rhamnose and arabinose, and can have sugar acids such as mannuronic acid and galacturonic acid present as well. Lignin is a cross-linked, racemic macromolecule of mostly p-coumaryl alcohol, conferyl alcohol and sinapyl alcohol. These three polymers occur together in lignocellulosic materials in plant biomass. The different characteristics of the three polymers can make hydrolysis of the combination difficult as each polymer tends to shield the others from enzymatic attack.

In one embodiment, methods are provided for the pretreatment of feedstock used in the fermentation and production of the biofuels and chemicals. The pretreatment steps can include mechanical, thermal, pressure, chemical, thermochemical, and/or biochemical tests pretreatment prior to being used in a bioprocess for the production of fuels and chemicals, but untreated biomass material can be used in the process as well. Mechanical processes can reduce the particle size of the biomass material so that it can be more conveniently handled in the bioprocess and can increase the surface area of the feedstock to facilitate contact with chemicals/biochemicals/biocatalysts. Mechanical processes can also separate one type of biomass material from another. The biomass material can also be subjected to thermal and/or chemical pretreatments to render plant polymers more accessible. Multiple steps of treatment can also be used.

Mechanical processes include, are not limited to, washing, soaking, milling, size reduction, screening, shearing, size classification and density classification processes. Chemical processes include, but are not limited to, bleaching, oxidation, reduction, acid treatment, base treatment, sulfite treatment, acid sulfite treatment, basic sulfite treatment, ammonia treatment, and hydrolysis. Thermal processes include, but are not limited to, sterilization, ammonia fiber expansion or explosion (“AFEX”), steam explosion, holding at elevated temperatures, pressurized or unpressurized, in the presence or absence of water, and freezing. Biochemical processes include, but are not limited to, treatment with enzymes, including enzymes produced by genetically-modified plants, and treatment with microorganisms. Various enzymes that can be utilized include cellulase, amylase, β-glucosidase, xylanase, gluconase, and other polysaccharases; lysozyme; laccase, and other lignin-modifying enzymes; lipoxygenase, peroxidase, and other oxidative enzymes; proteases; and lipases. One or more of the mechanical, chemical, thermal, thermochemical, and biochemical processes can be combined or used separately. Such combined processes can also include those used in the production of paper, cellulose products, microcrystalline cellulose, and cellulosics and can include pulping, kraft pulping, acidic sulfite processing. The feedstock can be a side stream or waste stream from a facility that utilizes one or more of these processes on a biomass material, such as cellulosic, hemicellulosic or lignocellulosic material. Examples include paper plants, cellulosics plants, distillation plants, cotton processing plants, and microcrystalline cellulose plants. The feedstock can also include cellulose-containing or cellulosic containing waste materials. The feedstock can also be biomass materials, such as wood, grasses, corn, starch, or sugar, produced or harvested as an intended feedstock for production of ethanol or other products such as by biocatalysts.

In another embodiment, a method can utilize a pretreatment process disclosed in U.S. patents and patent applications US20040152881, US20040171136, US20040168960, US20080121359, US20060069244, US20060188980, US20080176301, U.S. Pat. No. 5,693,296, U.S. Pat. No. 6,262,313, US20060024801, U.S. Pat. No. 5,969,189, U.S. Pat. No. 6,043,392, US20020038058, U.S. Pat. No. 5,865,898, U.S. Pat. No. 5,865,898, U.S. Pat. No. 6,478,965, U.S. Pat. No. 5,986,133, or US20080280338, each of which is incorporated by reference herein in its entirety

In another embodiment, the AFEX process is be used for pretreatment of biomass. In a preferred embodiment, the AFEX process is used in the preparation of cellulosic, hemicellulosic or lignocellulosic materials for fermentation to ethanol or other products. The process generally includes combining the feedstock with ammonia, heating under pressure, and suddenly releasing the pressure. Water can be present in various amounts. The AFEX process has been the subject of numerous patents and publications.

In another embodiment, the pretreatment of biomass comprises the addition of calcium hydroxide to a biomass to render the biomass susceptible to degradation. Pretreatment comprises the addition of calcium hydroxide and water to the biomass to form a mixture, and maintaining the mixture at a relatively high temperature. Alternatively, an oxidizing agent, selected from the group consisting of oxygen and oxygen-containing gasses, can be added under pressure to the mixture. Examples of carbon hydroxide treatments are disclosed in U.S. Pat. No. 5,865,898 to Holtzapple and S. Kim and M. T. Holtzapple, Bioresource Technology, 96, (2005) 1994, incorporated by reference herein in its entirety.

In one embodiment, pretreatment of biomass comprises dilute acid hydrolysis. Examples of dilute acid hydrolysis treatment are disclosed in T. A. Lloyd and C. E Wyman, Bioresource Technology, (2005) 96, 1967, incorporated by reference herein in its entirety.

In another embodiment, pretreatment of biomass comprises pH controlled liquid hot water treatment. Examples of pH controlled liquid hot water treatments are disclosed in N. Mosier et al., Bioresource Technology, (2005) 96, 1986, incorporated by reference herein in its entirety.

In one embodiment, pretreatment of biomass comprises aqueous ammonia recycle process (ARP). Examples of aqueous ammonia recycle process are described in T. H. Kim and Y. Y. Lee, Bioresource Technology, (2005)96, 2007, incorporated by reference herein in its entirety.

In one embodiment, the above mentioned methods have two steps: a pretreatment step that leads to a wash stream, and an enzymatic hydrolysis step of pretreated-biomass that produces a hydrolysate stream. In the above methods, the pretreatment step can include acid hydrolysis, hot water pretreatment, steam explosion or alkaline reagent based methods (AFEX, ARP, and lime pretreatments). Dilute acid and hot water treatment methods can be used to solubilize all or a portion of the hemicellulose. Methods employing alkaline reagents can be used remove all, most, or a portion of the lignin during the pretreatment step. As a result, the wash stream from the pretreatment step in the former methods contains mostly hemicellulose-based sugars, whereas this stream has mostly lignin for the high-pH methods. The subsequent enzymatic hydrolysis of the residual biomass leads to mixed sugars (C5 and C6) in the alkali based pretreatment methods, while glucose is the major product in the hydrolysate from the low and neutral pH methods. Such a hydrolysate can be referred to as a C6-enriched hydrolysate. In one embodiment, the treated material is additionally treated with catalase or another similar chemical, chelating agents, surfactants, and other compounds to remove impurities or toxic chemicals or further release polysaccharides.

In one embodiment, a saccharide stream or saccharide solution comprising one or more monosaccharides are produced by pretreating and/or hydrolyzing a biomass comprising cellulose, hemicellulose, lignocellulose and/or starch. The biomass can be pretreated according to any of the methods disclosed herein; for example, by dilute acid, hot water treatment, stream explosion, or an alkaline pretreatment. The biomass can be pretreated using a combination of techniques; for example, the biomass can be pretreated using hot water or stream explosion followed by alkaline treatment. The one or more monosaccharides can include C6 and/or C5 monosaccharides. The one or more monosaccharides can be in a C6-enriched hydrolysate (C6 Saccharide Stream). The one or more monosaccharides can be in a C5-enriched hydrolysate (C5 Saccharide Stream). The one or more monosaccharides can comprise both C5 and C6 saccharides (C5+C6 Saccharide Stream). The one or more monosaccharides can include cellulosic-derived monosaccharides. The one or more monosaccharides can include non-cellulosic-derived monosaccharides (e.g., starch-derived monosaccharides). The one or more monosaccharides can include glucose, fructose, galactose, xylose, or any other saccharides.

A C6-enriched hydrolysate (C6 Saccharide Stream) is enriched for C6 saccharides; however, the C6-enriched hydrolysate can comprise C5 saccharides. In one embodiment, less than about 50%, 40%, 30%, 20%, 10%, or 1% of the sugars in the C6-enriched hydrolysate are C5 sugars. In another embodiment, about 0-50%, 0-40%, 0-30%, 0-20%, 0-10%, 0-1%, 0-0.1%, 0.1-50%, 0.1-40%, 0.1-30%, 0.1-20%, 0.1-10%, 0.1-1%, 1-50%, 1-40%, 1-30%, 1-20%, 1-10%, 10-50%, 10-40%, 10-30%, 10-20%, 20-50%, 20-40%, 20-30%, 30-50%, 30-40%, of 40-50% of the sugars in a C6-enriched hydrolysate are C5 sugars. The C6-enriched hydrolysate can comprise one or more cellulosic-derived C6 monosaccharides (e.g., glucose). The C6-enriched hydrolysate can comprise one or more non-cellulosic derived monosaccharides (e.g., starch-derived monosaccharides, e.g., glucose).

A hydrolyzate, saccharide stream, or saccharide solution comprising one or more cellulosic or non-cellulosic derived saccharides can further comprise particulate solids. The particulate solids can be residual solids. The particulate solids (e.g., residual solids) can also be referred to as insoluble solids or suspended solids. The particulate solids (e.g., residual solids) can include cellulose, hemicellulose, lignin, or starch that was unhydrolyzed during pretreatment and hydrolysis of a biomass. The particulate solids (e.g., residual solids) can comprise or further comprise proteins; fats; oils, or a combination thereof. Particulate solids can be added to a saccharide solution. For example, unhydrolyzed cellulose, hemicellulose, lignin, and/or starch can be sequestered or collected from a hydrolyzate and added to another saccharide solution. Such particulate solids can be referred to as exogenous particulate solids.

A hydrolyzate, saccharide stream, or saccharide solution can comprise from about 0% to about 50% w/v particulate solids (e.g., residual solids); for example, about 0-50%, 0-25%, 0-15%, 0-10%, 0-5%, 0-1%, 1-50%, 1-25%, 1-15%, 1-10%, 1-5%, 5-50%, 5-25%, 5-15%, 5-10%, 10-50%, 10-25%, 10-15%, 15-50%, 15-25%, 25-50%, 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% w/v particulate solids (e.g., residual solids). The particulate solids can be exogenously added to the hydrolyzate, saccharide stream, or saccharide solution. The particulate solids can be in the hydrolyzate, saccharide stream, or saccharide solution as a result of the biomass pretreatment and/or hydrolysis used to produce the hydrolyzate, saccharide stream, or saccharide solution.

Particulate solids (e.g., residual, insoluble or suspended solids) in a hydrolyzate, saccharide stream, or saccharide solution can have, for example, particle sizes of from about 1 μM to about 5 mm or larger or smaller. The particulate solids can have particles sizes of about 0.001-5 mm, 0.01-2.5 mm, 0.01-1 mm, 0.01-0.5 mm, 0.01-0.1 mm, 0.01-0.05 mm, 0.05-2.5 mm, 0.05-1 mm, 0.05-0.5 mm, 0.05-0.1 mm, 0.1-2.5 mm, 0.1-1 mm, 0.1-0.5 mm, 0.5-2.5 mm, 0.5-1 mm, 1-2.5 mm, 0.001 mm, 0.002 mm, 0.003 mm, 0.004 mm, 0.005 mm, 0.006 mm, 0.007 mm, 0.008 mm, 0.009 mm, 0.01 mm, 0.02 mm, 0.03 mm, 0.04 mm, 0.05 mm, 0.06 mm, 0.07 mm, 0.08 mm, 0.09 mm, 0.1 mm, 0.11 mm, 0.12 mm, 0.13 mm, 0.14 mm, 0.15 mm, 0.16 mm, 0.17 mm, 0.18 mm, 0.19 mm, 0.2 mm, 0.25 mm, 0.3 mm, 0.35 mm, 0.4 mm, 0.45 mm, 0.5 mm, 0.55 mm, 0.6 mm, 0.65 mm, 0.7 mm, 0.75 mm, 0.8 mm, 0.85 mm, 0.9 mm, 0.95 mm, 1 mm, 1.05 mm, 1.1 mm, 1.15 mm, 1.2 mm, 1.25 mm, 1.3 mm, 1.35 mm, 1.4 mm, 1.45 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.75 mm, 3 mm, 3.25 mm, 3.5 mm, 3.75 mm, 4 mm, 4.5 mm, or 5 mm. The particulate solids can be exogenously added to the hydrolyzate, saccharide stream, or saccharide solution. The particulate solids can be in the hydrolyzate, saccharide stream, or saccharide solution as a result of the biomass pretreatment and/or hydrolysis used to produce the hydrolyzate, saccharide stream, or saccharide solution.

Particulate solids (e.g., residual, insoluble or suspended solids) in a hydrolyzate, saccharide stream, or saccharide solution can have, for example, average particle sizes less than about 5 mm. The particulate solids can have average particle sizes less than about 0.001 mm, 0.002 mm, 0.003 mm, 0.004 mm, 0.005 mm, 0.006 mm, 0.007 mm, 0.008 mm, 0.009 mm, 0.01 mm, 0.02 mm, 0.03 mm, 0.04 mm, 0.05 mm, 0.06 mm, 0.07 mm, 0.08 mm, 0.09 mm, 0.1 mm, 0.11 mm, 0.12 mm, 0.13 mm, 0.14 mm, 0.15 mm, 0.16 mm, 0.17 mm, 0.18 mm, 0.19 mm, 0.2 mm, 0.25 mm, 0.3 mm, 0.35 mm, 0.4 mm, 0.45 mm, 0.5 mm, 0.55 mm, 0.6 mm, 0.65 mm, 0.7 mm, 0.75 mm, 0.8 mm, 0.85 mm, 0.9 mm, 0.95 mm, 1 mm, 1.05 mm, 1.1 mm, 1.15 mm, 1.2 mm, 1.25 mm, 1.3 mm, 1.35 mm, 1.4 mm, 1.45 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.75 mm, 3 mm, 3.25 mm, 3.5 mm, 3.75 mm, 4 mm, 4.5 mm, or 5 mm. The particulate solids can be exogenously added to the hydrolyzate, saccharide stream, or saccharide solution. The particulate solids can be in the hydrolyzate, saccharide stream, or saccharide solution as a result of the biomass pretreatment and/or hydrolysis used to produce the hydrolyzate, saccharide stream, or saccharide solution.

Particulate solids (e.g., residual, insoluble or suspended solids) in a hydrolyzate, saccharide stream, or saccharide solution can have, for example, particle sizes of from about 1 μM³ to about 5 mm³ or larger or smaller. The particulate solids can have particles sizes of about 0.001-5 mm³, 0.01-2.5 mm³, 0.01-1 mm³, 0.01-0.5 mm³, 0.01-0.1 mm³, 0.01-0.05 mm³, 0.05-2.5 mm³, 0.05-1 mm³, 0.05-0.5 mm³, 0.05-0.1 mm³, 0.1-2.5 mm³, 0.1-1 mm³, 0.1-0.5 mm³, 0.5-2.5 mm³, 0.5-1 mm³, 1-2.5 mm³, 0.001 mm³, 0.002 mm³, 0.003 mm³, 0.004 mm³, 0.005 mm³, 0.006 mm³, 0.007 mm³, 0.008 mm³, 0.009 mm³, 0.01 mm³, 0.02 mm³, 0.03 mm³, 0.04 mm³, 0.05 mm³, 0.06 mm³, 0.07 mm³, 0.08 mm³, 0.09 mm³, 0.1 mm³, 0.11 mm³, 0.12 mm³, 0.13 mm³, 0.14 mm³, 0.15 mm³, 0.16 mm³, 0.17 mm³, 0.18 mm³, 0.19 mm³, 0.2 mm³, 0.25 mm³, 0.3 mm³, 0.35 mm³, 0.4 mm³, 0.45 mm³, 0.5 mm³, 0.55 mm³, 0.6 mm³, 0.65 mm³, 0.7 mm³, 0.75 mm³, 0.8 mm³, 0.85 mm³, 0.9 mm³, 0.95 mm³, 1 mm³, 1.05 mm³, 1.1 mm³, 1.15 mm³, 1.2 mm³, 1.25 mm³, 1.3 mm³, 1.35 mm³, 1.4 mm³, 1.45 mm³, 1.5 mm³, 1.6 mm³, 1.7 mm³, 1.8 mm³, 1.9 mm³, 2 mm³, 2.1 mm³, 2.2 mm³, 2.3 mm³, 2.4 mm³, 2.5 mm³, 2.75 mm³, 3 mm³, 3.25 mm³, 3.5 mm³, 3.75 mm³, 4 mm³, 4.5 mm³, or 5 mm³. The particulate solids can be exogenously added to the hydrolyzate, saccharide stream, or saccharide solution. The particulate solids can be in the hydrolyzate, saccharide stream, or saccharide solution as a result of the biomass pretreatment and/or hydrolysis used to produce the hydrolyzate, saccharide stream, or saccharide solution.

Particulate solids (e.g., residual, insoluble or suspended solids) in a hydrolyzate, saccharide stream, or saccharide solution can have, for example, average particle sizes of less than about 5 mm³. The particulate solids can have particles sizes of less than about 0.001 mm³, 0.002 mm³, 0.003 mm³, 0.004 mm³, 0.005 mm³, 0.006 mm³, 0.007 mm³, 0.008 mm³, 0.009 mm³, 0.01 mm³, 0.02 mm³, 0.03 mm³, 0.04 mm³, 0.05 mm³, 0.06 mm³, 0.07 mm³, 0.08 mm³, 0.09 mm³, 0.1 mm³, 0.11 mm³, 0.12 mm³, 0.13 mm³, 0.14 mm³, 0.15 mm³, 0.16 mm³, 0.17 mm³, 0.18 mm³, 0.19 mm³, 0.2 mm³, 0.25 mm³, 0.3 mm³, 0.35 mm³, 0.4 mm³, 0.45 mm³, 0.5 mm³, 0.55 mm³, 0.6 mm³, 0.65 mm³, 0.7 mm³, 0.75 mm³, 0.8 mm³, 0.85 mm³, 0.9 mm³, 0.95 mm³, 1 mm³, 1.05 mm³, 1.1 mm³, 1.15 mm³, 1.2 mm³, 1.25 mm³, 1.3 mm³, 1.35 mm³, 1.4 mm³, 1.45 mm³, 1.5 mm³, 1.6 mm³, 1.7 mm³, 1.8 mm³, 1.9 mm³, 2 mm³, 2.1 mm³, 2.2 mm³, 2.3 mm³, 2.4 mm³, 2.5 mm³, 2.75 mm³, 3 mm³, 3.25 mm³, 3.5 mm³, 3.75 mm³, 4 mm³, 4.5 mm³, or 5 mm³. The particulate solids can be exogenously added to the hydrolyzate, saccharide stream, or saccharide solution. The particulate solids can be in the hydrolyzate, saccharide stream, or saccharide solution as a result of the biomass pretreatment and/or hydrolysis used to produce the hydrolyzate, saccharide stream, or saccharide solution.

In some embodiments, all or a portion of the particulate solids (e.g., residual, insoluble or suspended solids) are sequestered and removed from a hydrolyzate, saccharide stream, or saccharide solution. The sequestration and removal can be accomplished, for example, by flocculation, filtration, evaporation, centrifugation, or a combination thereof. The removed particulate solids can be added to a fermentation reaction or saccharide solution as exogenous particulate solids. The addition of exogenous particulate solids can increase the production of polyols, fatty acids, and/or triacylglycerols in a fermentation reaction. The addition of exogenous particulate solids can increase, decrease, have substantially no effect, or have no effect upon the production of fermentation end-products such as alcohols (e.g., ethanol, methanol, propanol, butanol, etc.).

The level of particulate solids (e.g., residual, insoluble or suspended solids) in a hydrolyzate, saccharide stream, or saccharide solution can affect the rate and/or final titer of one or more fermentation end-products in a fermentation reaction. For example, increasing the level of residual solids can increase the rate of production and/or the final titer of fermentation end-products such as polyols, fatty acids, and/or triacylglycerols. Without being limited by theory, this can be due to increased osmotic stress upon the cells used in the fermentation reaction, increased irritation of the cells in the fermentation reaction, and/or increased nutrients or precursor molecules delivered to the cells in the fermentation reaction.

The level of particulate solids (e.g., residual, insoluble or suspended solids) in a hydrolyzate, saccharide stream, or saccharide solution can affect the growth rate of cells in a cell culture added to the hydrolyzate, saccharide stream, or saccharide solution. The particulate solids can contain nutrients (e.g., proteins, amino acids, fats, oils, etc.) or ions/trace metals that promote microorganism growth. Increased growth rates can increase, decrease, or have no effect upon the production of one or more fermentation end-products.

In one embodiment, pretreatment of biomass comprises ionic liquid (IL) pretreatment. Biomass can be pretreated by incubation with an ionic liquid, followed by IL extraction with a wash solvent such as alcohol or water. The treated biomass can then be separated from the ionic liquid/wash-solvent solution by centrifugation or filtration, and sent to the saccharification reactor or vessel. Examples of ionic liquid pretreatment are disclosed in US publication No. 2008/0227162, incorporated herein by reference in its entirety.

In another embodiment, a method can utilize a pretreatment process disclosed in U.S. Pat. No. 4,600,590 to Dale, U.S. Pat. No. 4,644,060 to Chou, U.S. Pat. No. 5,037,663 to Dale. U.S. Pat. No. 5,171,592 to Holtzapple, et al., et al., U.S. Pat. No. 5,939,544 to Karstens, et al., U.S. Pat. No. 5,473,061 to Bredereck, et al., U.S. Pat. No. 6,416,621 to Karstens, U.S. Pat. No. 6,106,888 to Dale, et al., U.S. Pat. No. 6,176,176 to Dale, et al., PCT publication WO2008/020901 to Dale, et al., Felix, A., et al., Anim. Prod. 51, 47-61 (1990), Wais, A. C., Jr., et al., Journal of Animal Science, 35, No. 1, 109-112 (1972), which are incorporated herein by reference in their entireties.

Alteration of the pH of a pretreated feedstock can be accomplished by washing the feedstock (e.g., with water) one or more times to remove an alkaline or acidic substance, or other substance used or produced during pretreatment. Washing can comprise exposing the pretreated feedstock to an equal volume of water 2, 3, 4, 5, 6, 7, 8, 9, 10, 11, 12, 13, 14, 15, 16, 17, 18, 19, 20, 21, 22, 23, 24, 25 or more times. In another embodiment, a pH modifier can be added. For example, an acid, a buffer, or a material that reacts with other materials present can be added to modulate the pH of the feedstock. In one embodiment, more than one pH modifier can be used, such as one or more bases, one or more bases with one or more buffers, one or more acids, one or more acids with one or more buffers, or one or more buffers. When more than one pH modifiers are utilized, they can be added at the same time or at different times. Other non-limiting exemplary methods for neutralizing feedstocks treated with alkaline substances have been described, for example in U.S. Pat. Nos. 4,048,341; 4,182,780; and 5,693,296.

In one embodiment, one or more acids can be combined, resulting in a buffer. Suitable acids and buffers that can be used as pH modifiers include any liquid or gaseous acid that is compatible with the microorganism. Non-limiting examples include peroxyacetic acid, sulfuric acid, lactic acid, citric acid, phosphoric acid, and hydrochloric acid. In some instances, the pH can be lowered to neutral pH or acidic pH, for example a pH of 7.0, 6.5, 6.0, 5.5, 5.0, 4.5, 4.0, or lower. In some embodiments, the pH is lowered and/or maintained within a range of about pH 4.5 to about 7.1, or about 4.5 to about 6.9, or about pH 5.0 to about 6.3, or about pH 5.5 to about 6.3, or about pH 6.0 to about 6.5, or about pH 5.5 to about 6.9 or about pH 6.2 to about 6.7.

In another embodiment, biomass can be pre-treated at an elevated temperature and/or pressure. In one embodiment biomass is pre treated at a temperature range of 20° C. to 400° C. In another embodiment biomass is pretreated at a temperature of about 20° C., 25° C., 30° C., 35° C., 40° C., 45° C., 50° C., 55° C., 60° C., 65° C., 80° C., 90° C., 100° C., 120° C., 150° C., 200° C., 250° C., 300° C., 350° C., 400° C. or higher. In another embodiment, elevated temperatures are provided by the use of steam, hot water, or hot gases. In one embodiment steam can be injected into a biomass containing vessel. In another embodiment the steam, hot water, or hot gas can be injected into a vessel jacket such that it heats, but does not directly contact the biomass.

In another embodiment, a biomass can be treated at an elevated pressure. In one embodiment biomass is pre treated at a pressure range of about 1 psi to about 30 psi. In another embodiment biomass is pre treated at a pressure or about 1 psi, 2 psi, 3 psi, 4 psi, 5 psi, 6 psi, 7 psi, 8 psi, 9 psi, 10 psi, 12 psi, 15 psi, 18 psi, 20 psi, 22 psi, 24 psi, 26 psi, 28 psi, 30 psi or more. In some embodiments, biomass can be treated with elevated pressures by the injection of steam into a biomass containing vessel. In one embodiment, the biomass can be treated to vacuum conditions prior or subsequent to alkaline or acid treatment or any other treatment methods provided herein.

In one embodiment alkaline or acid pretreated biomass is washed (e.g. with water (hot or cold) or other solvent such as alcohol (e.g. ethanol)), pH neutralized with an acid, base, or buffering agent (e.g. phosphate, citrate, borate, or carbonate salt) or dried prior to fermentation. In one embodiment, the drying step can be performed under vacuum to increase the rate of evaporation of water or other solvents. Alternatively, or additionally, the drying step can be performed at elevated temperatures such as about 20° C., 25° C., 30° C., 35° C., 40° C., 45° C., 50° C., 55° C., 60° C., 65° C., 80° C., 90° C., 100° C., 120° C., 150° C., 200° C., 250° C., 300° C. or more.

In one embodiment of the present invention, the pretreatment step includes a step of solids recovery. The solids recovery step can be during or after pretreatment (e.g., acid or alkali pretreatment), or before the drying step. In one embodiment, the solids recovery step provided by the methods of the present invention includes the use of a sieve, filter, screen, or a membrane for separating the liquid and solids fractions. In one embodiment a suitable sieve pore diameter size ranges from about 0.001 microns to 8 mm, such as about 0.005 microns to 3 mm or about 0.01 microns to 1 mm. In one embodiment a sieve pore size has a pore diameter of about 0.01 microns, 0.02 microns, 0.05 microns, 0.1 microns, 0.5 microns, 1 micron, 2 microns, 4 microns, 5 microns, 10 microns, 20 microns, 25 microns, 50 microns, 75 microns, 100 microns, 125 microns, 150 microns, 200 microns, 250 microns, 300 microns, 400 microns, 500 microns, 750 microns, 1 mm or more. In one embodiment, biomass (e.g. corn stover) is processed or pretreated prior to fermentation. In one embodiment a method of pre-treatment includes but is not limited to, biomass particle size reduction, such as for example shredding, milling, chipping, crushing, grinding, or pulverizing. In one embodiment, biomass particle size reduction can include size separation methods such as sieving, or other suitable methods known in the art to separate materials based on size. In one embodiment size separation can provide for enhanced yields. In one embodiment, separation of finely shredded biomass (e.g. particles smaller than about 8 mm in diameter, such as, 8, 7.9, 7.7, 7.5, 7.3, 7, 6.9, 6.7, 6.5, 6.3, 6, 5.9, 5.7, 5.5, 5.3, 5, 4.9, 4.7, 4.5, 4.3, 4, 3.9, 3.7, 3.5, 3.3, 3, 2.9, 2.7, 2.5, 2.3, 2, 1.9, 1.7, 1.5, 1.3, 1, 0.9, 0.8, 0.7, 0.6, 0.5, 0.4, 0.3, 0.2, or 0.1 mm) from larger particles allows the recycling of the larger particles back into the size reduction process, thereby increasing the final yield of processed biomass. In one embodiment, a fermentative mixture is provided which comprises a pretreated lignocellulosic feedstock comprising less than about 50% of a lignin component present in the feedstock prior to pretreatment and comprising more than about 60% of a hemicellulose component present in the feedstock prior to pretreatment; and a microorganism capable of fermenting a five-carbon sugar, such as xylose, arabinose or a combination thereof, and a six-carbon sugar, such as glucose, galactose, mannose or a combination thereof. In some instances, pretreatment of the lignocellulosic feedstock comprises adding an alkaline substance which raises the pH to an alkaline level, for example NaOH. In one embodiment, NaOH is added at a concentration of about 0.5% to about 2% by weight of the feedstock. In one embodiment, pretreatment also comprises addition of a chelating agent.

Hydrolysis

In one embodiment, the biomass hydrolyzing unit provides useful advantages for the conversion of biomass to biofuels and chemical products. One advantage of this unit is its ability to produce monomeric sugars from multiple types of biomass, including mixtures of different biomass materials, and is capable of hydrolyzing polysaccharides and higher molecular weight saccharides to lower molecular weight saccharides. In one embodiment, the hydrolyzing unit utilizes a pretreatment process and a hydrolytic enzyme which facilitates the production of a sugar stream containing a concentration of a monomeric sugar or several monomeric sugars derived from cellulosic and/or hemicellulosic polymers. Examples of biomass material that can be pretreated and hydrolyzed to manufacture sugar monomers include, but are not limited to, cellulosic, hemicellulosic, lignocellulosic materials; pectins; starches; wood; paper; agricultural products; forest waste; tree waste; tree bark; leaves; grasses; sawgrass; woody plant matter; non-woody plant matter; carbohydrates; starch; inulin; fructans; glucans; corn; sugar cane; sorghum, other grasses; bamboo, algae, and material derived from these materials. This ability to use a very wide range of pretreatment methods and hydrolytic enzymes gives distinct advantages in biomass fermentations. Various pretreatment conditions and enzyme hydrolysis can enhance the extraction of sugars from biomass, resulting in higher yields, higher productivity, greater product selectivity, and/or greater conversion efficiency.

In one embodiment, the enzyme treatment is used to hydrolyze various higher saccharides (higher molecular weight) present in biomass to lower saccharides (lower molecular weight), such as in preparation for fermentation by biocatalysts such as yeasts to produce ethanol, hydrogen, or other chemicals such as organic acids including succinic acid, formic acid, acetic acid, and lactic acid. These enzymes and/or the hydrolysate can be used in fermentations to produce various products including fuels, and other chemicals.

In one example, the process for converting biomass material into ethanol includes pretreating the biomass material (e.g., “feedstock”), hydrolyzing the pretreated biomass to convert polysaccharides to oligosaccharides, further hydrolyzing the oligosaccharides to monosaccharides, and converting the monosaccharides to biofuels and chemical products. Enzymes such as cellulases, polysaccharases, lipases, proteases, ligninases, and hemicellulases, help produce the monosaccharides can be used in the biosynthesis of fermentation end-products. Biomass material that can be utilized includes woody plant matter, non-woody plant matter, cellulosic material, lignocellulosic material, hemicellulosic material, carbohydrates, pectin, starch, inulin, fructans, glucans, corn, algae, sugarcane, other grasses, switchgrass, bagasse, wheat straw, barley straw, rice straw, corncobs, bamboo, citrus peels, sorghum, high biomass sorghum, seed hulls, and material derived from these. The final product can then be separated and/or purified, as indicated by the properties for the desired final product. In some instances, compounds related to sugars such as sugar alcohols or sugar acids can be utilized as well.

Chemicals used in the methods of the present invention are readily available and can be purchased from a commercial supplier, such as Sigma-Aldrich. Additionally, commercial enzyme cocktails (e.g. Accellerase™ 1000, CelluSeb-TL, CelluSeb-TS, Cellic™, CTec, STARGEN™, Maxalig™, Spezyme.R™, Distillase.R™, G-Zyme.R™, Fermenzyme.R™, Fermgen™, GC 212, or Optimash™) or any other commercial enzyme cocktail can be purchased from vendors such as Specialty Enzymes & Biochemicals Co., Genencor, or Novozymes. Alternatively, enzyme cocktails can be prepared by growing one or more organisms such as for example a fungi (e.g. a Trichoderma, a Saccharomyces, a Pichia, a White Rot Fungus etc.), a bacteria (e.g. a Clostridium, or a coliform bacterium, a Zymomonas bacterium, Sacharophagus degradans etc.) in a suitable medium and harvesting enzymes produced therefrom. In some embodiments, the harvesting can include one or more steps of purification of enzymes.

In one embodiment, treatment of biomass comprises enzyme hydrolysis. In one embodiment a biomass is treated with an enzyme or a mixture of enzymes, e.g., endoglucanases, exoglucanases, cellobiohydrolases, cellulase, beta-glucosidases, glycoside hydrolases, glycosyltransferases, lyases, esterases and proteins containing carbohydrate-binding modules. In one embodiment, the enzyme or mixture of enzymes is one or more individual enzymes with distinct activities. In another embodiment, the enzyme or mixture of enzymes can be enzyme domains with a particular catalytic activity. For example, an enzyme with multiple activities can have multiple enzyme domains, including for example glycoside hydrolases, glycosyltransferases, lyases and/or esterases catalytic domains.

In one embodiment, enzymes that degrade polysaccharides are used for the hydrolysis of biomass and can include enzymes that degrade cellulose, namely, cellulases. Examples of some cellulases include endocellulases and exo-cellulases that hydrolyze beta-1,4-glucosidic bonds.

In one embodiment, enzymes that degrade polysaccharides are used for the hydrolysis of biomass and can include enzymes that have the ability to degrade hemicellulose, namely, hemicellulases. Hemicellulose can be a major component of plant biomass and can contain a mixture of pentoses and hexoses, for example, D-xylopyranose, L-arabinofuranose, D-mannopyranose, Dglucopyranose, D-galactopyranose, D-glucopyranosyluronic acid and other sugars. In one embodiment, enzymes that degrade polysaccharides are used for the hydrolysis of biomass and can include enzymes that have the ability to degrade pectin, namely, pectinases. In plant cell walls, the cross-linked cellulose network can be embedded in a matrix of pectins that can be covalently cross-linked to xyloglucans and certain structural proteins. Pectin can comprise homogalacturonan (HG) or rhamnogalacturonan (RH).

In one embodiment, hydrolysis of biomass includes enzymes that can hydrolyze starch. Enzymes that hydrolyze starch include alpha-amylase, glucoamylase, beta-amylase, exo-alpha-1,4-glucanase, and pullulanase.

In one embodiment, hydrolysis of biomass comprises hydrolases that can include enzymes that hydrolyze chitin. In another embodiment, hydrolases can include enzymes that hydrolyze lichen, namely, lichenase.

In one embodiment, after pretreatment and/or hydrolysis by any of the above methods the feedstock contains cellulose, hemicellulose, soluble oligomers, simple sugars, lignin, volatiles and ash. The parameters of the hydrolysis can be changed to vary the concentration of the components of the pretreated feedstock. For example, in one embodiment a hydrolysis is chosen so that the concentration of soluble C5 saccharides is high and the concentration of lignin is low after hydrolysis. Examples of parameters of the hydrolysis include temperature, pressure, time, concentration, composition and pH.

In one embodiment, the parameters of the pretreatment and hydrolysis are changed to vary the concentration of the components of the pretreated feedstock such that concentration of the components in the pretreated and hydrolyzed feedstock is optimal for fermentation with a microbe such as a yeast or bacterium microbe.

In one embodiment, the parameters of the pretreatment are changed to encourage the release of the components of a genetically modified feedstock such as enzymes stored within a vacuole to increase or complement the enzymes synthesized by biocatalyst to produce optimal release of the fermentable components during hydrolysis and fermentation.

In one embodiment, the parameters of the pretreatment and hydrolysis are changed such that concentration of accessible cellulose in the pretreated feedstock is 1%, 5%, 10%, 12%, 13%, 14%, 15%, 16%, 17%, 19%, 20%, 30%, 40% or 50%. In one embodiment, the parameters of the pretreatment are changed such that concentration of accessible cellulose in the pretreated feedstock is 5% to 30%. In one embodiment, the parameters of the pretreatment are changed such that concentration of accessible cellulose in the pretreated feedstock is 10% to 20%.

In one embodiment, the parameters of the pretreatment are changed such that concentration of hemicellulose in the pretreated feedstock is 1%, 5%, 10%, 12%, 13%, 14%, 15%, 16%, 17%, 19%, 20%, 21%, 22%, 23%, 24%, 25%, 26%, 27%, 28%, 29%, 30%, 40% or 50%. In one embodiment, the parameters of the pretreatment are changed such that concentration of hemicellulose in the pretreated feedstock is 5% to 40%. In one embodiment, the parameters of the pretreatment are changed such that concentration of hemicellulose in the pretreated feedstock is 10% to 30%.

In one embodiment, the parameters of the pretreatment and hydrolysis are changed such that concentration of soluble oligomers in the pretreated feedstock is 1%, 10%, 15%, 20%, 25%, 30%, 35%, 40%, 45%, 50%, 55%, 60%, 65%, 70%, 75%, 80%, 85%, 90%, 95%, or 99%. Examples of soluble oligomers include, but are not limited to, cellobiose and xylobiose. In one embodiment, the parameters of the pretreatment are changed such that concentration of soluble oligomers in the pretreated feedstock is 30% to 90%. In one embodiment, the parameters of the pretreatment and/or hydrolysis are changed such that concentration of soluble oligomers in the pretreated feedstock is 45% to 80%.

In one embodiment, the parameters of the pretreatment and hydrolysis are changed such that concentration of simple sugars in the pretreated feedstock is 1%, 5%, 10%, 12%, 13%, 14%, 15%, 16%, 17%, 19%, 20%, 30%, 40% or 50%. In one embodiment, the parameters of the pretreatment and hydrolysis are changed such that concentration of simple sugars in the pretreated feedstock is 0% to 20%. In one embodiment, the parameters of the pretreatment and hydrolysis are changed such that concentration of simple sugars in the pretreated feedstock is 0% to 5%. Examples of simple sugars include, but are not limited to, C5 and C6 monomers and dimers.

In one embodiment, the parameters of the pretreatment are changed such that concentration of lignin in the pretreated and/or hydrolyzed feedstock is 1%, 5%, 10%, 12%, 13%, 14%, 15%, 16%, 17%, 19%, 20%, 30%, 40% or 50%. In one embodiment, the parameters of the pretreatment and/or hydrolysis are changed such that concentration of lignin in the pretreated feedstock is 0% to 20%. In one embodiment, the parameters of the pretreatment and/or hydrolysis are changed such that concentration of lignin in the pretreated feedstock is 0% to 5%. In one embodiment, the parameters of the pretreatment and hydrolysis are changed such that concentration of lignin in the pretreated and/or hydrolyzed feedstock is less than 1% to 2%. In one embodiment, the parameters of the pretreatment and/or hydrolysis are changed such that the concentration of phenolics is minimized.

In one embodiment, the parameters of the pretreatment and/or hydrolysis are changed such that concentration of furfural and low molecular weight lignin in the pretreated and/or hydrolyzed feedstock is less than 10%, 9%, 8%, 7%, 6%, 5%, 4%, 3%, 2%, or 1%. In one embodiment, the parameters of the pretreatment and/or hydrolysis are changed such that concentration of furfural and low molecular weight lignin in the pretreated and/or hydrolyzed feedstock is less than 1% to 2%.

In one embodiment, the parameters of the pretreatment and/or hydrolysis are changed such that the concentration of simple sugars is at least 75% to 85%, and the concentration of lignin is 0% to 5% and the concentration of furfural and low molecular weight lignin in the pretreated feedstock is less than 1% to 2%.

In one embodiment, the parameters of the pretreatment and/or hydrolysis are changed to obtain a high concentration of hemicellulose and a low concentration of lignin. In one embodiment, the parameters of the pretreatment and/or hydrolysis are changed to obtain a high concentration of hemicellulose and a low concentration of lignin such that concentration of the components in the pretreated stock is optimal for fermentation with a microbe such as biocatalyst.

In one embodiment, more than one of these steps can occur at any given time. For example, hydrolysis of the pretreated feedstock and hydrolysis of the oligosaccharides can occur simultaneously, and one or more of these can occur simultaneously to the conversion of monosaccharides to a fuel or chemical.

In another embodiment, an enzyme can directly convert the polysaccharide to monosaccharides. In some instances, an enzyme can hydrolyze the polysaccharide to oligosaccharides and the enzyme or another enzyme can hydrolyze the oligosaccharides to monosaccharides.

In another embodiment, the enzymes can be added to the fermentation or they can be produced by microorganisms present in the fermentation. In one embodiment, the microorganism present in the fermentation produces some enzymes. In another embodiment, enzymes are produced separately and added to the fermentation.

For the overall conversion of pretreated biomass to final product to occur at high rates, it is generally necessary for each of the necessary enzymes for each conversion step to be present with sufficiently high activity. If one of these enzymes is missing or is present in insufficient quantities, the production rate of an end product will be reduced. The production rate can also be reduced if the microorganisms responsible for the conversion of monosaccharides to product only slowly take up monosaccharides and/or have only limited capability for translocation of the monosaccharides and intermediates produced during the conversion to end product. Additions of fractions obtained from pretreatment and/or pretreatment and hydrolysis can increase initial or overall growth rates. In another embodiment, oligomers are taken up slowly by a biocatalyst, necessitating an almost complete conversion of polysaccharides and oligomers to monomeric sugars.

In another embodiment, the enzymes of the method are produced by a biocatalyst, including a range of hydrolytic enzymes suitable for the biomass materials used in the fermentation methods. In one embodiment, a biocatalyst is grown under conditions appropriate to induce and/or promote production of the enzymes needed for the saccharification of the polysaccharide present. The production of these enzymes can occur in a separate vessel, such as a seed fermentation vessel or other fermentation vessel, or in the production fermentation vessel where ethanol production occurs. When the enzymes are produced in a separate vessel, they can, for example, be transferred to the production fermentation vessel along with the cells, or as a relatively cell free solution liquid containing the intercellular medium with the enzymes. When the enzymes are produced in a separate vessel, they can also be dried and/or purified prior to adding them to the hydrolysis or the production fermentation vessel. The conditions appropriate for production of the enzymes are frequently managed by growing the cells in a medium that includes the biomass that the cells will be expected to hydrolyze in subsequent fermentation steps. Additional medium components, such as salt supplements, growth factors, and cofactors including, but not limited to phytate, amino acids, and peptides can also assist in the production of the enzymes utilized by the microorganism in the production of the desired products.

### Fermentation

Provided herein are methods and systems to increase the yield of fermentation end-products such as polyols, fatty acids, and/or triacylglycerols. Such methods can involve fermentation of saccharide solutions produced from the pretreatment and hydrolysis of biomass compositions containing cellulose, hemicellulose, and/or lignocellulose. The saccharide solutions can contain C5 monosaccharides and/or C6 monosaccharides. The saccharide solution can also contain particulate solids and/or one or more osmotic agents. The particulate solids and/or osmotic agents can be exogenously added. The particulate solids and/or osmotic agents can be caused by or produced during the pretreatment and/or hydrolysis of the biomass composition. Higher levels of particulate solids and/or osmotic agents can cause the increased yields of the polyols, fatty acids, and/or triacylglycerols.

Exposing cells (e.g., plant cells, bacterial cells, yeast cells, algal cells, etc.) to a hypertonic solution can cause an efflux of cellular water into the medium. In order to counteract the outflow of water molecules during growth, cells can produce and accumulate one or more osmoregulatory molecules such as polyhydroxy compounds. (e.g., see Nevoit and Stahl (1997) FEMS Microbiology Review 21:231-241 and Parekh and Pandey (1985) Biotechnology and Bioengineering 27: 1089-1091, each of which is incorporated by reference in its entirety). Cells can direct part of the carbon substrate (e.g., C5 and/or C6 monosaccharides) to one or more fermentation end-products such as polyols, fatty acids, and/or triacylglycerols. In cells capable of their production, this can decrease the yield of fermentation end-products such as alcohols like ethanol. This can occur, for example, when the level of saccharides and/or other osmotic agents (e.g., salts, minerals, etc.) are overly abundant during the fermentation reaction. Environmental factors affecting these pathways can include oxygen availability, type of nitrogen source, osmotic pressure, heat and pH. For example, when glucose is overly abundant, a high osmotic pressure can shift metabolism to the production of glycerol.

Without being limited by theory, the high level of particulate solids (e.g., residual solids, insoluble solids or suspended solids) in a fermentation reaction or cell culture can cause osmotic stress upon the cells (e.g., plant cells, yeast cells, bacteria cells, algal cells, etc.). The osmotic stress can cause the microorganisms to produce osmoregulatory compounds such as polyols (e.g., glycerol). The cells can also produce higher levels of fatty acids and/or triglycerides. The particulate solids can contain nutrients and/or precursor molecules that also increase the production of these fermentation end-products. The particulate solids can also cause cell irritation, also increasing the production of these fermentation end-products.

In one aspect, disclosed are methods of producing one or more fermentation end-products comprising contacting a cell culture with a saccharide solution comprising C5 monosaccharides and/or C6 monosaccharides and particulate solids; and allowing sufficient time for cells in the cell culture to produce one or more fermentation end-products comprising one or more polyols, fatty acids, triacylglycerols, or a combination thereof. Using such methods, a greater yield of the one or more polyols, fatty acids, or triacylglycerols can be produced in comparison to fermentation of a saccharide solution comprising a lower level of the particulate solids. In some embodiments, the saccharide solution further comprises one or more osmotic agents. Some embodiments further comprise adding one or more exogenous osmotic agents to the saccharide solution. Some embodiments further comprise adding exogenous particulate solids to the saccharide solution.

In another aspect, disclosed are methods of producing one or more fermentation end-products comprising: adding exogenous particulate solids to a saccharide solution comprising C5 monosaccharides and/or C6 monosaccharides; contacting the saccharide solution with a cell culture; and allowing sufficient time for cells in the cell culture to produce one or more fermentation end-products comprising one or more polyols, fatty acids, triacylglycerols, or a combination thereof. Using such methods, a greater yield of the one or more polyols, fatty acids, or triacylglycerols can be produced in comparison to fermentation of a saccharide solution without the exogenous particulate solids. In some embodiments, the saccharide solution further comprises particulate solids. In some embodiments, the saccharide solution further comprises one or more osmotic agents. Some embodiments further comprise adding one or more exogenous osmotic agents to the saccharide solution.

In a further aspect, disclosed are methods of producing one or more fermentation end-products comprising adding one or more exogenous osmotic agents to a saccharide solution comprising C5 monosaccharides and/or C6 monosaccharides; contacting the saccharide solution with a cell culture; and allowing sufficient time for cells in the cell culture to produce one or more fermentation end-products comprising one or more polyols, fatty acids, triacylglycerols, or a combination thereof. Using such methods, a greater yield of the one or more polyols, fatty acids, or triacylglycerols can be produced in comparison to fermentation of a saccharide solution without the one or more exogenous osmotic agents. In some embodiments, the saccharide solution further comprises one or more osmotic agents. In some embodiments, the saccharide solution further comprises particulate solids. Some embodiments further comprise adding exogenous particulate solids to the saccharide solution.

The one or more polyols produced using the methods disclosed herein can include glycol, glycerol, erythritol, threitol, arabitol, xylitol, ribitol, mannitol, sorbitol, dulcitol, fucitol, iditol, inositol, volemitol, isomalt, maltitol, lactitol, polyglycitol. In one embodiment, the one or more polyols comprise glycerol.

The one or more fatty acids produced using the methods disclosed herein can include butyric acid, hexanoic acid, octanoic acid, decanoic acid, lauric acid, tridecanoic acid, myristic acid, pentadecanoic acid, palmitic acid, heptadecanoic acid, stearic acid, arachidic acid, heneicosanoic acid, behenic acid, tricosanoic acid, lignoceric acid, (cis-9) myristoleic acid, (cis-10) pentadecinoic acid, (cis-9) palmitoleic acid, (cis-10) heptadecenoate acid, (cis-9) oleic acid, (cis-11) eicosenoic acid, (cis-13) erucic acid, (cis-15) nervonic acid, (cis-9, 12) lonoleic acid, (cis-6, 9, 12) y-linolenic acid, (cis-9, 12,15) linolenic acid, (cis-11, 14) eicosadienoic acid, (cis-8, 11,14) eicosatrienoic acid, (cis-11, 14, 17) eicosatrienoic acid, (cis-5, 8, 11, 14) arachidonic acid, (cis-5, 8, 11, 14, 17) eicosapentanoic acid, (cis-13, 16) docosadienoic acid, (cis-4, 7, 10, 13, 16, 19) docosahexaenoic acid, (trans-9) methyl elaidate acid, (trans-9, 12) methyl linoelaidate acid, or a combination thereof. Triacylglycerols produced using the methods disclosed herein can comprise any of these fatty acids.

In some embodiments, the triacylglycerols produced using the methods disclosed herein are substantially the same as commercially available oils. For example, the triacylglycerols produced can be substantially the same as castor oil, coconut oil, colza oil, corn oil, cottonseed oil, false flax oil, hemp oil, mustard oil, palm oil, canola oil, peanut oil, radish oil, rapeseed oil, ramtil oil, rice bran oil, safflower oil, salicornia oil, soybean oil, sunflower oil, tigernut oil, tung oil, capaiba oil, honge oil, jatropha oil, jojoba oil, milk bush, nahor oil, paradise oil, or petroleum nut oil. Such oils can be used in the production of biodiesel.

The cell culture used to produce the fermentation end-products (e.g., polyols, fatty acids, triacylglycerols) can include plant cells, bacterial cells, yeast cells, algal cells, or a combination thereof. The plant cells can be, for example, from any of the following species: Ricinus communis, Cocos nucifera, Brassica rapa, var. oleifera, Zea mays, Gossypium hirsutum, Gossypium herbaceum, Camelina sativa, Cannabis sativa, Brassica nigra, Brassica juncea, Brassica hirta, Elaeis guineensis, Elaeis oleifera, Attalea maripa, Arachis hypogaea, Raphanus sativus, Brassica napus, Guizotia oleifera, Guizotia abyssinica, Oryza sativa, Oryza glaberrima, Carthamus tinctorius L., Salicornia bigelovii, Glycine max, Helianthus annuus, Cyperus esculentus, Vernicia fordii, a Copaifera species, Millettia pinnata, Jatropha curcas, Simmondsia chinensis, Euphorbia tirucalli, Mesua ferrea, Simarouba glauca, Pittosporum resiniferum, or any other plant species. The bacterial cells can be from a gram⁺ or gram⁻ species. The bacterial cells can be, for example, from a Rhodococcus strain, a Clostridium strain, a Trichoderma strain, a Saccharomyces strain, a Zymomonas strain, or a combination thereof. In one embodiment, the bacterial cells are from a Rhodococcus opacus strain. The cells in the cell culture can be genetically modified. The cells in the cell culture can be unmodified.

The increased yield of the polyols, fatty acids and/or triacylglycerols can be from about 1% to about 500% higher. For example, the increased yield can be about 1-500%, 1-300%, 1-200%, 1-150%, 1-100%, 1-75%, 1-50%, 1-25%, 1-10%, 1-5%, 5-500%, 5-300%, 5-200%, 5-150%, 5-100%, 5-75%, 5-50%, 5-25%, 5-10%, 10-500%, 10-300%, 10-200%, 10-150%, 10-100%, 10-75%, 10-50%, 10-25%, 25-500%, 25-300%, 25-200%, 25-150%, 25-100%, 25-75%, 25-50%, 50-500%, 50-300%, 50-200%, 50-150%, 50-100%, 50-75%, 75-500%, 75-300%, 75-200%, 75-150%, 75-100%, 100-500%, 100-300%, 100-200%, 100-150%, 150-500%, 150-300%, 150-200%, 200-500%, 200-300%, or 300-500% higher.

The increased yield of the polyols, fatty acids and/or triacylglycerols can be at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 27.5%, 30%, 32.5%, 35%, 37.5%, 40%, 42.5%, 45%, 47.5%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 275%, 300%, 325%, 350%, 375%, 400%, 425%, 450%, 475%, or 500% higher.

The saccharide solution can contain from about 0.001% to about 50% particulate solids w/v. For example, the saccharide solution can contain about 0.001-50%, 0.01-50%, 0.1-50%, 0.001-25%, 0.01-25%, 0.1-25%, 0.001-10%, 0.01-10%, 0.1-10%, 0.001-5%, 0.01-5%, 0.1-5%, 1-5%, 1-50%, 10-50%, 20-40%, 20-36%, 20-35%, 20-34%, 20-33%, 20-32%, 20-31%, 20-30%, 25-36%, 25-35%, 25-34%, 25-33%, 25-32%, 25-31%, 25-30%, 30-36%, 30-35%, 30-34%, 30-33%, 30-32%, or 30-31% particulate solids w/v. The particulate solids can be exogenously added to the saccharide solution. In some embodiments, a growth rate of cells in the cell culture is faster in saccharide solutions with the exogenous particulate solids than in saccharide solutions without the exogenous particulate solids. The particulate solids can be in the saccharide solution as a result of the biomass pretreatment and/or hydrolysis used to produce the hydrolyzate, saccharide stream, or saccharide solution. The particulate solids can comprise cellulosic particles, hemicellulosic particles, lignin particles, or a combination thereof. In some embodiments, a growth rate of cells in the cell culture is faster in saccharide solutions higher levels of particulate solids than in saccharide solutions that are otherwise the same.

The saccharide solution can contain one or more osmotic agents such as one or more salts, acid-solubilized lignin, one or more fatty acids, one or more metal ions, one or more trace elements, one or more acids, one or more bases, ash, one or more organic acids, one or more alcohols, or a combination thereof. The one or more osmotic agents can be exogenously added to the saccharide solution. The one or more osmotic agents can be in the saccharide solution as a result of a pretreatment and/or hydrolysis process. The one or more osmotic agents can comprise salts that were formed by the neutralization of a base or an acid.

The one or more osmotic agents can be one or more metal ions such as aluminum, antimony, arsenic, barium, cadmium, calcium, chromium, cobalt, copper, iron, lead, magnesium, manganese, nickel, phosphorus, potassium, selenium, silver, sodium, tin, vanadium, zinc. Any of the one or more metal ions can be present in the saccharide solution at a level, or added to the saccharide solution to the level that is from about 0.01 to about 5000 PPM (particles per million). For example, any of the metal ions can be in the saccharide solution, with or without exogenous addition, at about 0.1-3000 PPM, 0.1-2000 PPM, 0.1-1500 PPM, 0.1-1000 PPM, 0.1-750 PPM, 0.1-500 PPM, 0.1-250 PPM, 0.1-100 PPM, 0.1-50 PPM, 0.1-10 PPM, 0.1-5 PPM, 0.1-1 PPM, 1-3000 PPM, 1-2000 PPM, 1-1500 PPM, 1-1000 PPM, 1-750 PPM, 1-500 PPM, 1-250 PPM, 1-100 PPM, 1-50 PPM, 1-10 PPM, 1-5 PPM, 5-3000 PPM, 5-2000 PPM, 5-1500 PPM, 5-1000 PPM, 5-750 PPM, 5-500 PPM, 5-250 PPM, 5-100 PPM, 5-50 PPM, 5-10 PPM, 10-3000 PPM, 10-2000 PPM, 10-1500 PPM, 10-1000 PPM, 10-750 PPM, 10-500 PPM, 10-250 PPM, 10-100 PPM, 10-50 PPM, 50-3000 PPM, 50-2000 PPM, 50-1500 PPM, 50-1000 PPM, 50-750 PPM, 50-500 PPM, 50-250 PPM, 50-100 PPM, 100-3000 PPM, 100-2000 PPM, 100-1500 PPM, 100-1000 PPM, 100-750 PPM, 100-500 PPM, 100-250 PPM, 250-3000 PPM, 250-2000 PPM, 250-1500 PPM, 250-1000 PPM, 250-750 PPM, 250-500 PPM, 500-3000 PPM, 500-2000 PPM, 500-1500 PPM, 500-1000 PPM, 500-750 PPM, 750-3000 PPM, 750-2000 PPM, 750-1500 PPM, 750-1000 PPM, 1000-3000 PPM, 1000-2000 PPM, 1000-1500 PPM, 1500-3000 PPM, 1500-2000 PPM, or 2000-3000 PPM.

The osmolarity of the saccharide solution, with or without addition of exogenous osmotic agents, can be from about 125 mOsm/L to about 3500 mOsm/L. For example, the osmolarity of the saccharide solution can be about 125-3500 mOsm/L, 125-3000 mOsm/L, 125-2500 mOsm/L, 125-2000 mOsm/L, 125-1750 mOsm/L, 125-1500 mOsm/L, 125-1250 mOsm/L, 125-1000 mOsm/L, 125-750 mOsm/L, 125-500 mOsm/L, 125-250 mOsm/L, 250-3500 mOsm/L, 250-3000 mOsm/L, 250-2500 mOsm/L, 250-2000 mOsm/L, 250-1750 mOsm/L, 250-1500 mOsm/L, 250-1250 mOsm/L, 250-1000 mOsm/L, 250-750 mOsm/L, 250-500 mOsm/L, 500-3500 mOsm/L, 500-3000 mOsm/L, 500-2500 mOsm/L, 500-2000 mOsm/L, 500-1750 mOsm/L, 500-1500 mOsm/L, 500-1250 mOsm/L, 500-1000 mOsm/L, 500-750 mOsm/L, 750-3500 mOsm/L, 750-3000 mOsm/L, 750-2500 mOsm/L, 750-2000 mOsm/L, 750-1750 mOsm/L, 750-1500 mOsm/L, 750-1250 mOsm/L, 750-1000 mOsm/L, 1000-3500 mOsm/L, 1000-3000 mOsm/L, 1000-2500 mOsm/L, 1000-2000 mOsm/L, 1000-1750 mOsm/L, 1000-1500 mOsm/L, 1000-1250 mOsm/L, 1250-3500 mOsm/L, 1250-3000 mOsm/L, 1250-2500 mOsm/L, 1250-2000 mOsm/L, 1250-1750 mOsm/L, 1250-1500 mOsm/L, 1500-3500 mOsm/L, 1500-3000 mOsm/L, 1500-2500 mOsm/L, 1500-2000 mOsm/L, 1500-1750 mOsm/L, 1750-3500 mOsm/L, 1750-3000 mOsm/L, 1750-2500 mOsm/L, 1750-2000 mOsm/L, 2000-3500 mOsm/L, 2000-3000 mOsm/L, 2000-2500 mOsm/L, 2500-3500 mOsm/L, 2500-3000 mOsm/L, or 3000-3500 mOsm/L. The osmolarity of the saccharide solution can be due to C5 saccharides, C6 saccharides, and/or one or more osmotic agents. The one or more osmotic agents can be exogenously added.

The osmolarity of the saccharide solution, with or without addition of exogenous osmotic agents can be about 125 mOsm/L, 150 mOsm/L, 175 mOsm/L, 200 mOsm/L, 225 mOsm/L, 250 mOsm/L, 275 mOsm/L, 300 mOsm/L, 325 mOsm/L, 350 mOsm/L, 375 mOsm/L, 400 mOsm/L, 425 mOsm/L, 450 mOsm/L, 475 mOsm/L, 500 mOsm/L, 550 mOsm/L, 600 mOsm/L, 650 mOsm/L, 700 mOsm/L, 750 mOsm/L, 800 mOsm/L, 850 mOsm/L, 900 mOsm/L, 950 mOsm/L, 1000 mOsm/L, 1100 mOsm/L, 1200 mOsm/L, 1300 mOsm/L, 1400 mOsm/L, 1500 mOsm/L, 1600 mOsm/L, 1700 mOsm/L, 1800 mOsm/L, 1900 mOsm/L, 2000 mOsm/L, 2100 mOsm/L, 2200 mOsm/L, 2300 mOsm/L, 2400 mOsm/L, 2500 mOsm/L, 2600 mOsm/L, 2700 mOsm/L, 2800 mOsm/L, 2900 mOsm/L, 3000 mOsm/L, 3100 mOsm/L, 3200 mOsm/L, 3300 mOsm/L, 3400 mOsm/L, or 3500 mOsm/L.

Exogenous osmotic agents can be added to a level that increases the osmolarity of the saccharide solution by from about 0.01% to about 50%. For example, the level of exogenous osmotic agents can increase the osmolarity of the saccharide solution by about 0.01-50%, 0.01-35%, 0.01-25%, 0.01-20%, 0.01-15%, 0.01-10%, 0.01-5%, 0.01-1%, 0.01-0.1%, 0.1-50%, 0.1-35%, 0.1-25%, 0.1-20%, 0.1-15%, 0.1-10%, 0.1-5%, 0.1-1%, 1-50%, 1-35%, 1-25%, 1-20%, 1-15%, 1-10%, 1-5%, 5-50%, 5-35%, 5-25%, 5-20%, 5-15%, 5-10%, 10-50%, 10-35%, 10-25%, 10-20%, 10-15%, 15-50%, 15-35%, 15-25%, 15-20%, 20-50%, 20-35%, 20-25%, 25-50%, 25-35%, or 35-50%.

In some embodiments, a saccharide solution comprising C5 and/or C6 monosaccharides is produced by pretreating and/or hydrolyzing a biomass composition comprising cellulosic, hemicellulosic, and/or lignocellulosic material. The biomass composition can comprise corn, corn syrup, corn stover, corn cobs, molasses, silage, grass, straw, grain hulls, bagasse, distiller's grains, distiller's dried solubles, distiller's dried grains, condensed distiller's solubles, distiller's wet grains, distiller's dried grains with solubles, wood, bark, sawdust, paper, poplars, willows, switchgrass, alfalfa, prairie bluestem, algae, fruit peels, pits, sorghum, sweet sorghum, sugar cane, switch grass, rice, rice straw, rice hulls, wheat, wheat straw, barley, barley straw, bamboo, seeds, seed hulls, oats, oat hulls, food waste, municipal sewage waste, or a combination thereof. In some embodiments, pretreatment and/or hydrolysis of the biomass composition comprises mechanical size reduction, treatment with one or more acids, treatment with one or more bases, treatment with one or more enzymes, thermal treatment, stream explosion, acid-catalyzed steam explosion, ammonia fiber explosion, or a combination thereof.

The present disclosure also provides a fermentative mixture comprising: a cellulosic feedstock pre-treated with an alkaline or acid substance and at a temperature of from about 80° C. to about 120° C.; subsequently hydrolyzed with an enzyme mixture, and a microorganism capable of fermenting a five-carbon sugar and/or a six-carbon sugar. In one embodiment, the five-carbon sugar is xylose, arabinose, or a combination thereof. In one embodiment, the six-carbon sugar is glucose, galactose, mannose, or a combination thereof. In one embodiment, the alkaline substance is NaOH. In some embodiments, NaOH is added at a concentration of about 0.5% to about 2% by weight of the feedstock. In one embodiment, the acid is equal to or less than 2% HCl or H₂SO₄. In one embodiment, the microorganism is a Rhodococcus strain, a Clostridium strain, a Trichoderma strain, a Saccharomyces strain, a Zymomonas strain, or another microorganism suitable for fermentation of biomass. In another embodiment, the fermentation process comprises fermentation of the biomass using a microorganism that is Clostridium phytofermentans, Clostridium algidixylanolyticum, Clostridium xylanolyticum, Clostridium cellulovorans, Clostridium cellulolyticum, Clostridium thermocellum, Clostridium josui, Clostridium papyrosolvens, Clostridium cellobioparum, Clostridium hungatei, Clostridium cellulosi, Clostridium stercorarium, Clostridium termitidis, Clostridium thermocopriae, Clostridium celerecrescens, Clostridium polysaccharolyticum, Clostridium populeti, Clostridium lentocellum, Clostridium chartatabidum, Clostridium aldrichii, Clostridium herbivorans, Acetivibrio cellulolyticus, Bacteroides cellulosolvens, Caldicellulosiruptor saccharolyticum, Rhodococcus opacus, Ruminococcus albus, Ruminococcus flavefaciens, Fibrobacter succinogenes, Eubacterium cellulosolvens, Butyrivibrio fibrisolvens, Anaerocellum thermophilum, Halocella cellulolytica, Thermoanaerobacterium thermosaccharolyticum, Sacharophagus degradans, or Thermoanaerobacterium saccharolyticum. In still another embodiment, the microorganism is genetically modified to enhance activity of one or more hydrolytic enzymes, such as a genetically-modified Saccaromyces cerevisae.

In one embodiment a wild type or a genetically-improved microorganism can be used for chemical production by fermentation. Methods to produce a genetically-improved strain can include genetic modification, mutagenesis, and adaptive processes, such as directed evolution. For example, yeasts can be genetically-modified to ferment C5 sugars. Other useful yeasts are species of Candida, Cryptococcus, Debaryomyces, Deddera, Hanseniaspora, Kluyveromyces, Pichia, Schizosaccharomyces, and Zygosaccharomyces. Rhodococus strains, such as Rhodococcus opacus variants are a source of triacylglycerols and other storage lipids. (See, e.g., Waltermann, et al., Microbiology 146:1143-1149 (2000)). Other useful organisms for fermentation include, but are not limited to, yeasts, especially Saccaromyces strains and bacteria such as Clostridium phytofermentans, Thermoanaerobacter ethanolicus, Clostridium thermocellum, Clostridium beijerinickii, Clostridium acetobutylicum, Clostridium tyrobutyricum, Clostridium thermobutyricum, Thermoanaerobacterium saccharolyticum, Thermoanaerobacter thermohydrosulfuricus, Clostridium acetobutylicum, Moorella ssp., Carboxydocella ssp., Zymomonas mobilis, recombinant E. coli, Klebsiella oxytoca, Rhodococcus opacus and Clostridium beijerickii.

An advantage of yeasts are their ability to grow under conditions that include elevated ethanol concentration, high sugar concentration, low sugar concentration, and/or operate under anaerobic conditions. These characteristics, in various combinations, can be used to achieve operation with long or short fermentation cycles and can be used in combination with batch fermentations, fed batch fermentations, self-seeding/partial harvest fermentations, and recycle of cells from the final fermentation as inoculum.

Examples of yeasts that can be used as a biocatalyst or fermentive microorganism in the methods disclosed herein include but are not limited to, species found in the genus Ascoidea, Brettanomyces, Candida, Cephaloascus, Coccidiascus, Dipodascus, Eremothecium, Galactomyces, Kluyveromyces, Pichia, Saccharomyces, Schizosaccharomyces, Sporopachydermia, Torulaspora, Yarrowia, or Zygosaccharomyces; for example, Ascoidea rebescens, Brettanomyces anomalus, Brettanomyces bruxellensis, Brettanomyces claussenii, Brettanomyces custersianus, Brettanomyces lambicus, Brettanomyces naardenensis, Brettanomyces nanus, Candida albicans, Candida ascalaphidarum, Candida amphixiae, Candida antarctica, Candida argentea, Candida atlantica, Candida atmosphaerica, Candida blattae, Candida carpophila, Candida cerambycidarum, Candida chauliodes, Candida corydali, Candida dosseyi, Candida dubliniensis, Candida ergatensis, Candida fructus, Candida glabrata, Candida fermentati, Candida guilliermondii, Candida haemulonii, Candida insectamens, Candida insectorum, Candida intermedia, Candida jeffresii, Candida kefyr, Candida krusei, Candida lusitaniae, Candida lyxosophila, Candida maltosa, Candida marina, Candida membranifaciens, Candida milleri, Candida oleophila, Candida oregonensis, Candida parapsilosis, Candida quercitrusa, Candida rugosa, Candida sake, Candida shehatea, Candida temnochilae, Candida tenuis, Candida tropicalis, Candida tsuchiyae, Candida sinolaborantium, Candida sojae, Candida subhashii, Candida viswanathii, Candida utilis, Cephaloascus fragrans, Coccidiascus legeri, Dypodascus albidus, Eremothecium cymbalariae, Galactomyces candidum, Galactomyces geotrichum, Kluyveromyces aestuarii, Kluyveromyces africanus, Kluyveromyces bacillisporus, Kluyveromyces blattae, Kluyveromyces dobzhanskii, Kluyveromyces hubeiensis, Kluyveromyces lactis, Kluyveromyces lodderae, Kluyveromyces marxianus, Kluyveromyces nonfermentans, Kluyveromyces piceae, Kluyveromyces sinensis, Kluyveromyces thermotolerans, Kluyveromyces waltii, Kluyveromyces wickerhamii, Kluyveromyces yarrowii, Pichia anomola, Pichia heedii, Pichia guilliermondii, Pichia kluyveri, Pichia membranifaciens, Pichia norvegensis, Pichia ohmeri, Pichia pastoris, Pichia subpelliculosa, Saccharomyces bayanus, Saccharomyces boulardii, Saccharomyces bulderi, Saccharomyces cariocanus, Saccharomyces cariocus, Saccharomyces cerevisiae, Saccharomyces chevalieri, Saccharomyces dairenensis, Saccharomyces ellipsoideus, Saccharomyces eubayanus, Saccharomyces exiguus, Saccharomyces florentinus, Saccharomyces kluyveri, Saccharomyces martiniae, Saccharomyces monacensis, Saccharomyces norbensis, Saccharomyces paradoxus, Saccharomyces pastorianus, Saccharomyces spencerorum, Saccharomyces turicensis, Saccharomyces unisporus, Saccharomyces uvarum, Saccharomyces zonatus, Schizosaccharomyces cryophilus, Schizosaccharomyces japonicus, Schizosaccharomyces octosporus, Schizosaccharomyces pombe, Sporopachydermia cereana, Sporopachydermia lactativora, Sporopachydermia quercuum, Torulaspora delbrueckii, Torulaspora franciscae, Torulaspora globosa, Torulaspora pretoriensis, Yarrowia lipolytica, Zygosaccharomyces bailii, Zygosaccharomyces bisporus, Zygosaccharomyces cidri, Zygosaccharomyces fermentati, Zygosaccharomyces florentinus, Zygosaccharomyces kombuchaensis, Zygosaccharomyces lentus, Zygosaccharomyces mellis, Zygosaccharomyces microellipsoides, Zygosaccharomyces mrakii, Zygosaccharomyces pseudorouxii, or Zygosaccharomyces rouxii, or a variant or genetically modified version thereof.

Examples of bacteria that can be used as a biocatalyst or fermentive microorganism in the methods disclosed herein include but are not limited to any bacterium found in the genus of Butyrivibrio, Ruminococcus, Eubacterium, Bacteroides, Acetivibrio, Caldibacillus, Acidothermus, Cellulomonas, Curtobacterium, Micromonospora, Actinoplanes, Streptomyces, Thermobifida, Thermomonospora, Microbispora, Fibrobacter, Sporocytophaga, Cytophaga, Flavobacterium, Achromobacter, Xanthomonas, Cellvibrio, Pseudomonas, Myxobacter, Escherichia, Klebsiella, Thermoanaerobacterium, Thermoanaerobacter, Geobacillus, Saccharococcus, Paenibacillus, Bacillus, Caldicellulosiruptor, Anaerocellum, Anoxybacillus, Zymomonas, Clostridium; for example, Butyrivibrio fibrisolvens, Ruminococcus flavefaciens, Ruminococcus succinogenes, Ruminococcus albus, Eubacterium cellulolyticum, Bacteroides cellulosolvens, Acetivibrio cellulolyticus, Acetivibrio cellulosolvens, Caldibacillus cellulovorans, Bacillus circulans, Acidothermus cellulolyticus, Cellulomonas cartae, Cellulomonas cellasea, Cellulomonas cellulans, Cellulomonas fimi, Cellulomonas flavigena, Cellulomonas gelida, Cellulomonas iranensis, Cellulomonas persica, Cellulomonas uda, Curtobacterium falcumfaciens, Micromonospora melonosporea, Actinoplanes aurantiaca, Streptomyces reticuli, Streptomyces alboguseolus, Streptomyces aureofaciens, Streptomyces cellulolyticus, Streptomyces flavogriseus, Streptomyces lividans, Streptomyces nitrosporeus, Streptomyces olivochromogenes, Streptomyces rochei, Streptomyces thermovulgaris, Streptomyces viridosporus, Thermobifida alba, Thermobifida fusca, Thermobifida cellulolytica, Thermomonospora curvata, Microbispora bispora, Fibrobacter succinogenes, Sporocytophaga myxococcoides, Cytophaga sp., Flavobacterium johnsoniae, Achromobacter piechaudii, Xanthomonas sp., Cellvibrio vulgaris, Cellvibrio fulvus, Cellvibrio gilvus, Cellvibrio mixtus, Pseudomonas fluorescens, Pseudomonas mendocina, Myxobacter sp. AL-1, Escherichia albertii, Escherichia blattae, Escherichia coli, Escherichia fergusonii, Escherichia hermannii, Escherichia vulneris, Klebsiella granulomatis, Klebsiella oxytoca, Klebsiella pneumonia, Klebsiella terrigena, Thermoanaerobacterium thermosulfurigenes, Thermoanaerobacterium aotearoense, Thermoanaerobacterium polysaccharolyticum, Thermoanaerobacterium zeae, Thermoanaerobacterium xylanolyticum, Thermoanaerobacterium saccharolyticum, Thermoanaerobium brockii, Thermoanaerobacterium thermosaccharolyticum, Thermoanaerobacter thermohydrosulfuricus, Thermoanaerobacter ethanolicus, Thermoanaerobacter brocki, Geobacillus thermoglucosidasius, Geobacillus stearothermophilus, Saccharococcus caldoxylosilyticus, Saccharoccus thermophilus, Paenibacillus campinasensis, Bacillus flavothermus, Anoxybacillus kamchatkensis, Anoxybacillus gonensis, Caldicellulosiruptor acetigenus, Caldicellulosiruptor saccharolyticus, Caldicellulosiruptor kristjanssonii, Caldicellulosiruptor owensensis, Caldicellulosiruptor lactoaceticus, Anaerocellum thermophilum, Clostridium thermocellum, Clostridium cellulolyticum, Clostridium straminosolvens, Clostridium acetobutylicum, Clostridium aerotolerans, Clostridium beijerinckii, Clostridium bifermentans, Clostridium botulinum, Clostridium butyricum, Clostridium cadaveric, Clostridium chauvoei, Clostridium clostridioforme, Clostridium colicanis, Clostridium difficile, Clostridium fallax, Clostridium formicaceticum, Clostridium histolyticum, Clostridium innocuum, Clostridium ljungdahlii, Clostridium laramie, Clostridium lavalense, Clostridium novyi, Clostridium oedematiens, Clostridium paraputrificum, Clostridium perfringens, Clostridium phytofermentans, Clostridium piliforme, Clostridium ramosum, Clostridium scatologenes, Clostridium septicum, Clostridium sordellii, Clostridium sporogenes, Clostridium tertium, Clostridium tetani, Clostridium tyrobutyricum, Clostridium thermobutyricum, Zymomonas mobilis, or a variant or genetically modified version thereof.

In one embodiment, fed-batch fermentation is performed on the treated biomass to produce a fermentation end-product, such as alcohol, ethanol, organic acid, succinic acid, a polyols (e.g., glycerol), a fatty acid, triacylglycerol (TAG), or hydrogen. In one embodiment, the fermentation process comprises simultaneous hydrolysis and fermentation (SSF) of the biomass using one or more microorganisms such as a Rhodococcus strain, a Clostridium strain, a Trichoderma strain, a Saccharomyces strain, a Zymomonas strain, or another microorganism suitable for fermentation of biomass. In another embodiment, the fermentation process comprises simultaneous hydrolysis and fermentation of the biomass using a microorganism that is Clostridium algidixylanolyticum, Clostridium xylanolyticum, Clostridium cellulovorans, Clostridium cellulolyticum, Clostridium thermocellum, Clostridium josui, Clostridium papyrosolvens, Clostridium cellobioparum, Clostridium hungatei, Clostridium cellulosi, Clostridium stercorarium, Clostridium termitidis, Clostridium thermocopriae, Clostridium celerecrescens, Clostridium polysaccharolyticum, Clostridium populeti, Clostridium lentocellum, Clostridium chartatabidum, Clostridium aldrichii, Clostridium herbivorans, Clostridium phytofermentans, Acetivibrio cellulolyticus, Bacteroides cellulosolvens, Caldicellulosiruptor saccharolyticum, Ruminococcus albus, Ruminococcus flavefaciens, Fibrobacter succinogenes, Eubacterium cellulosolvens, Butyrivibrio fibrisolvens, Anaerocellum thermophilum, Halocella cellulolytica, Thermoanaerobacterium thermosaccharolyticum, Sacharophagus degradans, or Thermoanaerobacterium saccharolyticum.

In one embodiment, the fermentation process can include separate hydrolysis and fermentation (SHF) of a biomass with one or more enzymes, such as a xylanases, endo-1,4-beta-xylanases, xylosidases, beta-D-xylosidases, cellulases, hemicellulases, carbohydrases, glucanases, endoglucanases, endo-1,4-beta-glucanases, exoglucanases, glucosidases, beta-D-glucosidases, amylases, cellobiohydrolases, exocellobiohydrolases, phytases, proteases, peroxidase, pectate lyases, galacturonases, or laccases. In one embodiment one or more enzymes used to treat a biomass is thermostable. In another embodiment a biomass is treated with one or more enzymes, such as those provided herein, prior to fermentation. In another embodiment a biomass is treated with one or more enzymes, such as those provided herein, during fermentation. In another embodiment a biomass is treated with one or more enzymes, such as those provided herein, prior to fermentation and during fermentation. In another embodiment an enzyme used for hydrolysis of a biomass is the same as those added during fermentation. In another embodiment an enzyme used for hydrolysis of biomass is different from those added during fermentation.

In some embodiments, fermentation can be performed in an apparatus such as bioreactor, a fermentation vessel, a stirred tank reactor, or a fluidized bed reactor. In one embodiment the treated biomass can be supplemented with suitable chemicals to facilitate robust growth of the one or more fermenting organisms. In one embodiment a useful supplement includes but is not limited to, a source of nitrogen and/or amino acids such as yeast extract, cysteine, or ammonium salts (e.g. nitrate, sulfate, phosphate etc.); a source of simple carbohydrates such as corn steep liquor, and malt syrup; a source of vitamins such as yeast extract; buffering agents such as salts (including but not limited to citrate salts, phosphate salts, or carbonate salts); or mineral nutrients such as salts of magnesium, calcium, or iron. In some embodiments redox modifiers are added to the fermentation mixture including but not limited to cysteine or mercaptoethanol.

In one embodiment, the titer and/or productivity of fermentation end-product production by a microorganism is improved by culturing the microorganism in a medium comprising one or more compounds comprising hexose and/or pentose sugars. In one embodiment, a process comprises conversion of a starting material (such as a biomass) to a biofuel, such as one or more alcohols. In one embodiment, methods can comprise contacting substrate comprising both hexose (e.g. glucose, cellobiose) and pentose (e.g. xylose, arabinose) saccharides with a microorganism that can hydrolyze C5 and C6 saccharides to produce ethanol. In another embodiment, methods can comprise contacting substrate comprising both hexose (e.g. glucose, cellobiose) and pentose (e.g. xylose, arabinose) saccharides with R. opacus to produce TAG.

In some embodiments, batch fermentation with a microorganism of a mixture of hexose and pentose saccharides using the methods disclosed herein can provide for uptake rates of about 0.1-8 g/L/h or more of hexose and about 0.1-8 g/L/h or more of pentose (xylose, arabinose, etc.). In some embodiments, batch fermentation with a microorganism of a mixture of hexose and pentose saccharides using the methods disclosed herein provide for uptake rates of about 0.1, 0.2, 0.4, 0.5, 0.6 0.7, 0.8, 1, 2, 3, 4, 5, or 6 g/L/h or more of hexose and about 0.1, 0.2, 0.4, 0.5, 0.6 0.7, 0.8, 1, 2, 3, 4, 5, or 6 g/L/h or more of pentose.

In one embodiment, a method for production of ethanol or another alcohol produces about 10 g/l to 120 gain 40 hours or less. In another embodiment a method for production of ethanol produces about 10 g/l, 11 g/L, 12 g/L, 13 g/L, 14 g/L, 15 g/L, 16 g/L, 17 g/L, 18 g/L, 19 g/L, 20 g/L, 21 g/L, 22 g/L, 23 g/L, 24 g/L, 25 g/L, 26 g/L, 27 g/L, 28 g/L, 29 g/L, 30 g/L, 31 g/L, 32 g/L, 33 g/L, 34 g/L, 35 g/L, 36 g/L, 37 g/L, 38 g/L, 39 g/L, 40 g/L, 41 g/L, 42 g/L, 43 g/L, 44 g/L, 45 g/L, 46 g/L, 47 g/L, 48 g/L, 49 g/L, 50 g/L, 51 g/L, 52 g/L, 53 g/L, 54 g/L, 55 g/L, 56 g/L, 57 g/L, 58 g/L, 59 g/L, 60 g/L, 61 g/L, 62 g/L, 63 g/L, 64 g/L, 65 g/L, 66 g/L, 67 g/L, 68 g/L, 69 g/L, 70 g/L, 71 g/L, 72 g/L, 73 g/L, 74 g/L, 75 g/L, 76 g/L, 77 g/L, 78 g/L, 79 g/L, 80 g/L, 81 g/L, 82 g/L, 83 g/L, 84 g/L, 85 g/L, 86 g/L, 87 g/L, 88 g/L, 89 g/L, 90 g/L, 91 g/L, 92 g/L, 93 g/L, 94 g/L, 95 g/L, 96 g/L, 97 g/L, 98 g/L, 99 g/L, 100 g/L, 110 g/l, 120 g/l, or more alcohol in 40 hours by the fermentation of biomass. In another embodiment, alcohol is produced by a method comprising simultaneous fermentation of hexose and pentose saccharides. In another embodiment, alcohol is produced by a microorganism comprising simultaneous fermentation of hexose and pentose saccharides.

In another embodiment, the level of a medium component is maintained at a desired level by adding additional medium component as the component is consumed or taken up by the organism. Examples of medium components included, but are not limited to, carbon substrate, nitrogen substrate, vitamins, minerals, growth factors, cofactors, and biocatalysts. The medium component can be added continuously or at regular or irregular intervals. In one embodiment, additional medium component is added prior to the complete depletion of the medium component in the medium. In one embodiment, complete depletion can effectively be used, for example to initiate different metabolic pathways, to simplify downstream operations, or for other reasons as well. In one embodiment, the medium component level is allowed to vary by about 10% around a midpoint, in one embodiment, it is allowed to vary by about 30% around a midpoint, and in one embodiment, it is allowed to vary by 60% or more around a midpoint. In one embodiment, the medium component level is maintained by allowing the medium component to be depleted to an appropriate level, followed by increasing the medium component level to another appropriate level. In one embodiment, a medium component, such as vitamin, is added at two different time points during fermentation process. For example, one-half of a total amount of vitamin is added at the beginning of fermentation and the other half is added at midpoint of fermentation.

In another embodiment, the nitrogen level is maintained at a desired level by adding additional nitrogen-containing material as nitrogen is consumed or taken up by the organism. The nitrogen-containing material can be added continuously or at regular or irregular intervals. Useful nitrogen levels include levels of about 5 to about 10 g/L. In one embodiment levels of about 1 to about 12 g/L can also be usefully employed. In another embodiment levels, such as about 0.5, 0.1 g/L or even lower, and higher levels, such as about 20, 30 g/L or even higher are used. In another embodiment a useful nitrogen level is about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22 23, 24, 25, 26, 27, 28, 29 or 30 g/L. Nitrogen can be supplied as a simple nitrogen-containing material, such as an ammonium compounds (e.g. ammonium sulfate, ammonium hydroxide, ammonia, ammonium nitrate, or any other compound or mixture containing an ammonium moiety), nitrate or nitrite compounds (e.g. potassium, sodium, ammonium, calcium, or other compound or mixture containing a nitrate or nitrite moiety), or as a more complex nitrogen-containing material, such as amino acids, proteins, hydrolyzed protein, hydrolyzed yeast, yeast extract, dried brewer's yeast, yeast hydrolysates, distillers' grains, soy protein, hydrolyzed soy protein, fermentation products, and processed or corn steep powder or unprocessed protein-rich vegetable or animal matter, including those derived from bean, seeds, soy, legumes, nuts, milk, pig, cattle, mammal, fish, as well as other parts of plants and other types of animals. Nitrogen-containing materials useful in various embodiments also include materials that contain a nitrogen-containing material, including, but not limited to mixtures of a simple or more complex nitrogen-containing material mixed with a carbon source, another nitrogen-containing material, or other nutrients or non-nutrients, and AFEX treated plant matter.

In another embodiment, the carbon level is maintained at a desired level by adding sugar compounds or material containing sugar compounds (“Sugar-Containing Material”) as sugar is consumed or taken up by the organism. The sugar-containing material can be added continuously or at regular or irregular intervals. In one embodiment, additional sugar-containing material is added prior to the complete depletion of the sugar compounds available in the medium. In one embodiment, complete depletion can effectively be used, for example to initiate different metabolic pathways, to simplify downstream operations, or for other reasons as well. In one embodiment, the carbon level (as measured by the grams of sugar present in the sugar-containing material per liter of broth) is allowed to vary by about 10% around a midpoint, in one embodiment, it is allowed to vary by about 30% around a midpoint, and in one embodiment, it is allowed to vary by 60% or more around a midpoint. In one embodiment, the carbon level is maintained by allowing the carbon to be depleted to an appropriate level, followed by increasing the carbon level to another appropriate level. In some embodiments, the carbon level can be maintained at a level of about 5 to about 120 g/L. However, levels of about 30 to about 100 g/L can also be usefully employed as well as levels of about 60 to about 80 g/L. In one embodiment, the carbon level is maintained at greater than 25 g/L for a portion of the culturing. In another embodiment, the carbon level is maintained at about 5 g/L, 6 g/L, 7 g/L, 8 g/L, 9 g/L, 10 g/L, 11 g/L, 12 g/L, 13 g/L, 14 g/L, 15 g/L, 16 g/L, 17 g/L, 18 g/L, 19 g/L, 20 g/L, 21 g/L, 22 g/L, 23 g/L, 24 g/L, 25 g/L, 26 g/L, 27 g/L, 28 g/L, 29 g/L, 30 g/L, 31 g/L, 32 g/L, 33 g/L, 34 g/L, 35 g/L, 36 g/L, 37 g/L, 38 g/L, 39 g/L, 40 g/L, 41 g/L, 42 g/L, 43 g/L, 44 g/L, 45 g/L, 46 g/L, 47 g/L, 48 g/L, 49 g/L, 50 g/L, 51 g/L, 52 g/L, 53 g/L, 54 g/L, 55 g/L, 56 g/L, 57 g/L, 58 g/L, 59 g/L, 60 g/L, 61 g/L, 62 g/L, 63 g/L, 64 g/L, 65 g/L, 66 g/L, 67 g/L, 68 g/L, 69 g/L, 70 g/L, 71 g/L, 72 g/L, 73 g/L, 74 g/L, 75 g/L, 76 g/L, 77 g/L, 78 g/L, 79 g/L, 80 g/L, 81 g/L, 82 g/L, 83 g/L, 84 g/L, 85 g/L, 86 g/L, 87 g/L, 88 g/L, 89 g/L, 90 g/L, 91 g/L, 92 g/L, 93 g/L, 94 g/L, 95 g/L, 96 g/L, 97 g/L, 98 g/L, 99 g/L, 100 g/L, 101 g/L, 102 g/L, 103 g/L, 104 g/L, 105 g/L, 106 g/L, 107 g/L, 108 g/L, 109 g/L, 110 g/L, 111 g/L, 112 g/L, 113 g/L, 114 g/L, 115 g/L, 116 g/L, 117 g/L, 118 g/L, 119 g/L, 120 g/L, 121 g/L, 122 g/L, 123 g/L, 124 g/L, 125 g/L, 126 g/L, 127 g/L, 128 g/L, 129 g/L, 130 g/L, 131 g/L, 132 g/L, 133 g/L, 134 g/L, 135 g/L, 136 g/L, 137 g/L, 138 g/L, 139 g/L, 140 g/L, 141 g/L, 142 g/L, 143 g/L, 144 g/L, 145 g/L, 146 g/L, 147 g/L, 148 g/L, 149 g/L, or 150 g/L.

The carbon substrate, like the nitrogen substrate, can be used for cell production and enzyme production. The carbons substrate can serve as the raw material for production of fermentation end-products. Frequently, more carbon substrate can lead to greater production of fermentation end-products. In another embodiment, it can be advantageous to operate with the carbon level and nitrogen level related to each other for at least a portion of the fermentation time. In one embodiment, the ratio of carbon to nitrogen is maintained within a range of about 30:1 to about 10:1. In another embodiment, the ratio of carbon nitrogen is maintained from about 20:1 to about 10:1, or from about 15:1 to about 10:1. In another embodiment the ratio of carbon nitrogen is about 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or 1:1.

Maintaining the ratio of carbon and nitrogen ratio within particular ranges can result in benefits to the operation such as the rate of metabolism of carbon substrate, which depends on the amount of carbon substrate and the amount and activity of enzymes present, being balanced to the rate of end product production. Balancing the carbon to nitrogen ratio can, for example, facilitate the sustained production of these enzymes such as to replace those which have lost activity.

In another embodiment, the amount and/or timing of carbon, nitrogen, or other medium component addition can be related to measurements taken during the fermentation. For example, the amount of monosaccharides present, the amount of insoluble polysaccharide present, the polysaccharase activity, the amount of product present, the amount of cellular material (for example, packed cell volume, dry cell weight, etc.) and/or the amount of nitrogen (for example, nitrate, nitrite, ammonia, urea, proteins, amino acids, etc.) present can be measured. The concentration of the particular species, the total amount of the species present in the fermentor, the number of hours the fermentation has been running, and the volume of the fermentor can be considered. In various embodiments, these measurements can be compared to each other and/or they can be compared to previous measurements of the same parameter previously taken from the same fermentation or another fermentation. Adjustments to the amount of a medium component can be accomplished such as by changing the flow rate of a stream containing that component or by changing the frequency of the additions for that component. For example, the amount of saccharide can be increased when the cell production increases faster than the end product production. In another embodiment the amount of nitrogen can be increased when the enzyme activity level decreases.

In another embodiment, a fed batch operation can be employed, wherein medium components and/or fresh cells are added during the fermentation without removal of a portion of the broth for harvest prior to the end of the fermentation. In one embodiment a fed-batch process is based on feeding a growth limiting nutrient medium to a culture of microorganisms. In one embodiment the feed medium is highly concentrated to avoid dilution of the bioreactor. In another embodiment the controlled addition of the nutrient directly affects the growth rate of the culture and avoids overflow metabolism such as the formation of side metabolites. In one embodiment the growth limiting nutrient is a nitrogen source or a saccharide source.

In various embodiments, particular medium components can have beneficial effects on the performance of the fermentation, such as increasing the titer of desired products, or increasing the rate that the desired products are produced. Specific compounds can be supplied as a specific, pure ingredient, such as a particular amino acid, or it can be supplied as a component of a more complex ingredient, such as using a microbial, plant or animal product as a medium ingredient to provide a particular amino acid, promoter, cofactor, or other beneficial compound. In some cases, the particular compound supplied in the medium ingredient can be combined with other compounds by the organism resulting in a fermentation-beneficial compound. One example of this situation would be where a medium ingredient provides a specific amino acid which the organism uses to make an enzyme beneficial to the fermentation. Other examples can include medium components that are used to generate growth or product promoters, etc. In such cases, it can be possible to obtain a fermentation-beneficial result by supplementing the enzyme, promoter, growth factor, etc. or by adding the precursor. In some situations, the specific mechanism whereby the medium component benefits the fermentation is not known, only that a beneficial result is achieved.

In one embodiment, a fermentation to produce a fuel is performed by culturing a strain of R. opacus in a medium having a supplement of lignin component and a concentration of one or more carbon sources. The resulting production of end product such as TAG can be up to 1-fold, 2-fold, 3-fold, 4-fold, 5-fold, 6-fold, 7-fold, 8-fold, 9-fold, and in some cases up to 10-fold and higher in volumetric productivity than a process using only the addition of a relatively pure saccharide source, and can achieve a carbon conversion efficiency approaching the theoretical maximum. The theoretical maximum can vary with the substrate and product. For example, the generally accepted maximum efficiency for conversion of glucose to ethanol is 0.51 g ethanol/g glucose. In one embodiment a biocatalyst can produce about 40-100% of a theoretical maximum yield of ethanol. In another embodiment, a biocatalyst can produce up to about 40%, 50%, 60%, 70%, 80%, 90%, 95% and even 100% of the theoretical maximum yield of ethanol. In one embodiment a biocatalyst can produce up to about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.99%, or 100% of a theoretical maximum yield of a fuel. It can be possible to obtain a fermentation-beneficial result by supplementing the medium with a pretreatment or hydrolysis component. In some situations, the specific mechanism whereby the medium component benefits the fermentation is not known, only that a beneficial result is achieved.

Various embodiments offer benefits relating to improving the titer and/or productivity of fermentation end-product production by a biocatalyst by culturing the organism in a medium comprising one or more compounds comprising particular fatty acid moieties and/or culturing the organism under conditions of controlled pH.

In one embodiment, the pH of the medium is controlled at less than about pH 7.2 for at least a portion of the fermentation. In one embodiment, the pH is controlled within a range of about pH 3.0 to about 7.1 or about pH 4.5 to about 7.1, or about pH 5.0 to about 6.3, or about pH 5.5 to about 6.3, or about pH 6.0 to about 6.5, or about pH 5.5 to about 6.9 or about pH 6.2 to about 6.7. The pH can be controlled by the addition of a pH modifier. In one embodiment, a pH modifier is an acid, a base, a buffer, or a material that reacts with other materials present to serve to raise of lower the pH. In one embodiment, more than one pH modifier can be used, such as more than one acid, more than one base, one or more acid with one or more bases, one or more acids with one or more buffers, one or more bases with one or more buffers, or one or more acids with one or more bases with one or more buffers. When more than one pH modifiers are utilized, they can be added at the same time or at different times. In one embodiment, one or more acids and one or more bases can be combined, resulting in a buffer. In one embodiment, media components, such as a carbon source or a nitrogen source can also serve as a pH modifier; suitable media components include those with high or low pH or those with buffering capacity. Exemplary media components include acid- or base-hydrolyzed plant polysaccharides having with residual acid or base, AFEX treated plant material with residual ammonia, lactic acid, corn steep solids or liquor.

In one embodiment, a constant pH can be utilized throughout the fermentation. In one embodiment, the timing and/or amount of pH reduction can be related to the growth conditions of the cells, such as in relation to the cell count, the end product produced, the end product present, or the rate of end product production. In one embodiment, the pH reduction can be made in relation to physical or chemical properties of the fermentation, such as viscosity, medium composition, gas production, off gas composition, etc.

Recovery of Fermentation End-Products

In another aspect, methods are provided for the recovery of the fermentive end products, such as an alcohol (e.g. ethanol, propanol, methanol, butanol, etc.) another biofuel or chemical product. In one embodiment, broth will be harvested at some point during of the fermentation, and fermentive end product or products will be recovered. The broth with end product to be recovered will include both end product and impurities. The impurities include materials such as water, cell bodies, cellular debris, excess carbon substrate, excess nitrogen substrate, other remaining nutrients, other metabolites, and other medium components or digested medium components. During the course of processing the broth, the broth can be heated and/or reacted with various reagents, resulting in additional impurities in the broth.

In one embodiment, the processing steps to recover end product frequently includes several separation steps, including, for example, distillation of a high concentration alcohol material from a less pure alcohol-containing material. In one embodiment, the high concentration alcohol material can be further concentrated to achieve very high concentration alcohol, such as 98% or 99% or 99.5% (wt.) or even higher. Other separation steps, such as filtration, centrifugation, extraction, adsorption, etc. can also be a part of some recovery processes for alcohol as a product or biofuel, or other biofuels or chemical products.

In one embodiment a process can be scaled to produce commercially useful biofuels. In another embodiment biocatalyst is used to produce an alcohol, e.g., ethanol, butanol, propanol, methanol, or a fuel such as hydrocarbons hydrogen, TAG, and hydroxy compounds. In another embodiment biocatalyst is used to produce a carbonyl compound such as an aldehyde or ketone (e.g. acetone, formaldehyde, 1-propanal, etc.), an organic acid, a derivative of an organic acid such as an ester (e.g. wax ester, glyceride, etc.), 1,2-propanediol, 1,3-propanediol, lactic acid, formic acid, acetic acid, succinic acid, pyruvic acid, or an enzyme such as a cellulase, polysaccharase, lipases, protease, ligninase, and hemicellulase.

TAG biosynthesis is widely distributed in nature and the occurrence of TAG as reserve compounds is widespread among plants, animals, yeast and fungi. In contrast, however, TAGs have not been regarded as common storage compounds in bacteria. Biosynthesis and accumulation of TAGs have been described only for a few bacteria belonging to the actinomycetes group, such as genera of Streptomyces, Nocardia, Rhodococcus, Mycobacterium, Dietzia and Gordonia, and, to a minor extent, also in a few other bacteria, such as Acinetobacter baylyi and Alcanivorax borkumensis. Since the mid-1990's, TAG production in hydrocarbon-degrading strains of those genera has been frequently reported. TAGs are stored in spherical lipid bodies as intracellular inclusions, with the amounts depending on the respective species, cultural conditions and growth phase. Commonly, the important factor for the production of TAGs is the amount of nitrogen that is supplied to the culture medium. The excess carbon, which is available to the culture after nitrogen exhaustion, continues to be assimilated by the cells and, by virtue of oleaginous bacteria possessing the requisite enzymes, is converted directly into lipid. The compositions and structures of bacterial TAG molecules vary considerably depending on the bacterium and on the cultural conditions, especially the carbon sources. See, Brigham C J, et al. (2011) J Microbial Biochem Technol S3:002.

In one embodiment, useful biochemicals can be produced from non-food plant biomass, with a steam or hot-water extraction technique that is carried out by contacting a charge of non-food plant pretreated biomass material such as corn stover or sorhum with water and/or acid (with or without additional process enhancing compounds or materials), in a pressurized vessel at an elevated temperature up to about 160-220° C. and at a pH below about 7.0, to yield an aqueous (extract solution) mixture of useful sugars including long-chain saccharides (sugars), acetic acid, and lignin, while leaving the structural (cellulose and lignin) portion of the lignocellulosic material largely intact. In combination, these potential inhibitory chemicals, especially sugar degradation products, are low, and the plant derived nutrients that are naturally occurring lignocellulosic-based components are also recovered that are beneficial to a C5 and C6 fermenting organism. Toward this objective, the aqueous extract is concentrated (by centrifugation, filtration, solvent extraction, flocculation, evaporation), by producing a concentrated sugar stream, apart from the other hemicellulose (C5 rich) and cellulosic derived sugars (C6 rich) which are channeled into a fermentable stream.

In another embodiment, following enzyme/acid hydrolysis, additional chemical compounds that are released are recovered with the sugar stream resulting in a short-chain sugar solution containing xylose, mannose, arabinose, rhamnose, galactose, and glucose (5 and 6-carbon sugars). The sugar stream, now significantly rich in C5 and C6 substances can be converted by microbial fermentation or chemical catalysis into such products as triacylglycerol or TAG and further refined to produce stream rich in JP8 or jet fuels. If C5 sugar percentage correction has not been performed, it can be performed before fermentation to satisfy desired combination of C5 and C6 sugars for the fermentation organism and corresponding end product.

Biofuel Plant and Process of Producing Biofuel:

Large Scale Fuel and Chemical Production from Biomass

Generally, there are several basic approaches to producing fuels and chemical end-products from biomass on a large scale utilizing of microbial cells. In the one method, one first pretreats and hydrolyzes a biomass material that includes high molecular weight carbohydrates to lower molecular weight carbohydrates, and then ferments the lower molecular weight carbohydrates utilizing of microbial cells to produce fuel or other products. In the second method, one treats the biomass material itself using mechanical, chemical and/or enzymatic methods. In all methods, depending on the type of biomass and its physical manifestation, one of the processes can comprise a milling of the carbonaceous material, via wet or dry milling, to reduce the material in size and increase the surface to volume ratio (physical modification).

In one embodiment, hydrolysis can be accomplished using acids, e.g., Bronsted acids (e.g., sulfuric or hydrochloric acid), bases, e.g., sodium hydroxide, hydrothermal processes, ammonia fiber explosion processes (“AFEX”), lime processes, enzymes, or combination of these. Hydrogen, and other end products of the fermentation can be captured and purified if desired, or disposed of, e.g., by burning. For example, the hydrogen gas can be flared, or used as an energy source in the process, e.g., to drive a steam boiler, e.g., by burning. Hydrolysis and/or steam treatment of the biomass can, e.g., increase porosity and/or surface area of the biomass, often leaving the cellulosic materials more exposed to the biocatalyst cells, which can increase fermentation rate and yield. Removal of lignin can, e.g., provide a combustible fuel for driving a boiler, and can also, e.g., increase porosity and/or surface area of the biomass, often increasing fermentation rate and yield. Generally, in any of the these embodiments, the initial concentration of the carbohydrates in the medium is greater than 20 mM, e.g., greater than 30 mM, 50 mM, 75 mM, 100 mM, 150 mM, 200 mM, or even greater than 500 mM.

Biomass Processing Plant and Process of Producing Products from Biomass

In one aspect, a fuel or chemical plant that includes a pretreatment unit to prepare biomass for improved exposure and biopolymer separation, a hydrolysis unit configured to hydrolyze a biomass material that includes a high molecular weight carbohydrate, and one or more product recovery system(s) to isolate a product or products and associated by-products and co-products is provided. In another aspect, methods of purifying lower molecular weight carbohydrates from solid byproducts and/or toxic impurities are provided.

In another aspect, methods of making a product or products that include combining biocatalyst cells of a microorganism and a biomass feed in a medium wherein the biomass feed contains lower molecular weight carbohydrates and unseparated solids and/or other liquids from pretreatment and hydrolysis, and fermenting the biomass material under conditions and for a time sufficient to produce a biofuel, chemical product or fermentive end-products, e.g. ethanol, propanol, hydrogen, succinic acid, lignin, terpenoids, and the like as described above, is provided.

In another aspect, products made by any of the processes described herein are also provided herein.

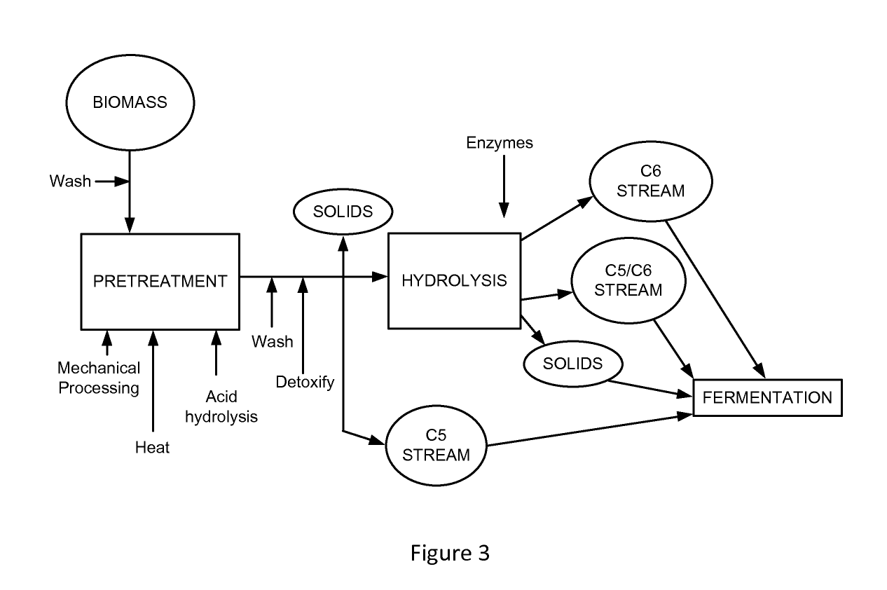

FIG. 3 is an example of a method for producing chemical products from biomass by first treating biomass with an acid at elevated temperature and pressure in a hydrolysis unit. The biomass may first be heated by addition of hot water or steam. The biomass may be acidified by bubbling gaseous sulfur dioxide through the biomass that is suspended in water, or by adding a strong acid, e.g., sulfuric, hydrochloric, or nitric acid with or without preheating/presteaming/water addition. During the acidification, the pH is maintained at a low level, e.g., below about 5. The temperature and pressure may be elevated after acid addition. In addition to the acid already in the acidification unit, optionally, a metal salt such as ferrous sulfate, ferric sulfate, ferric chloride, aluminum sulfate, aluminum chloride, magnesium sulfate, or mixtures of these can be added to aid in the acid hydrolysis of the biomass. The acid-impregnated biomass is fed into the hydrolysis section of the pretreatment unit. Steam is injected into the hydrolysis portion of the pretreatment unit to directly contact and heat the biomass to the desired temperature. The temperature of the biomass after steam addition is, e.g., between about 130° C. and 220° C. The acid hydrolysate is then discharged into the flash tank portion of the pretreatment unit, and is held in the tank for a period of time to further hydrolyze the biomass, e.g., into oligosaccharides and monomeric sugars. Other methods can also be used to further break down biomass. Alternatively, the biomass can be subject to discharge through a pressure lock for any high-pressure pretreatment process. Hydrolysate is then discharged from the pretreatment reactor, with or without the addition of water, e.g., at solids concentrations between about 10% and 60%.

After pretreatment, the biomass may be dewatered and/or washed with a quantity of water, e.g. by squeezing or by centrifugation, or by filtration using, e.g. a countercurrent extractor, wash press, filter press, pressure filter, a screw conveyor extractor, or a vacuum belt extractor to remove acidified fluid. Wash fluids can be collected to concentrate the C5 saccharides in the wash stream. The acidified fluid, with or without further treatment, e.g. addition of alkali (e.g. lime) and or ammonia (e.g. ammonium phosphate), can be re-used, e.g., in the acidification portion of the pretreatment unit, or added to the fermentation, or collected for other use/treatment. Products may be derived from treatment of the acidified fluid, e.g., gypsum or ammonium phosphate. Enzymes or a mixture of enzymes can be added during pretreatment to hydrolyze, e.g. endoglucanases, exoglucanases, cellobiohydrolases (CBH), beta-glucosidases, glycoside hydrolases, glycosyltransferases, alphyamylases, chitinases, pectinases, lyases, and esterases active against components of cellulose, hemicelluloses, pectin, and starch, in the hydrolysis of high molecular weight components.

A fermentor, attached or at a separate site, can be fed with hydrolyzed biomass, any liquid fraction from biomass pretreatment, an active seed culture of a biocatalyst, such as a yeast, if desired a co-fermenting microbe, e.g., another yeast or E. coli, and, if required, nutrients to promote growth of the biocatalyst or other microbes. Alternatively, the pretreated biomass or liquid fraction can be split into multiple fermentors, each containing a different strain of a biocatalyst and/or other microbes, and each operating under specific physical conditions. Fermentation is allowed to proceed for a period of time, e.g., between about 1 and 150 hours, while maintaining a temperature of, e.g., between about 25° C. and 50° C. Gas produced during the fermentation is swept from fermentor and is discharged, collected, or flared with or without additional processing, e.g. hydrogen gas may be collected and used as a power source or purified as a co-product.

In another aspect, methods of making a fuel or fuels that include combining one or more biocatalyst and a lignocellulosic material (and/or other biomass material) in a medium, adding a lignin fraction from pretreatment, and fermenting the lignocellulosic material under conditions and for a time sufficient to produce a fuel or fuels, e.g., ethanol, propanol and/or hydrogen or another chemical compound is provided herein.

In another aspect, the products made by any of the processes described herein is provided.

### EXAMPLES

The following examples serve to illustrate certain embodiments and aspects and are not to be construed as limiting the scope thereof.

### Example 1

### Pretreatment and Saccharification of Biomass

Three different samples of energy sorghum sileage were prepared by drying, grinding, and compression. This material was suspended in 1% NaOH and incubated at 90° C., then filtered and washed. This pretreated material was suspended in 100 ml water and the pH adjusted to 5.0 with sulfuric acid. Saccharification on the partially-delignified residue was carried out at 45° C. at 200 rpm with four different enzymes: 4 ml viscozyme, 2 ml cellulase C2730, 0.5 ml novozyme 188 and 0.5 ml glucoamylase A7095 (Novozymes A/S, Krogshoejvej 36, 2880 Bagsvaerd Denmark). A summary of the concentration and composition of the resulting sugars is provided below in Table 1.

TABLE 1
Energy sorghum silage

| | Source A | | Source B | | Source C | |
|---|---|---|---|---|---|---|
| Moisture | 73% | | 82% | | 82% | |
| [Delignification] | | | | | | |
| Initial dried (wet) mass | 10(37) | g | 10(56) | g | 10(56) | g |
| Dried mass after | 4.4 | g | 5.9 | g | 4.6 | g |
| delignification | | | | | | |
| [Saccharification] | | | | | | |
| Total Sugars after | 2.9 | g | 3.1 | g | 3.0 | g |
| saccharification | | | | | | |
| Sugar concentration | 19 | g/L | 17 | g/l | 19 | g/L |
| Sugar composition | 11 | g/L | 10 | g/L | 11 | g/L |
| (Glucose) | | | | | | |
| (Xylose) | 6 | g/L | 5 | g/L | 6 | g/L |
| (Others) | 2 | g/L | 2 | g/L | 2 | g/L |

| | | | |
|---|---|---|---|
| [Sugar conversion] | 66% | 53% | |
| From dried (wet) | 29(7.8)% | 31(5.5)% | 30(5.4)% |
| silage | | | |
| From material after | 66% | 53% | 65% |
| delignification | | | |

Corn stover biomass was also prepared in accordance with a similar protocol. The NaOH treated and washed corn silage was suspended in dilute HCl, and 0.2-0.5 mL/g, as ml enzyme per gram pretreated material of Optimash XL (Genencor, Rochester, N.Y.) was added to the corn suspension and adjusted to pH5.0. Enzyme treatment was carried out at 45° C. at 200 rpm.

Other pretreatment methods were used to prepare energy sorghum and corn stover silage that did not involve initial caustic removal of lignin. For example, the water content of chopped, ensiled energy sorghum and corn stover was determined and adjusted to a solids content of about 15% [wt/v] solids and moisture content of about 85% [wt/v] using a 24 hour soaking treatment.

The moisture-adjusted corn stover and energy sorghum feedstocks were prepared for an acid-catalyzed steam or hot water pretreatment by impregnating the feedstocks with 1% H₂SO₄ (w/w based on dry weight) in a pressurized vessel at an elevated temperature up to about 160-220° C. and keeping a pH below 7 to yield an aqueous mixture of useful sugars including long-chain saccharides, acetic acid, and lignin, while leaving the structural (cellulose and lignin) portion of the lignocellulosic material largely intact, thus reducing the amount of inhibitory substances.

The impregnated raw material can also charged to a 60 L pressurized steam explosion batch reactor, at a temperature of about 200° C., a pH of about 2.9, for a period of time of 7.5 min, so that an aqueous extract (or liquor) containing solubilized components of the lignocellulosic material were obtained. The lignocellulosic slurries were adjusted to a pH of about 5 using 0.1 N NaOH. An enzyme cocktail (CELLIC CTech 2, Novozymes) was then added to the solid slurry. The amount of the enzyme added to the mixture was 2% loading (v/wt) based on the dry weight of the solids.

Once enzymatic hydrolysis was complete, the liquid slurry was separated by centrifugation or microfiltration; or, alternatively, the solids remained in the broth. For the experiments herein, the solids were left with the broth to produce a C6:C5 solution to be concentrated. About 30 L of the resultant C6-rich liquid slurry was concentrated by simple evaporation at a temperature of from about 70° C. to about 80° C. until the sugar content of the sorghum or corn hydrolysate was raised from about 5% to about 20% w/v. The resulting composition had a C6 sugar:C5 sugar ratio of about 90:10.

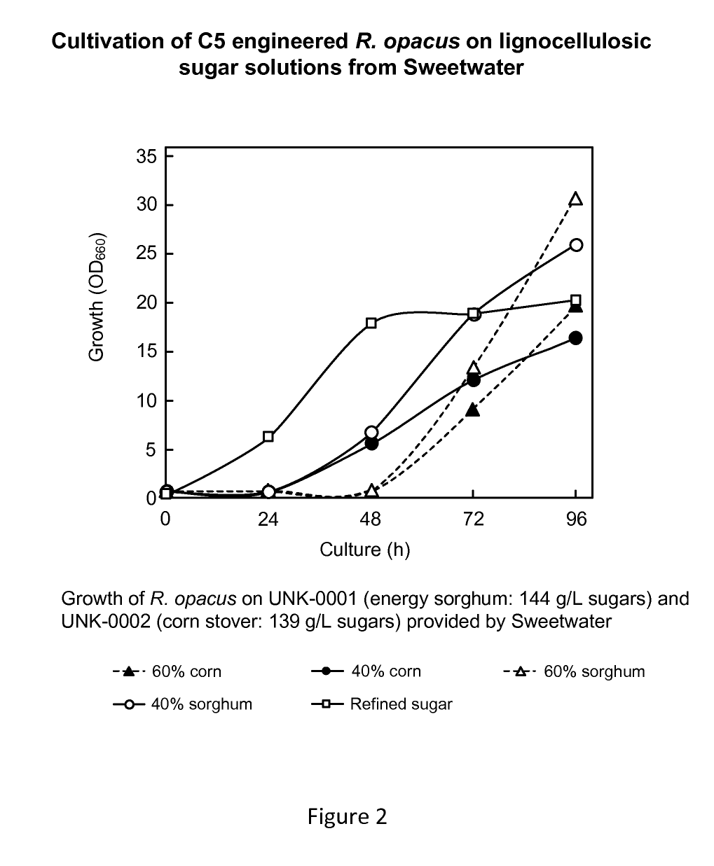

Analysis showed the total sugar composition in the undiluted solution from corn silage was 38.8 g/L. The sugars were composed of 13.8 g/L disaccharide, 20.9 g/L glucose, 2.9 g/L xylose, 0.9 g/L rhamnose and 0.3 g/L arabinose. The solution was adjusted to pH 7.2. Ammonium sulfate of 0.04 or 0.08 g was added into 5 ml water containing defined medium components (minerals), and it was mixed into the 45 ml of the solution. As a control (no addition), 5 ml water was added into 45 ml of the solution. After autoclaving, PD630 (wild type strain) or Xsp8 (xylose utilizable engineered strain) was grown on those media at 30° C. for 4 days. FIG. 2 shows the growth of the R. opacus strain on the lignocellulosic and refined sugars.

Both strains grew well, and growth inhibition was not observed on those media (TAG production on those media in both strains was good, the fatty acid content of dried cell mass was 36˜44%. The solution contained enough nutrients for PD630, but slightly lacked in nutrients for Xsp8 (DATA 3). The conversion from sugars to TAG on the corn solution was 13˜18% (lower level in comparison with at on sorghum). See Table 2. The microorganism grew well without defined media components only when the C6:C5 unseparated solution was used as a medium. Adding some additional ammonium sulfate increased growth somewhat.

TABLE 2
Lipid production of PD630 on the corn silage solution

| | | | Fatty acid | | Residual | |
|---|---|---|---|---|---|---|
| Medium (feedstock) | | Growth | production | | sugars | |
| Solution | g/L of sugars | DCM, g/L | %/DCM | g/L | g/L of sugars | Conversion* % |
| 50% corn silage** | 68 | 13.6 | 55.3 | 7.5 | 32 | 21 |
| 60% corn silage** | 82 | 14.7 | 53.6 | 7.9 | 44 | 21 |
| 70% corn silage** | 95 | 17.0 | 52.0 | 8.8 | 53 | 21 |
| Sorghum¹ | 40 | 18.1 | 47.4 | 8.6 | 1 | 22 |
| Sorghum² | 58 | 22.1 | 50.1 | 11.1 | 2 | 20 |

*Total fatty acid production/total consumed sugars in the solution.
**The corn feedstock was diluted 2 fold (50% solution), 5/3 fold (60% solution) or 10/7 (70% solution).
¹50 g/L of sorghum mass was saccharified.
²50 g/L of sorghum mass was saccharified and 20 g/L of glucose was added into the solution.

In comparison with sugarcane syrup (100% sucrose) or molasses (sucrose, glucose, fructose), corn stover, corn silage, or sorghum syrup produced by these methods was superior in growth and fatty acid (TAG) production during fermentation with R. opacus.

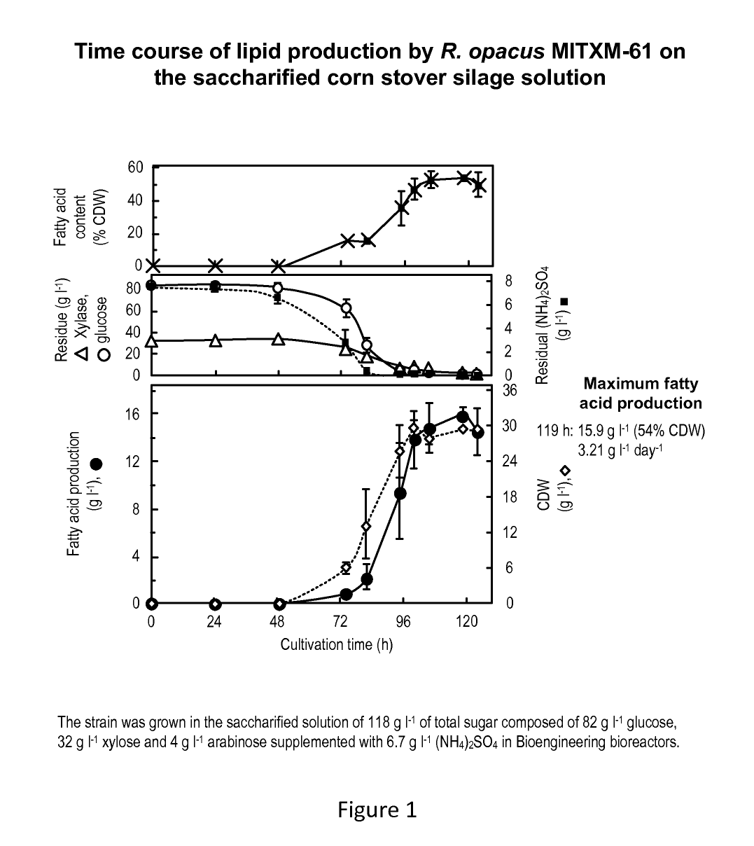

The data in FIG. 1 show the successful production of TAG precursor for jet fuel JP8 from pretreated and hydrolyzed corn stover silage having a mixture of C5 and C6 sugars.

The data in Table 3 shows that both the pretreated and hydrolyzed feedstocks produce superior TAG or % FA over the control mineral media or refined xylose/glucose sugar mixture. The 40% sorghum hydrolysate provides enough nutrients to support growth and fatty acid production in R. opacus and thus do not require mineral/nutrient supplementation.

TABLE 3
Lipid production of R. opacus on sorghum and stover

| | N¹, g/L | | Sugars, g/L | | | | CDW | FA | tFA | |
|---|---|---|---|---|---|---|---|---|---|---|
| Sample | Initial | Residual | Initial | Residual | Consumed | pH | g/L | content % | g/L | Conversion² % |
| 60% corn (+)³ | 1.7 | 0.0 | 82 | 61 | 21 | 5.1 | 11.7 | 34 | 4.0 | 19 |
| 40% corn (+)³ | 1.5 | 0.0 | 55 | 29 | 26 | 5.5 | 11.1 | 47 | 5.2 | 20 |
| 40% sorghum | 1.0 | 0.0 | 56 | 39 | 17 | 6.5 | 8.6 | 48 | 4.1 | 24 |
| 40% sorghum (+)³ | 2.0 | 0.0 | 55 | 43 | 12 | 4.5 | 7.7 | 16 | 1.2 | 10 |
| Refined sugar⁴ | 1.0 | 0.0 | 16 | 0 | 16 | 6.2 | 5.8 | 43 | 2.5 | 16 |

¹as (NH₄)₂SO₄
²sugar to FA
³minerals and 1 g/L (NH₄)₂SO₄
⁴defined medium containing 12 g/L glucose and 6 g/L xylose

### Example 2

### Cellulosic Sugar Solution from Delignified Corn Silage

The corn silage was provided with a few modifications of the original protocol for saccharification that were made to increase the sugar concentration and eliminate acetate buffer as an inhibitor for the growth. The corn silage (400 g wet mass)=280 g dry @ 70% moisture was suspended in 2 L of 1% NaOH (20 g of NaOH/280 g dry mass=7% NaoH loading) and incubated at 90° C. After 45 min of incubation, the suspension was filtered and the residue was washed with water (1 L). The material was resuspended in fresh 1% NaOH (2 L) at 7% NaOH loading and incubated at 90° C. After 30 min of incubation, the suspension was filtered and the residue was washed with water (3 L). The material was dried at 60° C. and afterwards milled by a homogenizer at 16000 rpm for 5 min. An aliquot of 20 g pretreated material was suspended in 180 mL water (11% solids) and the pH was adjusted to 5.0 with 1N HCl. Enzymes (8 mL viscozyme, 4 mL cellulase C2730 and 1 mL novozyme 188) were added to the suspension, and incubated at 45° C. with 200 rpm. After 8 and 24 h of incubation, additional pretreated material (10 g and 6 g respectively) equaling 20+10+6 g 36 g of biomass/˜200 g of water to produce 20% solids of biomass were used for hydrolysis. After 72 h of incubation, the total amount of released sugar was 112 g/L and the composition profile was 82 g/L glucose, 33 g/L xylose, 6 g/L arabinose and 1 g/L rhamnose. FIG. 2 shows TAG productivity by a C5 fermenting strain of R. opacus using sorghum, corn stover, and a control of refined sugar mixture.

### Example 3

In this example, the effect of particulate solids (e.g., residual solids, e.g., lignin particles) on the production of glycerol during fermentation with yeast was examined. Briefly, hardwood was pretreated and hydrolyzed to produce a sugar stream containing glucose, xylose, and arabinose and residual solids including particulate lignin. In one condition, lignin particulates were separated from the sugar stream (“Hardwood with solids removed”). In another condition, the lignin particulates were not removed prior to fermentation (“Hardwood”). A mixture of pure glucose and xylose was used as a control (“Refined sugar control”).

Propagation of Oe-2-1 120601 Yeast

50 mL of a stock solution containing 10% yeast extract, 1% MgSO₄ and 0.2% peptone was added to a 1 L baffled flask. 50 mL of a stock 40% dextrose solution was added to the flask also. Finally, the total volume of the solution was adjusted up to 500 mL with sterile DI water.

From the freezer, a thawed agar slant that had been inoculated with the 0e-2-1 120601 yeast (contains about 1 mL of inoculum). The slant was then placed in a 50° C. incubator for 25 minutes. Once the slant was thawed and warm to the touch, 4 mL of the propagation media was added to suspend the inoculum. The inoculum solution was poured back into the propagation flask and placed into the incubator. The incubator was maintained at 33° C. at 150 RPM for 24 hours throughout the course of the propagation.

Preparation of Hardwood for Fermentation

Hardwood, was pretreated and washed thoroughly with hot water to extract the C5 sugars. The temperature used for the hot water extraction was 60° C. The contact time was 30 minutes and the solids were washed using a continuous rinse line contained within the filter press. The solids were then hydrolyzed for 72 hours in a 50° C. jacketed vessel with agitating impellar. The total solids concentration was brought to 10% (wt/v). Cellulase and hemicellulase enzymes from Novozymes were used to break down the oligosaccharides into monomeric form. A 5 L portion of the hardwood sugars was sequestered and kept aside after hydrolysis was complete (Hardwood). The particulate solids, including lignin, were sequestered from the remaining 120 L of the hardwood sugars (Hardwood with solids removed). Both sugar solutions were centrifuged to remove excess lignin. Removal of the particulate solids resulted in a 0.48% drop in mass of the solution. The two sugar solutions were then concentrated via evaporation to about 20% total sugars (wt/v). Analysis was done on the HPLC to validate the sugar concentration.

Fermentation of Hardwood and Pure Sugar Streams with 0e-2-1 120601 Yeast

A pure glucose/xylose sugar solution was made to mimic the hardwood sugar solutions based on the HPLC data generated from the Hardwood with solids removed. 100 mL of each of the solutions were prepared in 250 mL shake flasks. The pH of all solutions was adjusted to 5.0+/−0.1 using sodium hydroxide. 250 uL of a 20% stock urea solution was also added to each flask.

The dry weight of yeast (in grams) per 5 mL of propagation solution was determined using an OHAUS MB35 moisture analyzer. Using a proportion based on the number expressed on the moisture analyzer, the amount of solution needed to inoculate four flasks with 0.025 g of dry yeast/25 mL of fermentation broth was calculated. The yeast propagation was spun down using a centrifuge set at 6000 RPM for 5 minutes. The supernatant was poured off and the yeast were suspended with 1 mL of sterile water. The yeast solution was then sub-divided evenly among the 4 fermentation flasks. Each flask was capped with a bubbler and placed in the incubator at 33° C. Samples were taken throughout the fermentation to observe the rate kinetics. Samples were taken from the fermentation reactions at 0, 8, 24, 32, 48 and 72 hours and analyzed for glucose, xylose, arabinose, glycerol, acetic acid, ethanol, hydroxymethylfurfural (HMF), and furfural. The results are shown in Tables 4 and 5.

TABLE 4
Fermentation Progress

| | | | | | Acetic | | | |
|---|---|---|---|---|---|---|---|---|
| | Glucose | Xylose | Arabinose | Glycerol | Acid | Ethanol | HMF* | Furfural |
| Sample Description | (g/L) | (g/L) | (g/L) | (g/L) | (g/L) | (g/L) | (g/L) | (g/L) |
| Hardwood with | 188.45 | 10.6 | 0.3 | 0.0 | 1.25 | 0 | 0.05 | 0 |
| solids removed 0 | | | | | | | | |
| hour | | | | | | | | |
| Hardwood with | 169.1 | 10.9 | 0.4 | 1.1 | 1.3 | 7.6 | 0 | 0 |
| solids removed T8 | | | | | | | | |
| Hardwood with | 100.05 | 9.8 | 0.4 | 4.3 | 1.2 | 40.4 | 0 | 0 |
| solids removed T24 | | | | | | | | |
| Hardwood with | 74.85 | 9.15 | 0.45 | 4.9 | 1.2 | 51.4 | 0 | 0 |
| solids removed T32 | | | | | | | | |
| Hardwood with | 33.6 | 9.1 | 0.1 | 5.1 | 1.15 | 72.45 | 0 | 0 |
| solids removed T48 | | | | | | | | |
| Hardwood with | 0.7 | 4.35 | 0.4 | 5.6 | 1.3 | 84.35 | 0 | 0 |
| solids removed T72 | | | | | | | | |
| Hardwood | 191.85 | 11.1 | 0.35 | 0.0 | 1.35 | 0 | 0.05 | 0 |
| 0 hour | | | | | | | | |
| Hardwood | 162.75 | 10.9 | 0.35 | 1.2 | 1.3 | 8.45 | 0 | 0 |
| T8 | | | | | | | | |
| Hardwood | 95.7 | 9.6 | 0.4 | 4.4 | 1.3 | 39.8 | 0 | 0 |
| T24 | | | | | | | | |
| Hardwood | 74.25 | 9.25 | 0.45 | 5.4 | 1.35 | 51.45 | 0 | 0 |
| T32 | | | | | | | | |
| Hardwood | 34.9 | 7.95 | 0.45 | 5.7 | 1.3 | 71.05 | 0 | 0 |
| T48 | | | | | | | | |
| Hardwood | 0.8 | 4.55 | 0.35 | 5.9 | 1.35 | 88.75 | 0 | 0 |
| T72 | | | | | | | | |
| Refined Sugar | 176.15 | 21.35 | 0 | 0.00 | 0 | 0 | 0 | 0 |
| Control | | | | | | | | |
| 0 hour | | | | | | | | |
| Refined Sugar | 148.35 | 20 | 0 | 1.92 | 0.35 | 7.95 | 0 | 0 |
| Control | | | | | | | | |
| T8 | | | | | | | | |
| Refined Sugar | 114.6 | 18.85 | 0 | 4.94 | 1.05 | 23.15 | 0 | 0 |
| Control | | | | | | | | |
| T24 | | | | | | | | |
| Refined Sugar | 102.85 | 18.35 | 0 | 5.83 | 1.25 | 28.65 | 0 | 0 |
| Control | | | | | | | | |
| T32 | | | | | | | | |
| Refined Sugar | 81.05 | 18.1 | 0 | 6.57 | 1.5 | 40.6 | 0 | 0 |
| Control | | | | | | | | |
| T48 | | | | | | | | |
| Refined Sugar | 44.95 | 15.7 | 0 | 8.05 | 1.65 | 59.5 | 0 | 0 |
| Control | | | | | | | | |
| T72 | | | | | | | | |

*HMF = Hydroxymethylfurfural

TABLE 5
Fermentation Summary

| | Initial | Ending | Initial | Ending | | Conv. | |
|---|---|---|---|---|---|---|---|
| Sample | Glucose | Glucose | Xylose | Xylose | Ethanol | Effic. | Glycerol |
| Description | (g/L) | (g/L) | (g/L) | (g/L) | (g/L) | (%) | (g/L) |
| Hardwood | 191.9 | 0.7 | 11.1 | 4.55 | 88.7 | 89.39 | 5.9 |
| Hardwood with | 188.4 | 0.8 | 10.6 | 4.35 | 86.35 | 88.72 | 5.6 |
| solids removed | | | | | | | |
| Refined Sugar Control | 176.5 | 44.5 | 21.35 | 15.7 | 59.5 | 65.33 | 8.05 |

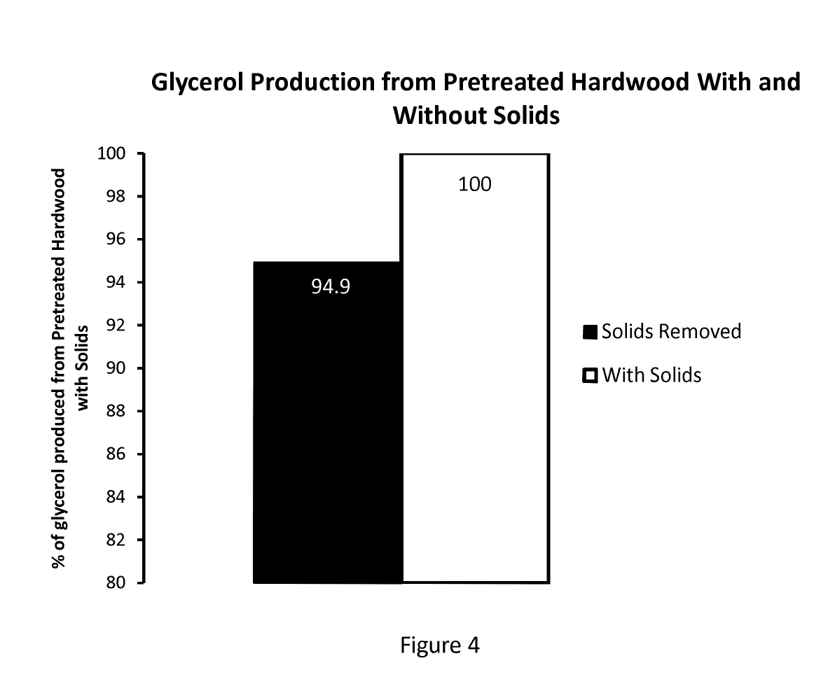

As shown in FIG. 4, the yield of glycerol was about 5.4% higher when the suspended solids were not removed from the sugar stream produced by pretreating and hydrolyzing hardwood as compared to when the suspended solids were removed.

### Example 5

This example examines ethanol and glycerol production during yeast fermentations of saccharide solutions produced from pretreatment and hydrolysis of biomass when exogenous osmotic agents (minerals) or exogenous particulate solids are added to the saccharide solutions.

Methods

Propagation—50 mL of a stock solution containing 10% yeast extract, 1% magnesium sulfate and 0.2% peptone were added to 400 mL of sterile deionized water, along with 50 mL of a sterile 40% dextrose solution in a 1 liter flask. Yeast were added to the propagation solution, which was agitated at 150 RPM at 33° C. for 24 hours.

Saccharide solutions—wheat straw was pretreated and hydrolyzed to produce a saccharide solution containing residual solids (particulate solids). The residual solids containing lignin and other components were removed from the saccharide solution by flocculation and centrifugation to produce a clarified saccharide solution. A portion of the clarified saccharide solution was further subjected to carbon filtration, which removes additional particulates and some soluble components, to produce refined saccharide solutions.

Fermentation—the clarified and refined saccharide solutions were fermented in 35 mL volumes in 50 mL tubes capped with bubblers. Table 6 summarizes the experimental conditions tested. “(+) solids” means that 1.75 g (wet weight) of the particulate solids removed during clarification were added back. “(+) minerals” means that potassium, magnesium, phosphorus, and calcium were added to 1500 ppm, 180 ppm, 170 ppm, and 340 ppm respectively. The pH of each solution was 5.0+/−0.2. The fermentation reactions were performed at 32° C. and with agitation at 110 RPM.

TABLE 6
Experimental design

| Sample 1-4 | Sample 5-8 |
|---|---|
| Clarified Wheat straw | Clarified Wheat straw solution (−) solids |
| solution (−) minerals | |
| Clarified Wheat straw | Clarified Wheat straw solution (+) solids |
| solution (+) minerals | |
| Refined Wheat straw | Refined Wheat straw solution (−) solids |
| solution (−) minerals | |
| Refined Wheat straw | Refined Wheat straw solution (+) solids |
| solution (+) minerals | |

The ethanol production is summarized in Table 7, which shows the percent of sugar converted to ethanol and the conversion efficiency for each sample. The refined wheat straw saccharide solution with 1.75 g wet solids addition per 35 mL had the most sugar converted to ethanol (89.5%) and the highest conversion efficiency of sugar to ethanol (82.2%). The addition of minerals decreased the conversion efficiency and total ethanol produced in both clarified and refined saccharide solutions. The addition of solids did not have a significant effect upon ethanol production.

TABLE 7

| | | | | Sugar | |
|---|---|---|---|---|---|
| | Initial | Residual | Ethanol | converted | Con- |
| | Sugar | Sugar | produced | to | version |
| Sample | (g/L) | (g/L) | (g/L) | ethanol | efficiency |
| Clarified Wheat | 190.7 | 13.3 | 77 | 86.8% | 80.8% |
| straw solution | | | | | |
| (−) minerals | | | | | |
| Clarified Wheat | 190.7 | 12.55 | 71.6 | 80.4% | 75.1% |
| straw solution | | | | | |
| (+) minerals | | | | | |
| Refined Wheat | 186.1 | 15.75 | 74.85 | 87.9% | 80.44 |
| straw solution | | | | | |
| (−) minerals | | | | | |
| Refined Wheat | 186.1 | 15.05 | 69.05 | 80.7% | 74.2% |
| straw solution | | | | | |
| (+) minerals | | | | | |
| Clarified Wheat | 190.7 | 19.05 | 75.5 | 88.2% | 79.2% |
| straw solution | | | | | |
| (−) solids | | | | | |
| Clarified Wheat | 190.7 | 18.9 | 75.85 | 88.3% | 79.5% |
| straw solution | | | | | |
| (+) solids | | | | | |
| Refined Wheat | 186.1 | 14.95 | 74.15 | 86.6% | 79.7% |
| straw solution | | | | | |
| (−) solids | | | | | |
| Refined Wheat | 186.1 | 15.15 | 76.5 | 89.5% | 82.2% |
| straw solution | | | | | |
| (+) solids | | | | | |

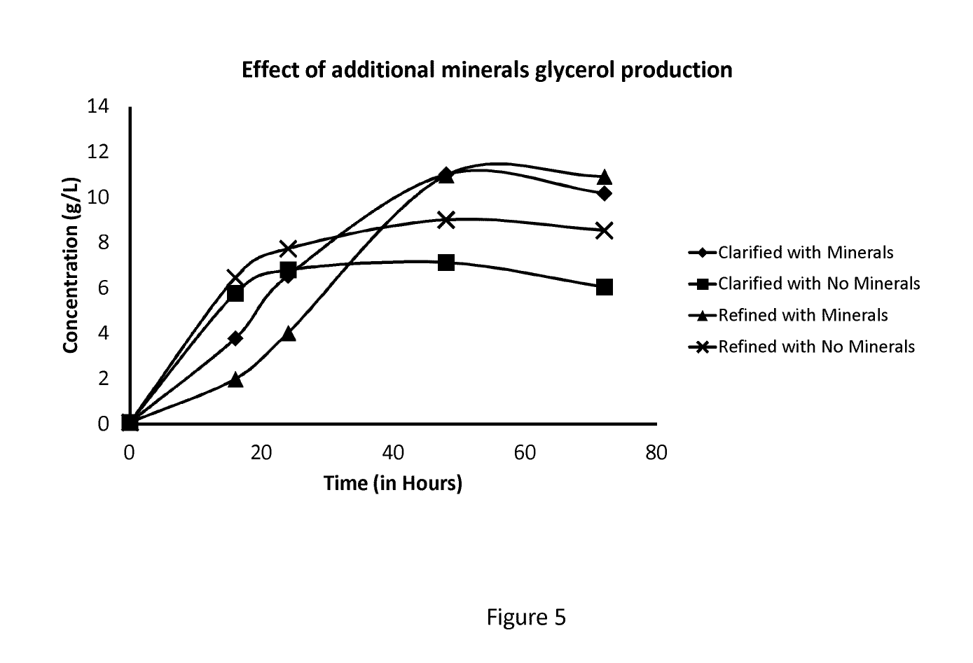

FIG. 5 shows the time-course of glycerol production with and without addition of exogenous minerals. Addition of minerals increased the production of glycerol in both clarified and refined sugar streams. Without additional minerals, the clarified saccharide solution produced a higher level of glycerol than the refined saccharide solution.

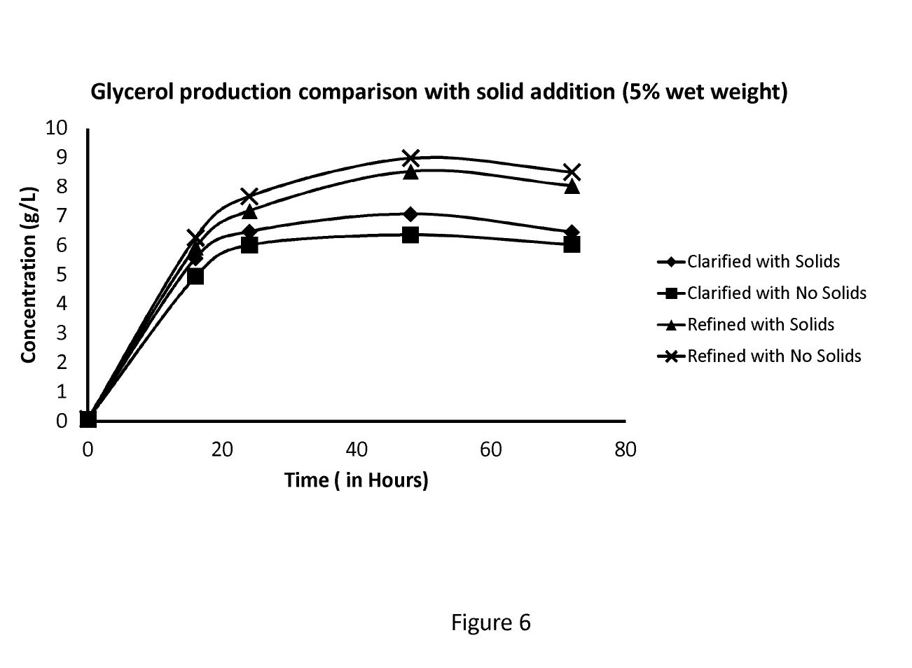

FIG. 6 shows the time-course of glycerol production with and without the addition of particulate solids. Overall, the refined solutions produced more glycerol than the clarified solutions.

### Example 6

The effect of particulate solids (e.g., lignin) on glycerol production during yeast fermentation was examined in this example.

Methods:

Propagation of Oe-2-1 120601 Yeast

Using a sterilized 1 L baffled flask, 50 mL of a stock solution containing 10% yeast extract, 1% MgSO₄ and 0.2% peptone was added to the baffled flask. 50 mL of a stock 40% dextrose solution was added to the flask also. Finally, the total volume of the solution was adjusted up to 500 mL with sterile DI water.

From the freezer, a thawed agar slant that had been struck with the 0e-2-1 120601 yeast (contains about 1 mL of inoculum). The slant was then placed in a 50° C. incubator for 25 minutes. Once the slant was thawed and warm to the touch, 4 mL of the propagation media was added to suspend the inoculum. The inoculum solution was poured back into the propagation flask and placed into the incubator. The incubator was maintained at 33° C. at 150 RPM for 24 hours throughout the course of the propagation.

Preparation of Oat Hulls for Fermentation

Pretreated oat hulls were hydrolyzed for 72 hours using a 50° C. jacketed vessel equipped with an agitating impellar. Cellulase and hemicellulase enzymes from Novozymes were used to break down the oligosaccharides into monomeric form. A sample of those oat hull sugars was sequestered and kept aside after hydrolysis was complete. The particulate solids, including lignin, were sequestered from the remaining at hull sugars by flocculation and centrifugation. Analysis was done on the HPLC to validate the sugar concentration.

Fermentation of Oat Hulls and Pure Sugar Streams with 0e-2-1 120601 Yeast

Based on the HPLC data generated from the flocculated oat hulls, glucose/xylose streams were created using purified sugars to mimic the sugars present within the oat hulls stream. To compare the effects of removing the particulate solids, 2.5 g wet of flocculated oat hull solids were added to one of the pure glucose/xylose streams. This was done by centrifuging the flocculated oat hulls solution and draining off the supernatant. The remaining solids were weighed out and placed into the pure sugar stream flask.

The pH of all solutions was adjusted to 5.0+/−0.1 using sodium hydroxide. 250 uL of a 20% stock urea solution was also added to each flask.

Using an OHAUS MB35 moisture analyzer, the dry weight of yeast (in grams) per 5 mL of propagation solution was determined. Using a proportion based on the number expressed on the moisture analyzer, the amount of solution needed to inoculate four flasks with 0.025 g of dry yeast/25 mL of fermentation broth was calculated. The yeast propagation was spun down, using a centrifuge set at 6000 RPM for 5 minutes. The supernatant was poured off and the yeast was suspended with 1 mL of sterile water. The yeast solution was then sub-divided evenly among the 4 fermentation flasks. Each flask was capped with a bubbler and placed in the incubator at 33° C. Samples were taken throughout the fermentation to observe the rate kinetics.

Results:

Table 8 shows the sugar profile of each of the four sugar solutions that underwent fermentation. These samples were procured and analyzed just prior to yeast inoculation.

TABLE 8
T(0) Samples of Fermentation solutions - Solids added at 5 g wet weight
(0.5% dry weight)

| | | | Arabi- | Glyc- | Acetic | |
|---|---|---|---|---|---|---|
| | Glucose | Xylose | nose | erol | Acid | Ethanol |
| Sample Name | (g/L) | (g/L) | (g/L) | (g/L) | (g/L) | (g/L) |
| Glucose/Xylose | 51.40 | 48.80 | 0.00 | 0.00 | 0.00 | 0.00 |
| with no solids | | | | | | |
| Glucose/Xylose | 50.65 | 50.40 | 0.00 | 0.00 | 0.00 | 0.00 |
| with solids added | | | | | | |
| Oat Hulls - not | 52.40 | 51.90 | 3.80 | 0.00 | 2.75 | 0.00 |
| flocculated | | | | | | |
| Oat Hulls - | 50.00 | 49.45 | 3.65 | 0.00 | 2.65 | 0.00 |
| Flocculated | | | | | | |

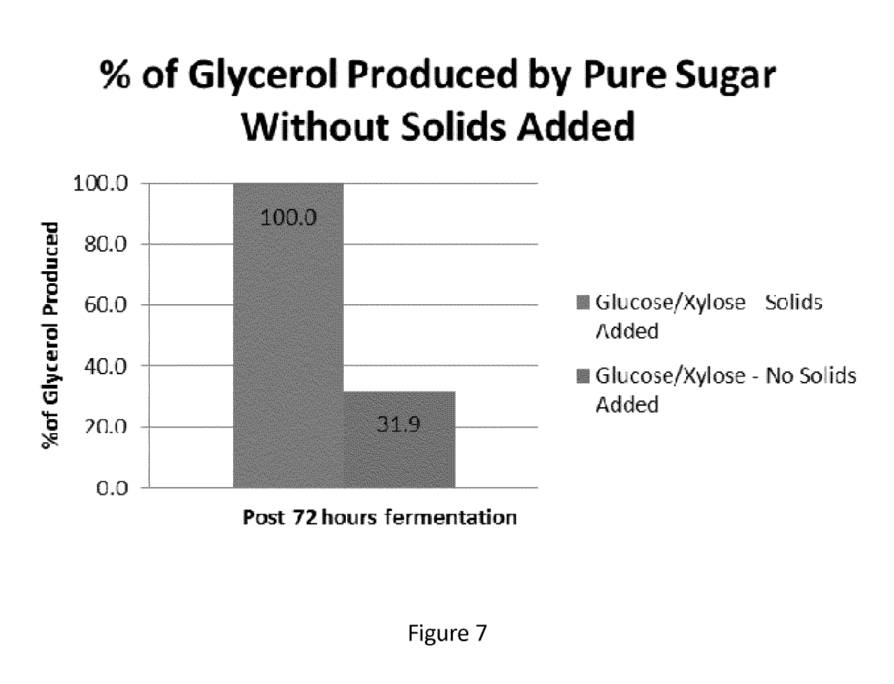

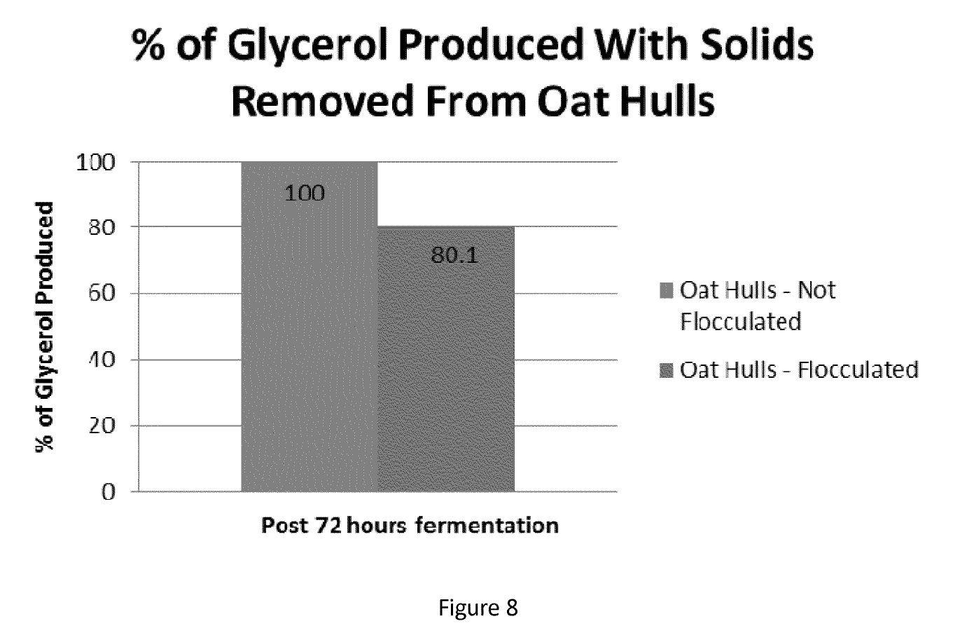

Table 9 shows the initial and final sugar yields and the final ethanol and glycerol levels. The addition of the solids to the pure sugars increased the production of glycerol three fold in comparison to the pure sugars with no solids added. Conversely, the production of ethanol was reduced by a factor of three when the solids were added to the pure sugar stream. Similarly, removing solids from the saccharide stream produced from the pretreatment and hydrolysis of oat hulls decreased glycerol production. Removal of solids from the oat hull saccharides did not significantly impact ethanol production. These results are illustrated in FIG. 7 and FIG. 8.

TABLE 9
Sugar, Ethanol and Glycerol Concentrations

| | Glucose | Glucose | Xylose | Xylose | | |
|---|---|---|---|---|---|---|
| Sample | (T-0) | (T-72) | (T-0) | (T-72) | Ethanol | Glycerol |
| Name | (g/L) | (g/L) | (g/L) | (g/L) | (g/L) | (g/L) |
| Glucose/ | 51.40 | 0.00 | 48.80 | 35.75 | 25.85 | 1.15 |
| Xylose with | | | | | | |
| no solids | | | | | | |
| Glucose/ | 50.65 | 27.35 | 50.40 | 47.67 | 6.75 | 3.60 |
| Xylose with | | | | | | |
| solids added | | | | | | |
| Oat Hulls - | 52.40 | 2.90 | 51.90 | 50.60 | 23.20 | 1.41 |
| not | | | | | | |
| flocculated | | | | | | |
| Oat Hulls - | 50.00 | 2.90 | 49.45 | 42.60 | 23.50 | 1.13 |
| Flocculated | | | | | | |

### Example 7

In this example, three different saccharide streams and two different lignin streams (particulate solids) were subjected to free amino acids profiling (Table 10), trace metals analysis (Table 11), and fatty acid profiling (Table 12). All of the samples were produced from the hydrolysis of pretreated corn stover.

C5+C6 Saccharides Stream & C5+C6 Lignin Stream

Pretreated corn stover containing about 30% solids was used to produce the ‘C5+C6 Saccharide Stream’ and the ‘C5+C6 Lignin Stream’. The solids were placed into a jacketed kettle with an agitator. Water was added to the pretreated solids to create an about 10% solids solution (wt/v). The temperature of the pretreated corn stover was then brought up to about 50° C. The pH was adjusted using ammonium hydroxide to about 5.0. Once pH and temperature are both set, cellulase enzymes were added to the solids at a dosing of about 5% of total dry solids (wt/wt). The solution was kept at about 50° C., a pH of about 5.0 and at constant agitation for about 72 hours. The solids were then separated via filtration from the liquid stream to produce the C5+C6 Lignin Stream at about 20% solids (w/w). The liquid stream at this point contains C5 and C6 monosaccharides. The liquid stream was then concentrated via evaporation to the desired monosaccharide levels to produce the C5+C6 Saccharides Stream. The C5+C6 Saccharides stream contained about 18.7% C6 and about 6.8% C5 saccharides.

C5 Saccharides Stream

Pretreated corn stover containing about 30% solids was used to create the C5 saccharides stream. To extract the monomeric C5 saccharides, hot water (at about 50° C.) was mixed with the pretreated solids at a rate of about 1 L of water for every 1 kg of wet solids. Once the water was mixed with the pretreated solids, the biomass and hot water solution was mixed for about 15 minutes at about 50° C. The solids were then filtered out and the liquid fraction was collected. The liquid fraction was then sequestered. The solids were then re-collected and re-washed with the same ratio of hot water (at about 50° C.) and mixed for about 15 minutes at about 50° C. The solids were then once again filtered out and the liquid fraction was collected and sequestered. The liquid fraction from the second wash was then combined with the liquid fraction from the first wash and the entire liquid fraction was concentrated via evaporation to the desired saccharide levels, yielding the C5 Saccharides Stream. The C5 Saccharides Stream contained about 12.9% C5 saccharides and about 1.3% C6 saccharides.

C6 Saccharides Stream & C6 Lignin Stream

The C6 Saccharides Stream and C6 Lignin Stream are produced from the solids sequestered during production of the C5 Saccharide Stream. The solids were placed into a jacketed kettle with an agitator. Water was added to the pretreated solids to create an about 10% solids solution (w/v). The temperature of the pretreated corn stover was then brought up to about 50° C. The pH was adjusted using ammonium hydroxide to about 5.0. Once pH and temperature were both set, cellulase enzymes (Celtech 3 cellulase from Novozyme) were added to the solids at a dosing of about 5% of total dry solids (wt/wt). The solution was kept at about 50° C., a pH of about 5.0 and at constant agitation for about 72 hours. The solids were then separated from the liquid stream via filtration to produce the C6 Lignin Stream at about 20% solids. The liquid stream at this point is enriched for C6 monosaccharides. The liquid stream was then concentrated via evaporation and vacuum to the desired saccharide levels to produce the C6 Saccharide Stream. The C6 Saccharide Stream contained about 25.1% C6 saccharides and about 2.6% C5 Sugars.

TABLE 10
Amino acids profiling (% w/v)

| | | C6 | C5 | | |
|---|---|---|---|---|---|
| | C5 + C6 | Sac- | Sac- | C5 + C6 | |
| | Saccharides | charides | charides | Lignin | C6 Lignin |
| Amino Acid | Stream | Stream | Stream | Stream | Stream |
| Aspartic | <0.01% | <0.01% | <0.01% | <0.01% | <0.01% |
| Threonine | <0.01% | <0.01% | <0.01% | <0.01% | <0.01% |
| Serine | <0.01% | <0.01% | <0.01% | <0.01% | <0.01% |
| Glutamic Acid | <0.01% | <0.01% | <0.01% | <0.01% | <0.01% |
| Proline | <0.01% | <0.01% | <0.01% | <0.01% | <0.01% |
| Glycine | <0.01% | <0.01% | <0.01% | <0.01% | <0.01% |
| Alanine | <0.01% | <0.01% | <0.01% | <0.01% | <0.01% |
| Cystine, free | <0.01% | <0.01% | <0.01% | <0.01% | <0.01% |
| Valine | <0.01% | <0.01% | <0.01% | <0.01% | <0.01% |
| Methionine | <0.01% | <0.01% | <0.01% | <0.01% | <0.01% |
| Isoleucine | <0.01% | <0.01% | <0.01% | <0.01% | <0.01% |
| Leucine | <0.01% | <0.01% | <0.01% | <0.01% | <0.01% |
| Tyrosine | <0.01% | <0.01% | <0.01% | <0.01% | <0.01% |
| Phenylalanine | <0.01% | <0.01% | <0.01% | <0.01% | <0.01% |
| Lysine | 0.01% | 0.01% | 0.01% | <0.01% | <0.01% |
| Histidine | <0.01% | <0.01% | <0.01% | <0.01% | <0.01% |
| Arginine | <0.01% | <0.01% | <0.01% | <0.01% | 0.05% |

TABLE 11
Trace metal analysis (PPM; Parts Per Million)

| | | | C5 | | |
|---|---|---|---|---|---|
| | C5 + C6 | C6 | Sac- | C5 + C6 | C6 |
| | Saccharides | Saccharides | charides | Lignin | Lignin |
| Metal | Stream | Stream | Stream | Stream | Stream |
| Aluminum | 6.4 | <4.9 | 36 | 25 | 180 |
| Antimony | <0.50 | <0.49 | <0.49 | <0.50 | <0.50 |
| Arsenic | <0.25 | <0.24 | <0.24 | <0.25 | <0.25 |
| Barium | 0.26 | 0.15 | 0.10 | 5.4 | 6.2 |
| Cadmium | <0.099 | <0.097 | <0.097 | <0.10 | <0.099 |
| Calcium | 330 | 86 | 340 | 210 | 1100 |
| Chromium | 1.7 | 0.15 | 5.4 | 6.9 | 15 |
| Cobalt | <0.099 | <0.097 | 0.29 | <0.10 | 0.15 |
| Copper | 1.3 | 0.77 | 4.7 | 6.3 | 10 |
| Iron | 21 | <1.2 | 170 | 89 | 260 |
| Lead | <0.19 | <0.18 | <0.18 | 0.34 | 0.45 |
| Magnesium | 71 | 25 | 180 | 32 | 48 |
| Manganese | 2.0 | 0.26 | 5.1 | 1.0 | 1.2 |
| Nickel | 1.2 | <0.24 | 4.9 | 2.0 | 3.6 |
| Phosphorus | 150 | 95 | 170 | 57 | 34 |
| Potassium | 570 | 97 | 1500 | 200 | 420 |
| Selenium | <0.74 | <0.73 | <0.73 | <0.75 | <0.74 |
| Silver | <0.25 | <0.24 | <0.24 | <0.25 | 0.29 |
| Sodium | 3000 | 480 | 460 | 1100 | 61 |
| Tin | <0.50 | <0.49 | <0.49 | 0.79 | 1.0 |
| Vanadium | <0.30 | <0.49 | <0.49 | <0.50 | 0.51 |
| Zinc | 1.8 | <0.49 | 8.2 | 2.3 | 1.7 |

TABLE 12
Fatty acid profiling (% w/w)

| | C5 + C6 | C6 | C5 | C5 + C6 | C6 |
|---|---|---|---|---|---|
| | Saccharides | Saccharides | Saccharides | Lignin | Lignin |
| Fatty Acid | Stream | Stream | Stream | Stream | Stream |
| C4:0 Butyric | 0.00 | 0.00 | 0.00 | 0.00 | 0.00 |
| C6:0 Hexanoic | 0.00 | 0.00 | 0.00 | 0.00 | 0.00 |
| C8:0 Octanoic | 0.00 | 0.00 | 0.00 | 0.00 | 0.00 |
| C10:0 Decanoic | 0.00 | 0.00 | 0.00 | 0.00 | 0.00 |
| C12:0 Laurie | 0.00 | 0.00 | 0.00 | 0.00 | 0.00 |
| C13:0 Tridecanoic | 0.00 | 0.00 | 0.00 | 0.00 | 0.00 |
| C14:0 Myristic | 0.00 | 0.00 | 0.00 | 0.00 | 0.00 |
| C15:0 Pentadecanoic | 0.00 | 0.00 | 0.00 | 0.00 | 0.00 |
| C16:0 Palmitic | 0.00 | 0.00 | 0.00 | 0.00 | 0.04 |
| C17:0 Heptadecanoic | 0.00 | 0.00 | 0.00 | 0.00 | 0.00 |
| C18:0 Stearic | 0.00 | 0.00 | 0.00 | 0.02 | 0.02 |
| C20:0 Arachidic | 0.00 | 0.00 | 0.00 | 0.00 | 0.01 |
| C21:0 Heneicosanoic | 0.00 | 0.00 | 0.00 | 0.00 | 0.00 |
| C22:0 Behenic | 0.00 | 0.00 | 0.00 | 0.01 | 0.01 |
| C23:0 Tricosanoic | 0.00 | 0.00 | 0.00 | 0.00 | 0.00 |
| C24:0 Lignoceric | 0.00 | 0.00 | 0.00 | 0.01 | 0.01 |
| C14:1 (cis-9) Myristoleic | 0.00 | 0.00 | 0.00 | 0.00 | 0.00 |
| C15:1 (cis-10) Pentadecinoic | 0.00 | 0.00 | 0.00 | 0.00 | 0.04 |
| C16:1 (cis-9) Palmitoleic | 0.00 | 0.00 | 0.00 | 0.00 | 0.00 |
| C17:1 (cis-10) | 0.00 | 0.00 | 0.00 | 0.00 | 0.00 |
| Heptadecenoate | | | | | |
| C18:1 (cis-9) Oleic | 0.00 | 0.00 | 0.00 | 0.05 | 0.06 |
| C20:1 (cis-11) Eicosenoic | 0.00 | 0.00 | 0.00 | 0.00 | 0.00 |
| C22:1 (cis-13) Erucic | 0.00 | 0.00 | 0.00 | 0.00 | 0.00 |
| C24:1 (cis-15) Nervonic | 0.00 | 0.00 | 0.00 | 0.00 | 0.00 |
| C18:2 (cis-9,12) Lonoleic | 0.00 | 0.00 | 0.00 | 0.07 | 0.05 |
| C18:3 (cis-6,9,12) y- | 0.00 | 0.00 | 0.00 | 0.00 | 0.00 |
| linolenic | | | | | |
| C18:3 (cis-9,12,15) | 0.00 | 0.00 | 0.00 | 0.02 | 0.00 |
| Linolenic | | | | | |
| C20:2 (cis-11,14) | 0.00 | 0.00 | 0.00 | 0.00 | 0.00 |
| Eicosadienoic | | | | | |
| C20:3 (cis-8,11,14) | 0.00 | 0.00 | 0.00 | 0.00 | 0.00 |
| Eicosatrienoic | | | | | |
| C20:3 (cis-11,14,17) | 0.00 | 0.00 | 0.00 | 0.00 | 0.00 |
| Eicosatrienoic | | | | | |
| C20:4 (cis-5,8,11,14) | 0.00 | 0.00 | 0.00 | 0.00 | 0.00 |
| Arachidonic | | | | | |
| C20:5 (cis-5,8,11,14,17) | 0.00 | 0.00 | 0.00 | 0.00 | 0.00 |
| Eicosapentanoic | | | | | |
| C22:2 (cis-13,16) | 0.00 | 0.00 | 0.00 | 0.00 | 0.01 |
| Docosadienoic | | | | | |
| C22:6 (cis-4,7,10,13,16, | 0.00 | 0.00 | 0.00 | 0.00 | 0.00 |
| 19) | | | | | |
| Docosahexaenoic | | | | | |
| C18:1 (trans-9) Methyl | 0.00 | 0.00 | 0.00 | 0.01 | 0.00 |
| Elaidate | | | | | |
| C18:2 (trans-9,12) Methyl | 0.00 | 0.00 | 0.00 | 0.00 | 0.00 |
| Linoelaidate | | | | | |

### Example 8

In this example, the production of castor oil by plant cells (Ricinus communis) cultured with saccharide streams containing higher amounts of particulate solids and/or osmotic agents is increased. In some conditions, exogenous osmotic agents such as salts or minerals are added to the saccharide streams to increase the osmolarity of the solution. In some conditions, exogenous particulate solids are added to the saccharide streams to increase the solids content of the solution. The exogenous particulate solids can contain residual solids from the pretreatment and hydrolysis of biomass and can contain lignin, cellulose, and/or hemicellulose particles. In some conditions, the saccharide stream contains osmotic agents (e.g., salts, acid solubilized lignin, fatty acids, metal ions trace elements, acids, bases, ash, organic acids, alcohols, etc.) from the pretreatment and hydrolysis of biomass. In some conditions, the saccharide streams contain particulate solids that are residual solids from the pretreatment and/or hydrolysis of biomass. The castor oil produced by the plant cells contains substantially the same composition of fatty acids as commercially available castor oil.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure herein. It should be understood that various alternatives to the embodiments of the disclosure described herein can be employed in practicing the described subject matter. It is intended that the following claims define the scope of the disclosure and that methods and structures within the scope of these claims and their equivalents be covered thereby.

1.-320. (canceled)

321. A method of producing one or more fermentation end-products comprising:
(a) contacting a saccharide solution comprising C5 and/or C6 monosaccharides, and particulate solids, with a cell culture; and
(b) allowing sufficient time for cells in the cell culture to produce one or more fermentation end-products comprising one or more polyols, fatty acids, triacylglycerols, or a combination thereof; wherein a greater yield of the one or more polyols, fatty acids, or triacylglycerols is produced in comparison to fermentation of a saccharide solution comprising a lower level of the particulate solids or one or more osmotic agents.

322. The method of claim 321, wherein the particulate solids comprise lignin, cellulose, hemicellulose, or a combination thereof.

323. The method of claim 321, wherein the particulate solids are residual solids from pretreating and hydrolyzing a biomass.

324. The method of claim 321, wherein the saccharides solution further comprises one or more osmotic agents.

325. The method of claim 324, wherein the one or more osmotic agents comprise one or more salts, acid solubilized lignin, one or more fatty acids, one or more metal ions, one or more trace elements, one or more acids, one or more bases, ash, one or more organic acids, one or more alcohols, or a combination thereof.

326. The method of claim 321, further comprising adding exogenous particulate solids or one or more exogenous osmotic agents to the saccharide solution.

327. The method of claim 321, wherein the cell culture comprises plant cells, bacterial cells, yeast cells, algal cells, or a combination thereof.

328. The method of claim 321, wherein the one or more fermentation end-products comprises glycerol.

329. The method of claim 321, wherein the saccharide solution was produced by pretreating and hydrolyzing a biomass composition comprising cellulosic, hemicellulosic, and/or lignocellulosic material.

330. A method of producing one or more fermentation end-products comprising:
(a) contacting a saccharide solution comprising C5 and/or C6 monosaccharides, and one or more osmotic agents, with a cell culture; and
(b) allowing sufficient time for cells in the cell culture to produce one or more fermentation end-products comprising one or more polyols, fatty acids, triacylglycerols, or a combination thereof; wherein a greater yield of the one or more polyols, fatty acids, or triacylglycerols is produced in comparison to fermentation of a saccharide solution comprising a lower level of the one or more osmotic agents.

331. The method of claim 330, wherein the one or more osmotic agents comprise one or more salts, acid solubilized lignin, one or more fatty acids, one or more metal ions, one or more trace elements, one or more acids, one or more bases, ash, one or more organic acids, one or more alcohols, or a combination thereof.

332. A system for producing an increased yield of polyols, fatty acids, and/or triacylglycerols, the system comprising:
(a) a fermentation vessel;
(b) a saccharide solution comprising C5 and/or C6 monosaccharides, and particulate solids or one or more osmotic agents; and
(c) a cell culture comprising cells that produce one or more polyols, fatty acids and/or triacylglycerols from the C5 and/or C6 monosaccharides in a greater yield than from an equivalent amount of the C5 and/or C6 monosaccharides with a lower level of the particulate solids or the one or more osmotic agents.

333. The system of claim 332, wherein the saccharide solution comprises the particulate solids.

334. The system of claim 333, wherein the particulate solids comprise lignin, cellulose, hemicellulose, or a combination thereof.

335. The system of claim 333, wherein the particulate solids are residual solids from pretreating and hydrolyzing a biomass.

336. The system of claim 332, wherein the saccharide solution comprises the one or more osmotic agents.

337. The system of claim 336, wherein the one or more osmotic agents comprise one or more salts, acid solubilized lignin, one or more fatty acids, one or more metal ions, one or more trace elements, one or more acids, one or more bases, ash, one or more organic acids, one or more alcohols, or a combination thereof.

338. The system of claim 332, wherein the cell culture comprises plant cells, bacterial cells, yeast cells, algal cells, or a combination thereof.

339. The system of claim 332, wherein the one or more fermentation end-products comprises glycerol.

340. The system of claim 332, wherein the saccharide solution was produced by pretreating and hydrolyzing a biomass composition comprising cellulosic, hemicellulosic, and/or lignocellulosic material.
